SEQUENCE LISTING (37 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 153 aa, Homo sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 3 - DNA, 716 bp, Homo sapiens
agcaggctcagaaggtccagaaatcaggggaaggagacccctatctgtccttcttctggaagagctggaaaggaagtctgctcaggaaataaccttggaagatggtggccacgaagacctttgctctgctgctgctgtccctgttcctggcagtgggactaggagagaagaaagagggtcacttcagcgctctcccctccctgcctgttggatctcatgctaaggtgagcagccctcaacctcgaggccccaggtacgcggaagggactttcatcagtgactacagtattgccatggacaagattcaccaacaagactttgtgaactggctgctggcccaaaaggggaagaagaatgactggaaacacaacatcacccagagggaggctcgggcgctggagctggccagtcaagctaataggaaggaggaggaggcagtggagccacagagctccccagccaagaaccccagcgatgaagatttgctgcgggacttgctgattcaagagctgttggcctgcttgctggatcagacaaacctctgcaggctcaggtctcggtgactctgaccacacccagctcaggactggattctgcccttcacttagcacctgcctcagccccactccagaatagccaagagaacccaaaccaataaagtttatgctaagtcgagcccattgtgaaaatttattaaaatgactactgagcactaa

SEQ ID NO: 4 - protein, 42 aa, Artificial Sequence
  Compound 1
YAEGTFISDYSIAMDKIHQQDFVNWLLAQKGKKNDWKHNIIQ

SEQ ID NO: 5 - protein, 42 aa, Artificial Sequence; identical to SEQ ID NO: 4
  Compound 2
  MISC_FEATURE (2)..(2): D-Alanine
  MISC_FEATURE (37)..(37): Lys conjugated to palmitoyl acyl fatty acid

SEQ ID NO: 6 - protein, 42 aa, Artificial Sequence; identical to SEQ ID NO: 4
  Compound 3
  MISC_FEATURE (16)..(16): Lys conjugated to palmitoyl acyl fatty acid

SEQ ID NO: 7 - protein, 30 aa, Artificial Sequence
  Compound 4
  MISC_FEATURE (2)..(2): D-Alanine
YAEGTFISDYSIAMDKIHQQDFVNWLLAQK

SEQ ID NO: 8 - protein, 42 aa, Artificial Sequence
  Compound 5
  MISC_FEATURE (2)..(2): 2-Aminoisobutyric acid (Aib)
  MISC_FEATURE (17)..(17): Lys conjugated to g-Glu-C16
YXEGTFISDYSIAMDKKHQQDFVNWLLAQKGKKNDWKHNIIQ

SEQ ID NO: 9 - protein, 29 aa, Artificial Sequence
  Compound 6
  MISC_FEATURE (2)..(2): 2-Aminoisobutyric acid (Aib)
  MISC_FEATURE (20)..(20): Lys conjugated to palmitoyl acyl fatty acid
YXEGTFTSDYSIYMEKEAVKEFIAWLVKG

SEQ ID NO: 10 - protein, 30 aa, Artificial Sequence
  Compound 7
  MISC_FEATURE (1)..(1): D-Tyrosine
  MISC_FEATURE (30)..(30): Lys conjugated to palmitoyl acyl fatty acid
YAEGTFISDYSIAMDAIHQQDFVNWLLAQK

SEQ ID NO: 11 - protein, 42 aa, Artificial Sequence
  Compound 8
  MISC_FEATURE (1)..(1): 3,5-diBr-Tyrosine
  MISC_FEATURE (2)..(2): 2-aminoisobutyric acid (Aib)
  MISC_FEATURE (13)..(13): 2-aminoisobutyric acid (Aib)
  MISC_FEATURE (14)..(14): Norleucine (Nle)
YXEGTFISDYSIXXDKIHQQDFVNWLLAQKGKKNDWKHNITQ

SEQ ID NO: 12 - protein, 42 aa, Artificial Sequence
  Compound 9
  MISC_FEATURE (2)..(2): 2-aminoisobutyric acid  (Aib)
  MISC_FEATURE (11)..(11): 1-NH,1-cyclopentane carboxylic acid
  MISC_FEATURE (14)..(14): Norleucine (Nle)
FXEGTFISDYXIAXDKIHQQDFVNWLLAQKGKKNDWKHNITQ

SEQ ID NO: 13 - protein, 40 aa, Artificial Sequence
  Compound 10
  MISC_FEATURE (2)..(2): 2-Aminoisobutyric acid (Aib)
  MISC_FEATURE (20)..(20): 2-Aminoisobutyric acid (Aib)
  MISC_FEATURE (40)..(40): Lys conjugated to palmitoyl acyl fatty acid
YXEGTFTSDYSIYLDKQAAXEFVNWLLAGGPSSGAPPPSK

SEQ ID NO: 14 - protein, 39 aa, Artificial Sequence
  Compound 11
  MISC_FEATURE (2)..(2): D-Alanine
YAEGTFISDYSIAMDKIHQQDFVNWLLAQKPSSGAPPPS

SEQ ID NO: 15 - protein, 39 aa, Artificial Sequence
  Compound 12
YAPGTFISDYSIAMDKIHQQDFVNWLLAQKPSSGAPPPS

SEQ ID NO: 16 - protein, 42 aa, Artificial Sequence; identical to SEQ ID NO: 4
  Compound 13
  MISC_FEATURE (2)..(2): D-Alanine
  MISC_FEATURE (16)..(16): Lys conjugated to (OEG-gGlu)x2-Oda)

SEQ ID NO: 17 - protein, 42 aa, Artificial Sequence; identical to SEQ ID NO: 4
  Compound 14
  MISC_FEATURE (2)..(2): D-Alanine

SEQ ID NO: 18 - protein, 40 aa, Artificial Sequence
  Compound 15
  MISC_FEATURE (2)..(2): 2-Aminoisobutyric acid (Aib)
  MISC_FEATURE (40)..(40): Lys conjugated to an acyl group
YXEGTFTSDYSIYMEKEAVREFIAWLVKGGPSSGAPPPSK

SEQ ID NO: 19 - protein, 43 aa, Artificial Sequence
  Compound 16
  MISC_FEATURE (2)..(2): 2-Aminoisobutyric acid (Aib)
  MISC_FEATURE (13)..(13): 2-Aminoisobutyric acid (Aib)
  MISC_FEATURE (29)..(29): 2-Aminoisobutyric acid (Aib)
  MISC_FEATURE (43)..(43): Lys conjugated to acyl group
YXEGTFTSDYSIXLDKIAQRAFVQWLIAXKGKKQEWKHQITQK

SEQ ID NO: 20 - protein, 40 aa, Artificial Sequence; identical to SEQ ID NO: 13
  Compound 17
  MISC_FEATURE (2)..(2): 2-Aminoisobutyric acid (Aib)
  MISC_FEATURE (20)..(20): 2-Aminoisobutyric acid (Aib)
  MISC_FEATURE (40)..(40): Lys conjugated to acyl group

SEQ ID NO: 21 - protein, 39 aa, Artificial Sequence
  Compound 18
  MISC_FEATURE (2)..(2): 2-Aminoisobutyric acid (Aib)
  MISC_FEATURE (13)..(13): 2-Aminoisobutyric acid (Aib)
  MISC_FEATURE (20)..(20): Lys conjugated to (ethylene glycol) x2-C20 diacid
YXEGTFTSDYSIXLDKIAQKAFVQWLIAGGPSSGAPPPS

SEQ ID NO: 22 - protein, 43 aa, Artificial Sequence
  Compound 19
  MISC_FEATURE (2)..(2): 2-Aminoisobutyric acid (Aib)
  MISC_FEATURE (43)..(43): Lys conjugated to (gamma-glutamic acid) x3-palmitoyl
YXEGTFISDYSIAMDKIHQQDFVNWLLAQKGKKNDWKHNITQK

SEQ ID NO: 23 - protein, 39 aa, Artificial Sequence
  Compound 20
  MISC_FEATURE (2)..(2): 2-Aminoisobutyric acid (Aib)
  MISC_FEATURE (17)..(17): Lys conjugated to (ethylene glycol)x2-C15 diacid
YXEGTFISDYSIELDKKAAQAFIEWLLAQGPSSGAPPPS

SEQ ID NO: 24 - protein, 39 aa, Artificial Sequence
  Compound 21
  MISC_FEATURE (2)..(2): 2-Aminoisobutyric acid (Aib)
  MISC_FEATURE (39)..(39): Lys conjugated to (ethylene glycol)x2-C15 diacid
YXEGTFISDYSIELDKIAAQDFIEWLLAGPSSGAPPPSK

SEQ ID NO: 25 - protein, 39 aa, Artificial Sequence
  Compound 22
  MISC_FEATURE (2)..(2): 2-Aminoisobutyric acid (Aib)
  MISC_FEATURE (12)..(12): Lys conjugated to (ethylene glycol)x2-C15 diacid
YXEGTFISDYSKELDKIAQRAFIEWLLAQGPSSGAPPPS

SEQ ID NO: 26 - protein, 39 aa, Artificial Sequence
  Compound 23
  MISC_FEATURE (2)..(2): 2-Aminoisobutyric acid
  MISC_FEATURE (17)..(17): Lys conjugated to (ethylene glycol)x2-C15 diacid
YXEGTFISDYSIELEKKAQRAFVEWLLAQGPSSGAPPPS

SEQ ID NO: 27 - protein, 31 aa, Artificial Sequence
  Compound 24
  MISC_FEATURE (2)..(2): 2-Aminoisobutyric acid
  MISC_FEATURE (21)..(21): Lys conjugated to (ethylene glycol)x2-C15 diacid
YXEGTFISDYSIALDKIHQQKFVNWLLAQRG

SEQ ID NO: 28 - protein, 31 aa, Artificial Sequence
  Compound 25
  MISC_FEATURE (2)..(2): 2-Aminoisobutyric acid
  MISC_FEATURE (18)..(18): Lys conjugated to (ethylene glycol)x2-C15 diacid
YXEGTFISDYSIALDKIKQQDFVNWLLAQRG

SEQ ID NO: 29 - protein, 31 aa, Artificial Sequence
  Compound 26
  MISC_FEATURE (2)..(2): 2-Aminoisobutyric acid (Aib)
  MISC_FEATURE (17)..(17): Lys conjugated to (ethylene glycol)x2-C15 diacid
YXEGTFISDYSIALDKKHQQDFVNWLLAQRG

SEQ ID NO: 30 - protein, 30 aa, Artificial Sequence
  Compound 27
  MISC_FEATURE (2)..(2): 2-Aminoisobutyric acid
  MISC_FEATURE (17)..(17): Lys conjugated to (ethylene glycol)x2-C15 diacid
YXEGTFISDYSIALDKKHQQDFVNWLLAQR

SEQ ID NO: 31 - protein, 30 aa, Artificial Sequence
  Compound 28
  MISC_FEATURE (2)..(2): 2-Aminoisobutyric acid (Aib)
  MISC_FEATURE (21)..(21): Lys conjugated to (ethylene glycol)x2-C15 diacid
YXEGTFISDYSIALDKIHQQKFVNWLLAQR

SEQ ID NO: 32 - protein, 39 aa, Artificial Sequence
  Compound 29
  MISC_FEATURE (2)..(2): 2-Aminoisobutyric acid (Aib)
  MISC_FEATURE (21)..(21): Lys conjugated to (ethylene glycol)x2-C15 diacid
YXEGTFISDYSIALDKIHQQKFVNWLLAQRPSSGAPPPS

SEQ ID NO: 33 - protein, 39 aa, Artificial Sequence
  Compound 30
  MISC_FEATURE (2)..(2): 2-Aminoisobutyric acid (Aib)
  MISC_FEATURE (18)..(18): Lys conjugated to (ethylene glycol)x2-C15 diacid
YXEGTFISDYSIALDKIKQQDFVNWLLAQRPSSGAPPPS

SEQ ID NO: 34 - protein, 31 aa, Artificial Sequence
  Compound 31
  MISC_FEATURE (2)..(2): 2-Aminoisobutyric acid (Aib)
  MISC_FEATURE (30)..(30): Lys conjugated to (ethylene glycol)x2-C15 diacid
YXEGTFISDYSIALDKIHQQDFVNWLLAQKG

SEQ ID NO: 35 - protein, 31 aa, Artificial Sequence
  Compound 32
  MISC_FEATURE (2)..(2): 2-Aminoisobutyric acid (Aib)
  MISC_FEATURE (12)..(12): Lys conjugated to (ethylene glycol)x2-C15 diacid
YXEGTFISDYSKALDKIHQQDFVNWLLAQRG

SEQ ID NO: 36 - protein, 39 aa, Artificial Sequence; identical to SEQ ID NO: 21
  Compound 33
  MISC_FEATURE (2)..(2): 2-Aminoisobutyric acid (Aib)
  MISC_FEATURE (13)..(13): 2-Aminoisobutyric acid (Aib)
  MISC_FEATURE (20)..(20): Lys conjugated to (ethylene glycol)x2-C15 diacid

SEQ ID NO: 37 - protein, 39 aa, Artificial Sequence; identical to SEQ ID NO: 21
  Compound 34
  MISC_FEATURE (2)..(2): 2-Aminoisobutyric acid (Aib)
  MISC_FEATURE (13)..(13): 2-Aminoisobutyric acid (Aib)
  MISC_FEATURE (20)..(20): Lys conjugated to (ethylene glycol)x2-C20 diacid

(19) United States
(12) Patent Application Publication
(10) Pub. No.: US 2022/0016215 A1
(43) Pub. Date: Jan. 20, 2022

(54) GIP RECEPTOR AGONIST PEPTIDE COMPOUNDS AND USES THEREOF

(71) Applicant: TAKEDA PHAARMACEUTICAL COMPANY LIMITED, Osaka-shi, Osaka (JP)

(72) Inventors: Shiro TAKEKAWA, Fujisawa-shi (JP); Tomoko MORIMOTO, Fujisawa-shi (JP); Minoru MARUYAMA, Fujisawa-shi (JP); Hiroaki NAGAI, Fujisawa-shi (JP); Akira TANAKA, Fujisawa-shi (JP); Hisanori MATSUI, Fujisawa-shi (JP); Atsuko OBINATA, Fujisawa-shi (JP); Derek Cecil COLE, San Diego, CA (US); Mack FLINSPACH, San Diego, CA (US); Nick SCORAH, San Diego, CA (US); Abhijit Suresh BHAT, Cambridge, MA (US)

(21) Appl. No.: 17/279,228

(22) PCT Filed: Sep. 24, 2019
(86) PCT No.: PCT/JP2019/038827

§ 371 Date: Mar. 24, 2021

Related U.S. Application Data
(60) Provisional application No. 62/735,510, filed on Sep. 24, 2018.

(51) Int. Cl. A61K 38/22; A61P 1/08

(52) U.S. Cl. CPC A61K 38/22; A61P 1/08

(57) ABSTRACT

The present disclosure provides GIF receptor agonist peptide compounds having an activating action on GIF receptors and use of the GIF receptor agonist peptide as a medicament for the treatment and/or prevention of emesis. Specifically, a GIF receptor agonist peptide containing a sequence represented by the formula (I) or a salt thereof, and a medicament comprising the same are N provided. Formula (I) P¹-A1-A2-A3-A4-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-P², wherein each symbol is as defined herein, with the proviso that the GIF receptor agonist peptide is not native human GIF having an amino acid sequence as set forth in SEQ ID NO: 1.

### CROSS-REFERENCE TO RELATED APPLICATION

This application claims the benefit of priority from U.S. Provisional Application Ser. No. 62/735,510 filed on Sep. 24, 2018, the entire content of which is incorporated herein by reference in its entirety.

### TECHNICAL FIELD

The present disclosure relates to peptide compounds having an activating action on GIP receptors and use of the peptide compound as a medicament.

### BACKGROUND

The statements in this section merely provide background information related to the present disclosure and may not constitute prior art.

Both glucagon-like peptide-1 (GLP-1) and glucose-dependent insulinotropic polypeptide (GIP) are peptides called incretins. GLP-1 and GIP are secreted from small intestinal L cells and K cells, respectively.

GLP-1 acts via GLP-1 receptors and is known to have a glucose-dependent insulinotropic action and a feeding suppressive action. On the other hand, GIP is known to have a glucose-dependent insulinotropic action via GIP receptors, though the influence of GIP on feeding is not clear.

Attempts have been made to search for peptides having GLP-1 receptor/GIP receptor coagonist or glucagon receptor/GLP-1 receptor/GIP receptor triagonist activity and modifications thereof and develop these peptides as anti-obesity drugs, therapeutic drugs for diabetes, or therapeutic drugs for neurodegenerative disorders on the basis of the structure of natural glucagon, GIP, or GLP-1. However, the peptide compound and the compound having an activating action on GIP receptors of the present disclosure for the use in treating emesis and similar symptoms associated with emesis such as nausea and vomiting are not disclosed.

### SUMMARY

It is an object of the present disclosure to provide a novel GIP receptor agonist peptide which has a GIP receptor activation action and is useful as an preventive/therapeutic antiemetic agent, for the prevention and treatment of conditions, diseases, and disorders accompanied by vomiting and/or nausea.

The present inventors have carried out extensive studies to solve the above problem and found GIP receptor agonist peptide compounds comprising the sequence represented by formulas (I), (II), (III), (IV), (V), (VI), and (VII) as compounds having a GIP receptor activation action. Further, the present disclosure provides GIP receptor agonist compounds that selectively activate the GIP receptor and have an antiemetic action and be used to treat and/or prevent emesis.

The present disclosure includes the following embodiments (1) to (66).

(1). In some embodiments, the present disclosure provides a method for treating emesis in a subject, for example, in a subject in need thereof. The method comprises administering a therapeutically effective amount of a GIP receptor agonist peptide, or a salt thereof, with the proviso that the GIP receptor agonist peptide does not have an amino acid sequence as set forth in SEQ ID NO: 1.

(2). In some embodiments, the present disclosure provides a method for treating emesis in a subject, for example, in a subject in need thereof. The method comprises administering a therapeutically effective amount of a GIP receptor agonist peptide, or a salt thereof, with the proviso that the peptide does not have an amino acid sequence as set forth in any one of SEQ ID NOs: 4 to 569 disclosed in PCT Application No. PCT/JP2018/013540.

(3). In some embodiments, the present disclosure provides a method for treating emesis in a subject, for example, in a subject in need thereof. The method comprises administering a therapeutically effective amount of a GIP receptor agonist peptide, or a salt thereof, wherein the GIP receptor agonist peptide has at least 80% sequence identity to amino acid sequence of the native human GIP peptide having an amino acid sequence as set forth in SEQ ID NO: 1, with the proviso that the peptide does not have an amino sequence as set forth in SEQ ID NO: 1.

(4). In some embodiments, the present disclosure provides a method for treating emesis in a subject, for example, in a subject in need thereof. The method comprises administering a therapeutically effective amount of a GIP receptor agonist peptide having 29 or 30 amino acids, or a salt thereof.

(5). In some embodiments, the present disclosure provides a method for treating emesis in a subject, for example, in a subject in need thereof. The method comprises administering a therapeutically effective amount of a GIP receptor agonist peptide, or a salt thereof, wherein the GIP receptor agonist peptide has at least 80%, at least 85%, at least 90%, at least 95%, at least 96%, or at least 97%, sequence identity to amino acid sequence of the native human GIP peptide having an amino acid sequence as set forth in SEQ ID NO: 1, with the proviso that the peptide does not have an amino sequence as set forth in SEQ ID NO: 1.

(6). In some embodiments, the present disclosure provides a method for treating emesis in a subject, for example, in a subject in need thereof. The method comprises administering a therapeutically effective amount of a GIP receptor agonist peptide having 29 or 30 amino acids, or a salt thereof, wherein the GIP receptor agonist peptide has at least 80% sequence identity to amino acid sequence 1-29 or 1-30 of the native human GIP peptide having an amino acid sequence as set forth in SEQ ID NO: 1.

(7). In some embodiments, the present disclosure provides a method for treating emesis in a subject, for example, in a subject in need thereof. The method comprises administering a therapeutically effective amount of a GIP receptor agonist peptide having 29 or 30 amino acids, or a salt thereof, wherein the GIP receptor agonist peptide has at least 80% sequence identity to amino acid sequence 1-29 or 1-30 of the native human GIP peptide having an amino acid sequence as set forth in SEQ ID NO: 1, with the proviso that the GIP receptor agonist peptide does not have an amino acid sequence as set forth in any 29 or 30 amino acid peptide provided in SEQ ID NOs: 4 to 569 disclosed in PCT Application No. PCT/JP2018/013540.

(8). In some embodiments, the present disclosure provides a method for treating emesis in a subject, for example, in a subject in need thereof. The method comprises administering a therapeutically effective amount of a GIP receptor agonist peptide, or a salt thereof, said peptide having the formula (I): P¹-A1-A2-A3-A4-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or

is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, or des-amino-Phe, or des-amino-Tyr;
A2 represents Ala, Aib, D-Ala, Gly, Ser, or Sar;
A3 represents Glu or Pro;
A4 represents Gly, or Ser;
A5 represents Thr, D-Iva, Glu, Iva, or Ser;
A6 represents Phe, Iva, Val, Ala, Aib, Cha, or α-methyl-Leu;
A7 represents Ile, Lys, Ala, Aib, Cha, D-Leu, Ile, Thr, Arg, or Val;
A8 represents Ser, Ala, ψ, or Aib;
A9 represents Asp, Leu, ψ, Phe, Glu, or Gln;
A10 represents Tyr, Leu, Ser, Cha, or ψ;
A11 represents Ser, Aib, A5c, A6c, D-Iva, or Iva;
A12 represents Ile, Lys, Glu, Asp, Ala, Aib, Lys-Ac, Ser, α-methyl-Phe, or ψ;
A13 represents Ala, Aib, Tyr, D-Iva, ψ, Gln, Leu, Glu, or Iva;
A14 represents Met, Nle, α-methyl-Leu, Leu, or ψ;
A15 represents Asp, Glu, Lys, Ser, Tyr, ψ, or Asn;
A16 represents Lys, Ala, Ser, Glu, Arg, Aib, Lys-Ac, or ψ;
A17 represents Ile, Lys, Arg, Aib, Gln, Glu, Lys-Ac, or ψ;
A18 represents His, Arg, Ala, Aib, D-Iva, Phe, Iva, Leu, Ser, Trp, or ψ;
A19 represents Gln, Lys, Glu, Ala, Val, Ser, Aib, Arg, or ψ;
A20 represents Gln, Lys, Ala, His, Arg, Aib, Asp, Gly, or ψ;
A21 represents Asp, Leu, Asn, Asp, Glu, Ala, Leu, Ser, Aib, or ψ;
A22 represents Phe, α-methyl-Phe, Naphthyl-Ala, Asn, Ala, Trp, or ψ;
A23 represents Val, Ile, or ψ;
A24 represents Asn, Asp, Glu, Ala, Aib, Gln, Glu, Lys, Lys-Ac, Leu, Nle, Arg, Ser, or ψ;
A25 represents Trp, Tyr, Glu, Phe, Arg, α-methyl-Phe, or ψ;
A26 represents Leu, Aib, Iva, Leu, Nle, or ψ;
A27 represents Leu, Glu, Ser, Lys, Val, Ile, Nle, or ψ;
A28 represents Ala, Ser, Arg, Leu, Met, Lys, Lys-Ac, Aib, or ψ;
A29 represents Gln, Gly, Ala, Thr, Ser, Lys, Aib, ψ, or deletion;
A30 represents Lys, Arg, Gly, Pro, Glu, Lys-Ac, ψ, or deletion;
A31 represents Phe, Pro, Gly, ψ, or deletion;
A32 represents Lys, Ser, Gly, ψ, or deletion;
A33 represents Lys, Ser, Gly, Ile, Ser, ψ, or deletion;
A34 represents Asn, Ala, Gly, Gln, ψ, or deletion;
A35 represents Asp, Ala, Ser, Pro, Glu, ψ, or deletion;
A36 represents Trp, Pro, Gly, ψ, or deletion;
A37 represents Lys, Pro, Gly, ψ, or deletion;
A38 represents His, Pro, Gly, Ser, ψ, or deletion;
A39 represents Asn, Ser, Gly, Asn, Lys, Gln, ψ, or deletion;
A40 represents Ile, Arg, Glu, Lys, Ser, Lys-Ac, Arg, ψ, or deletion;
A41 represents Ile, Thr, Gly, ψ, or deletion;
A42 represents Gln, Gly, ψ, or deletion;
A43 represents ψ, or deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

(9). In some embodiments, the present disclosure provides a method for treating emesis in a subject, for example, in a subject in need thereof. The method comprises administering a therapeutically effective amount of a GIP receptor agonist peptide, or a salt thereof, said peptide having the formula (II): P¹-A1-A2-A3-Gly-Thr-A6-A7-Ser-A9-A10-A11-A12-A13-A14-A15-A16-A17-A18-A19-A20-A21-A22-A23-A24-Trp-Leu-A27-A28-A29-A30-A31-A32-A33-A34-A35-A36-A37-A38-A39-A40-A41-A42-A43-P²,

wherein
P¹ represents a group represented by formula

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent;

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, des-amino-Phe, or des-amino-Tyr;
A2 represents Ala, Aib, D-Ala, Gly, Ser, or Sar;
A3 represents Glu, or Pro;
A6 represents Phe, Iva, or Val;
A7 represents Ile, Lys, Thr, or Val;
A9 represents Asp, Leu, or Phe;
A10 represents Tyr, or ψ;
A11 represents Ser, A5c, Leu, Aib, or Cha;
A12 represents Ile, Lys, Glu, Asp, or ψ;
A13 represents Ala, Aib, Tyr, D-Iva, Gln, Leu, or Glu;
A14 represents Met, Nle, Leu, or ψ;
A15 represents Asp, Glu, Lys, Ser, or Tyr;
A16 represents Lys, Ala, Ser, Glu, Arg, or ψ;
A17 represents Ile, Lys, Arg, Aib, Gln, Ile, Glu, or ψ;
A18 represents His, Ala, or ψ;
A19 represents Gln, Lys, Glu, Ala, Val, Ser, or ψ;
A20 represents Gln, Lys, Ala, His, Arg, Aib, or ψ;
A21 represents Asp, Leu, Asn, Glu, Ala, Leu, Ser, or ψ;
A22 represents Phe, or ψ;
A23 represents Val, Ile, or ψ;
A24 represents Asn, Asp, Glu, Ala, Gln, Arg, Asn, Asp, Lys, Lys-Ac, or ψ;
A25 represents Trp, or ψ;
A26 represents Leu, Aib, Iva, or ψ;
A27 represents Leu, Glu, Ser, Lys, Val, Ile, or ψ;
A28 represents Ala, Ser, Arg, Leu, Met, Lys, Lys-Ac, or ψ;
A29 represents Gln, Gly, Ala, Thr, Ser, Lys, Aib, ψ, or deletion;
A30 represents Lys, Arg, Gly, Pro, ψ, or deletion;
A31 represents Phe, Pro, Gly, ψ, or deletion;
A32 represents Lys, Ser, Gly, ψ, or deletion;
A33 represents Lys, Ser, Gly, ψ, or deletion;
A34 represents Lys, Gly, Ala, Gln, ψ, or deletion;
A35 represents Asp, Ala, Ser, Pro, Glu, ψ, or deletion;
A36 represents Trp, Pro, Gly, ψ, or deletion;
A37 represents Lys, Pro, Gly, ψ, or deletion;
A38 represents His, Pro, Ser, Gly, ψ, or deletion;
A39 represents Asn, Lys, Gly, Gln, Ser, ψ, or deletion;
A40 represents Ile, Arg, Lys, Ser, ψ, or deletion;
A41 represents Ile, Thr, ψ, or deletion;
A42 represents Gln, Gly, ψ, or deletion;
A43 represents ψ, or deletion;
wherein ψ is a residue independently selected from Lys, Arg, Orn, and Cys and wherein the side chain of said residue is substituted, and with the proviso that the GIP receptor agonist peptide is not a peptide having an amino acid sequence of SEQ ID NOs: 4 to 569 disclosed in PCT Application No. PCT/JP2018/013540, and wherein, one or two amino acids selected from A8 to A42 optionally represent Lys(R), and R represents a substituent group, or a salt thereof.

(10). In some embodiments, the present disclosure provides a method for treating emesis in a subject, for example, in a subject in need thereof. The method comprises administering a therapeutically effective amount of a GIP receptor agonist peptide, or a salt thereof, said peptide having at least 80% sequence identity to a peptide having the formula (III): P¹-A1-A2-A3-Gly-Thr-Phe-Ile-Ser-Asp-Tyr-A11-Ile-A13-A14-A15-A16-A17-His-Gln-A20-Asp-Phe-Val-A24-Trp-Leu-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent;

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, di-Br-Tyr;
A2 represents Ala, or Aib;
A3 represents Glu, or Pro;
A11 represents Ser, A5c, or A6c;
A13 represents Ala, or Aib;
A14 represents Met, Leu or Nle;
A15 represents Asp or Glu;
A16 represents Lys, Ala, or Lys(R);
A17 represents Ile, or Lys(R);
A20 represents Gln, or Lys(R);
A24 represents Asn, or Asp;
A27 represents Leu or Lys(R)
A28 represents Ala or Lys(R)
A29 represents Gln or Lys(R)
A30 represents Lys, Pro, or Lys(R);
A31 represents Phe, Pro, or deletion;
A32 represents Lys, Ser, or deletion;
A33 represents Lys, Ser, or deletion;
A34 represents Asn, Ala, Gly, or deletion;
A35 represents Asp, Pro, Ala, or deletion;
A36 represents Trp, Pro, or deletion;
A37 represents Lys, Pro, Lys(R), or deletion;
A38 represents His, Pro, Ser, or deletion;
A39 represents Asn, Ser, or deletion;
A40 represents Ile, Lys(R), or deletion;
A41 represents Ile, Thr, or deletion;
A42 represents Gln, or deletion;
A43 represents ψ, or deletion;
wherein Lys(R) is a Lys residue and (R) represents a substituent group, or salt thereof, with the proviso that the GIP receptor agonist peptide is not a peptide having an amino acid sequence of SEQ ID NOs: 4 to 569 disclosed in PCT Application No. PCT/JP2018/013540, and wherein, one or two amino acids selected from A8 to A42 optionally represent Lys(R), and the (R) in Lys(R) represents a substituent group, or a salt thereof.

(11). A method of treating emesis in a subject, the method comprising administering a therapeutically effective amount of a GIP receptor agonist peptide, or a salt thereof, said peptide having an amino acid sequence of at least 80%, 85%, 90%, 95%, 96%, 97%, 98%, 99%, or 100% amino acid sequence identity to any one of SEQ ID NOs: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37.

(12). In some embodiments, the present disclosure provides a method for treating emesis in a subject, for example, in a subject in need thereof. The method comprises administering a therapeutically effective amount of a GIP receptor agonist peptide, or a salt thereof, said peptide having at least 80% sequence identity to a peptide having the formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

(13). In some embodiments, the present disclosure provides a method for treating emesis in a subject, for example, in a subject in need thereof. The method consisting of administering a therapeutically effective dose of a monotherapy, the monotherapy consisting essentially of a GIP receptor agonist peptide, or a salt thereof, said peptide having at least 80% sequence identity to a peptide having the formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

(14). In some embodiments, the present disclosure provides a method for treating emesis in a subject, for example, in a subject in need thereof. The method consists of administering a therapeutically effective dose of a monotherapy, the monotherapy consisting essentially of a GIP receptor agonist peptide, or a salt thereof, said peptide having at least 80% sequence identity to a peptide having the formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

(15). A method for treating emesis in a non-type 2 diabetes mellitus subject, the method comprising administering a therapeutically effective amount of a GIP receptor agonist peptide, or a salt thereof, said GIP receptor agonist peptide having an amino acid sequence of formula (I): P¹-A1-A2-A3-A4-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or

is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, or des-amino-Phe, or des-amino-Tyr;
A2 represents Ala, Aib, D-Ala, Gly, Ser, or Sar;
A3 represents Glu or Pro;
A4 represents Gly, or Ser;
A5 represents Thr, D-Iva, Glu, Iva, or Ser;
A6 represents Phe, Iva, Val, Ala, Aib, Cha, or α-methyl-Leu;
A7 represents Ile, Lys, Ala, Aib, Cha, D-Leu, Ile, Thr, Arg, or Val;
A8 represents Ser, Ala, ψ, or Aib;
A9 represents Asp, Leu, ψ, Phe, Glu, or Gln;
A10 represents Tyr, Leu, Ser, Cha, or ψ;
A11 represents Ser, Aib, A5c, A6c, D-Iva, or Iva;
A12 represents Ile, Lys, Glu, Asp, Ala, Aib, Lys-Ac, Ser, α-methyl-Phe, or ψ;
A13 represents Ala, Aib, Tyr, D-Iva, ψ, Gln, Leu, Glu, or Iva;
A14 represents Met, Nle, α-methyl-Leu, Leu, or ψ;
A15 represents Asp, Glu, Lys, Ser, Tyr, ψ, or Asn;
A16 represents Lys, Ala, Ser, Glu, Arg, Aib, Lys-Ac, or ψ;
A17 represents Ile, Lys, Arg, Aib, Gln, Glu, Lys-Ac, or ψ;
A18 represents His, Arg, Ala, Aib, D-Iva, Phe, Iva, Leu, Ser, Trp, or ψ;
A19 represents Gln, Lys, Glu, Ala, Val, Ser, Aib, Arg, or ψ;
A20 represents Gln, Lys, Ala, His, Arg, Aib, Asp, Gly, or ψ;
A21 represents Asp, Leu, Asn, Asp, Glu, Ala, Leu, Ser, Aib, or ψ;
A22 represents Phe, α-methyl-Phe, Naphthyl-Ala, Asn, Ala, Trp, or ψ;
A23 represents Val, Ile, or ψ;
A24 represents Asn, Asp, Glu, Ala, Aib, Gln, Glu, Lys, Lys-Ac, Leu, Nle, Arg, Ser, or ψ;
A25 represents Trp, Tyr, Glu, Phe, Arg, α-methyl-Phe, or ψ;
A26 represents Leu, Aib, Iva, Leu, Nle, or ψ;
A27 represents Leu, Glu, Ser, Lys, Val, Ile, Nle, or ψ;
A28 represents Ala, Ser, Arg, Leu, Met, Lys, Lys-Ac, Aib, or ψ;
A29 represents Gln, Gly, Ala, Thr, Ser, Lys, Aib, ψ, or deletion;
A30 represents Lys, Arg, Gly, Pro, Glu, Lys-Ac, ψ, or deletion;
A31 represents Phe, Pro, Gly, ψ, or deletion;
A32 represents Lys, Ser, Gly, ψ, or deletion;
A33 represents Lys, Ser, Gly, Ile, Ser, ψ, or deletion;
A34 represents Asn, Ala, Gly, Gln, ψ, or deletion;
A35 represents Asp, Ala, Ser, Pro, Glu, ψ, or deletion;
A36 represents Trp, Pro, Gly, ψ, or deletion;
A37 represents Lys, Pro, Gly, ψ, or deletion;
A38 represents His, Pro, Gly, Ser, ψ, or deletion;
A39 represents Asn, Ser, Gly, Asn, Lys, Gln, ψ, or deletion;
A40 represents Ile, Arg, Glu, Lys, Ser, Lys-Ac, Arg, ψ, or deletion;
A41 represents Ile, Thr, Gly, ψ, or deletion;
A42 represents Gln, Gly, ψ, or deletion;
A43 represents ψ, or deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

(16). In some embodiments, the present disclosure provides a method for treating emesis in a non-type 2 diabetes mellitus subject. The method comprises administering a therapeutically effective amount of a GIP receptor agonist peptide, or a salt thereof, said GIP receptor agonist peptide having an amino acid sequence of formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

(17). In some embodiments, the present disclosure provides a method for treating emesis in a subject, for example, in a subject in need thereof. The method comprises administering a therapeutically effective amount of a GIP receptor agonist peptide, or a salt thereof, said peptide having the formula (V): P¹-Tyr-A2-A3-Gly-Thr-Phr-A7-Ser-Asp-A10-Ser-A12-Ala-A14-A15-A16-A17-A18-A19-A20-A21-Phe-A23-A24-A25-Leu-A27-A28-A29-A30-A31-A32-A33-A34-A35-A36-A37-A38-A39-A40-A41-A42-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib, Ala, Gly, Ser, D-Ala, or N-methyl-Ser;
A3 represents Glu, Asp, or Gln
A7 represents Ile, or Ser, Thr;
A10 represents Tyr, Leu, or Ser;
A12 represents Ile, Lys or Lys®;
A14 represents Ile, Leu, or Met;
A15 represents Asp, or Glu;
A16 represents Lys, Ser, Glu or ψ
A17 represents Ile, Lys, Gln, or ψ
Alb represents Ala, Arg, or His;
A19 represents Gln, Ala, Lys, or Glu,
A20 represents Gln, Arg, or Lys, His or Ala,
A21 represents Asp, or Ala;
A23 represents Val, or Ile;
A24 represents Asn, Gln, or Asp;
A25 represents Trp, or Thr;
A27 represents Leu, Glu, Ser, Lys, or Val;
A28 represents Ala, Ser or Arg;
A29 represents Gln, Aib, Gly, Ala, Thr, Ser or Lys;
A30 represents Lys Gly or ψ;
A31 represents Gly, Pro Gly-OH, or deletion;
A32 represents Lys, Ser, a deletion;
A33 represents Lys, Ser or a deletion;
A34 represents Asn, Gly, or a deletion;
A35 represents Asp, Ala, or a deletion;
A36 represents Trp, Pro or a deletion;
A37 represents Lys, Pro or a deletion;
A38 represents His, Pro or a deletion;
A39 represents Asn, Ser, or a deletion;
A40 represents Ile, or a deletion;
A41 represents Thr, or a deletion;
A42 represents Gln or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

(18). In some embodiments, the present disclosure provides a method for treating emesis in a subject, for example, in a subject in need thereof. The method comprises administering a therapeutically effective amount of a GIP receptor agonist peptide, or a salt thereof, said peptide having the formula (VI): P¹-Tyr-A2-Glu-Gly-Thr-Phr-A7-Ser-Asp-Thr-Ser-Ile-A13-A14-Asp-Lys-Ile-A18-Gln-A20-A21-A22-Val-A24-Trp-Leu-A27-Ala-A29-A30-A31-A32-A33-A34-A35-A36-A37-A38-A39-A40-A41-A42-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib or Ala:
A7 represents Ile, or s, Thr;
A13 represents Aib, or Ala;
A14 represents Leu, or Met;
Alb represents Ala, or His;
A20 represents Gln, Arg, or ψ;
A21 represents Asp, or Ala;
A22 represents Phe, Naphthyl-Ala, or αMePhe;
A24 represents Asn, or Gln;
A27 represents Leu or Ile;
A29 represents Gln, Aib, or Gly;
A30 represents Lys or Gly;
A31 represents Gly or Pro;
A32 represents Lys, or Ser;
A33 represents Lys, or Ser;
A34 represents Asn, Gly, or Gln;
A35 represents Asp, Glu, or Ala;
A36 represents Trp, or Pro;
A37 represents Lys, or Pro;
A38 represents His, or Pro;
A39 represents Asn, Gln, or Ser;
A40 represents Ile, or a deletion;
A41 represents Ile, Thr, or a deletion;
A42 represents Gln or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

(19). In some embodiments, the present disclosure provides a method for treating emesis in a subject, for example, in a subject in need thereof. The method comprises administering a therapeutically effective amount of a GIP receptor agonist peptide, or a salt thereof, said peptide having the formula (VII): P¹-Tyr-A2-Glu-Gly-Thr-Phr-A7-Ser-Asp-Thr-Ser-A12-A13-A14-Asp-A16-A17-A18-Gln-A20-A21-Phe-Val-A24-Trp-A26-A27-Ala-A29-A30-A31-A32-A33-A34-A35-A36-A37-A38-A39-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib or Ala:
A7 represents Ile, Leu, or Thr;
A12 represents Ile or Leu;
A13 represents Aib, or Ala;
A14 represents Leu, Met or ψ;
A16 represents Lys or Arg;
A17 represents Ile or Aib;
A18 represents Ala, His, or ψ;
A20 represents Gln, or Aib;
A21 represents Asp, Ala, Asn or ψ;
A24 represents Asn, Glu, or Gln;
A26 represents Leu or Ile;
A27 represents Leu or Ile;
A29 represents Gln or ψ;
A30 represents Lys, Arg, Ser or Gln;
A31 represents Gly or Pro or a deletion;
A32 represents Ser or a deletion;
A33 represents Ser or a deletion;
A34 represents Gly, or a deletion;
A35 represents Ala or a deletion;
A36 represents Pro or a deletion;
A37 represents Pro or a deletion;
A38 represents Pro or a deletion;
A39 represents Ser or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

(20). The method according to any one or more of embodiments (1-19), wherein the (R) group in Lys(R) represents X-L-, L represents a bivalent linker comprising PEG and/or two or more amino acids, and X represents a substituent group, or a salt thereof.

(21). The method according to any one or more of embodiments (1-19), wherein the (R) group in Lys(R) represents X-L-, L represents a bond or a bivalent substituent group, and X represents an optionally substituted hydrocarbon group, or a salt thereof.

(22). The method according to any one or more of embodiments (1-19) represented by formula: Ac-YAEGTFISDYSIAMDKIHQQDFVNWLLAQKGKKNDWKHNIIQ-OH (SEQ ID NO: 4), or a salt thereof.

(23). The method according to any one or more of embodiments (1-19) represented by formula: Y(D-Ala)EGTFISDYSIAMDKIHQQDFVNWLLAQKGKKNDW(Lys(R))HNIIQ-NH₂ (SEQ ID NO: 5), or a salt thereof, wherein (R) is a substituent group for example, a palmitoyl acyl fatty acid.

(24). The method according to any one or more of embodiments (1-19) represented by formula: YAEGTFISDYSIAMD(LysR)IHQQDFVNWLLAQKGKKNDWKHNIIQ-NH₂ (SEQ ID NO: 6), or a salt thereof.

(25). The method according to any one or more of embodiments (1-19) represented by formula: YD-AlaEGTFISDYSIAMDKIHQQDFVNWLLAQK-OH (SEQ ID NO: 7), or a salt thereof.

(26). The method according to any one or more of embodiments (1-19) represented by formula: YAibEGTFISDYSIAMDKK(g-Glu-C16)HQQDFVNWLLAQKGKKNDWKHNIIQ-OH (SEQ ID NO: 8), or a salt thereof.

(27). The method according to any one or more of embodiments (1-19) represented by formula: FAibEGTFISDYX*IA(Nle)DKIHQQDFVNWLLAQKGKKNDWKHNITQ-OH (X* is 1-NH,1-cyclopentane carboxylic acid; SEQ ID NO: 12).

(28). The method according to any one or more of embodiments (1-19) represented by formula: (3,5-diBr Tyr)AibEGTFISDYSIAib(Nle)DKIHQQDFVNWLLAQ KGKKNDWKHNITQ-OH (SEQ ID NO: 11).

(29). The method according to any one or more of embodiments (1-19) represented by formula:

(SEQ ID NO: 10)
Ac-(D-Tyr)AEGTFISDYSIAMDAIHQ
QDFVNWLLAQ(Lys(R))-NH₂,

wherein (R) represents a substituent group, for example (R) is a palmitoyl acyl fatty acid group.

(30). The method according to any one or more of embodiments (1-19) represented by formula: YAPGTFISDYSIAMDKIHQQDFVNWLLAQKPSSGAPPPS-NH₂ (SEQ ID NO: 15).

(31). The method according to any one or more of embodiments (1-19) represented by formula:

(SEQ ID NO: 14)
Y(D-A1a)EGTFISDYSIAMDKIHQQ
DFVNWLLAQKPSSGAPPPS-NH₂.

(32). The method according to any one or more of embodiments (1-19) represented by formula:

(SEQ ID NO: 9)
YAibEGTFTSDYSIYMEKEAV(Lys(R))EFIAWLVKG-OH.

(33). The method according to any one or more of embodiments (1-19) represented by formula:

(SEQ ID NO: 13)
YAibEGTFTSDYSIYLDKQAAAibEFVN
WLLAGGPSSGAPPPS(Lys(R))-NH₂.

(34). The method according to any one or more of embodiments (1-19) represented by formula:

(SEQ ID NO: 16)
YD-A1aEGTFISDYSIAMD(Lys(R))IH
QQDFVNWLLAQKGKKNDWKHNIIQ-NH₂

(35). The method according to any one or more of embodiments (1-19) represented by formula:

(SEQ ID NO: 17)
YD-A1aEGTFISDYSIAMDKIHQQDFVNW
LLAQKGKKNDWKHNIIQ-NH₂.

(36). The method according to any one or more of embodiments (1-19) represented by formula:

(SEQ ID NO: 18)
YAibEGTFTSDYSIYMEKEAVREFIAWLVKGGPSSGAPPPS
(Lys(R))-NH₂.

(37). The method according to any one or more of embodiments (1-19) represented by formula:

(SEQ ID NO: 19)
YAibEGTFTSDYSIAibLDKIAQRAFVQWLIAAibKGKKQEWKHQITQ
(Lys(R))-NH₂.

(38). The method according to any one or more of embodiments (1-19) represented by formula:

(SEQ ID NO: 20)
YAibEGTFTSDYSTYLDKQAAAibEFVNWLLAGGPSSGAPPPS-
(Lys(R))-NH₂.

(39). The method according to any one or more of embodiments (1-19) represented by formula:

(SEQ ID NO: 21)
YAibEGTFTSDYSIAibLDKIAQ(Lys(R))AFVQWLIAGGPSSGAPP
PS-NH₂.

(40). The method according to any one or more of embodiments (1-19) represented by formula:

(SEQ ID NO: 22)
YAibEGTFISDYSIAMDKIHQQDFVNWLLAQKGKKNDWKHNITQ
(Lys(R))-OH.

(41). The method according to any one or more of embodiments (1-19) represented by formula:

(SEQ ID NO: 23)
YAibEGTFISDYSIELDK(Lys(R))AAQAFIEWLLAQGPSSGAPPPS-
NH₂.

(42). The method according to any one or more of embodiments (1-19) represented by formula:

(SEQ ID NO: 24)
YAibEGTFISDYSIELDKIAAQDFIEWLLAGPSSGAPPPS(Lys(R))-
NH₂.

(43). The method according to any one or more of embodiments (1-19) represented by formula:

(SEQ ID NO: 25)
YAibEGTFISDYS(Lys(R))ELDKIAQRAFIEWLLAQGPSSGAPPPS-
NH₂.

(44). The method according to any one or more of embodiments (1-19) represented by formula:

(SEQ ID NO: 26)
YAibEGTFISDYSIELEK(Lys(R))AQRAFVEWLLAQGPSSGAPPPS-
NH₂.

(45). The method according to any one or more of embodiments (1-19) represented by formula: YAibEGTFISDYSIALDKIHQQ(Lys(R))FVNWLLAQRG-OH (SEQ ID NO: 27).

(46). The method according to any one or more of embodiments (1-19) represented by formula: YAibEGTFISDYSIALDKI(Lys(R))QQDFVNWLLAQRG-OH (SEQ ID NO: 28).

(47). The method according to any one or more of embodiments (1-19) represented by formula: YAibEGTFISDYSIALDK(Lys(R))HQQDFVNWLLAQRG-OH (SEQ ID NO: 29).

(48). The method according to any one or more of embodiments (1-19) represented by formula: YAibEGTFISDYSIALDK(Lys(R))HQQDFVNWLLAQR-NH₂ (SEQ ID NO: 30).

(49). The method according to any one or more of embodiments (1-19) represented by formula: YAibEGTFISDYSIALDKIHQQ(Lys(R))FVNWLLAQR-NH₂ (SEQ ID NO: 31).

(50). The method according to any one or more of embodiments (1-19) represented by formula:

(SEQ ID NO: 32)
YAibEGTFISDYSIALDKIHQQ(Lys(R))FVNWLLAQRPSSGAPPPS-
NH₂.

(51). The method according to any one or more of embodiments (1-19) represented by formula:

(SEQ ID NO: 33)
YAibEGTFISDYSIALDKI(Lys(R))QQDFVNWLLAQRPSSGAPPPS-
NH₂.

(52). The method according to any one or more of embodiments (1-19) represented by formula: YAibEGTFISDYSIALDKIHQQDFVNWLLAQ(Lys(R))G-OH (SEQ ID NO: 34).

(53). The method according to any one or more of embodiments (1-19) represented by formula: YAibEGTFISDYS(Lys(R))ALDKIHQQDFVNWLLAQRG-OH (SEQ ID NO: 35).

(54). The method according to any one or more of embodiments (1-19) represented by formula:

(SEQ ID NO: 36)
YAibEGTFTSDYSIAibLDKIAQ(Lys(R))AFVQWLIAGGPSSGAPP
PS-NH₂.

(55). The method according to any one or more of embodiments (1-19) represented by formula: YAibEGTFTSDYSIAibLDKIAQ(Lys(R))AFVQWLIAGGPSSGAPPPS-NH₂ (SEQ ID NO: 37), wherein in each of the embodiments 22-55, (R) in (Lys(R)) represents a substituent.

(56). The method according to any one of embodiments (1-19), wherein the GIP receptor agonist peptide of the present disclosure has at least 80%, or at least 85%, or at least 90%, or at least 95%, or at least 96%, or at least 97%, or at least 98%, or at least 99%, or 100% sequence identity to any GIP receptor agonist peptide as defined by formulas (I), (II), (III), (IV), (V), (VI), or (VII).

(57). The method according to any one of embodiments (1-19), wherein the GIP receptor agonist peptide of the present disclosure has 100% sequence identity to any GIP receptor agonist peptide as defined by formulas (I), (II), (III), (IV), (V), (VI), or (VII).

(58). A pharmaceutical composition for use in the treatment of emesis, the pharmaceutical composition comprising a GIP receptor agonist peptide of any one of embodiments (1-57).

(59). The method according to any one of embodiments (1-58), wherein the GIP receptor agonist peptide or a salt thereof, is formulated into a medicament, for use in the treatment of emesis in a subject in need thereof.

(60). The method according to embodiment (56), wherein the GIP receptor agonist peptide has a selectivity ratio of GIPR/GLPR activation (expressed as the ratio of (GLP1R EC₅₀/GIPR EC₅₀) of greater than 10, or greater than 100, or greater than 1,000, or greater than 10,000, or greater than 100,000, or in some embodiments, preferably greater than 100-1,000,000, or more.

(61). The method according to any one of embodiments (1-57), wherein the GIP receptor agonist peptide or medicament or pharmaceutical composition is administered to treat emesis as a monotherapy.

(62). The method according to any one of embodiments (1-58), wherein the emesis is nausea and/or vomiting.

(63). The method according to any one of embodiments (1-58), wherein the nausea and/or vomiting is a result of cyclic vomiting syndrome or chemotherapy.

(64). The method of any one of embodiments (62-63), wherein the subject is a non-type 2 diabetes mellitus subject.

(65). The method according to any one of embodiments (1-64), wherein the emesis is delayed emesis or anticipatory emesis.

(66). The method of embodiment (1), the use of embodiment (58), the medicament of embodiment (59), or the method of embodiment (62), where the vomiting or the nausea is caused by one or more conditions or causes selected from the following (1) to (10):

(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome, chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium;
(6) Vomiting and nausea caused by a vestibular disorder such as motion sickness or dizziness
(7) Opioid withdrawal;
(8) Pregnancy including hyperemesis gravidarium;
(9) A vestibular disorder such as motion sickness or dizziness; and
(10) A physical injury causing local, systemic, acute or chronic pain.

This description includes all or part of the contents as disclosed in the description, claims and/or drawings of PCT Application No. PCT/JP2018/013540, incorporated herein by reference in its entirety.

All publications, patents, and patent applications cited herein are incorporated herein by reference in their entirety.

In some embodiments, the GIP receptor agonist peptides of formulas (I)-(VII) selectively activate the GIP receptor and demonstrates an antiemetic action in vivo.

It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Other features and advantages of the disclosure will be apparent from the following Detailed Description, the drawings, and the claims.

### BRIEF DESCRIPTION OF THE FIGURES

FIGS. 1A-1E illustrate the anti-emetic effect of various GIP receptor agonist peptides in an in-vivo morphine induced emesis model in male ferret when morphine is administered 0.5 h after drug administration, according to the present disclosure.

FIGS. 2A-2C illustrate the anti-emetic effect of various GIP receptor agonist peptides in an in-vivo morphine induced emesis model in male ferret when morphine is administered 4 h after drug administration, according to the present disclosure.

FIGS. 3A-3C illustrate the anti-emetic effect of various GIP receptor agonist peptides in an in-vivo cisplatin induced emesis model in male ferret when cisplatin is administered 0.5 h after drug administration, according to the present disclosure.

FIGS. 4A-4D illustrate the anti-emetic effect of various GIP receptor agonist peptides in an in-vivo morphine induced emesis model in male ferret when morphine is administered 4 h after drug administration, according to the present disclosure.

FIGS. 5A-5D illustrate the anti-emetic effect of various GIP receptor agonist peptides in an in-vivo morphine induced emesis model in male ferret when morphine is administered 4 h after drug administration, according to the present disclosure.

### DETAILED DESCRIPTION OF THE EMBODIMENTS

The definition of each substituent used in the present specification is described in detail in the following. Unless otherwise specified, each substituent has the following definition.

In the present specification, examples of the “halogen atom” include fluorine, chlorine, bromine and iodine.

In the present specification, examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl.

In the present specification, examples of the “optionally halogenated C₁₋₆ alkyl group” include a C₁₋₆ alkyl group optionally having 1 to 7, preferably 1 to 5, halogen atoms. Specific examples thereof include methyl, chloromethyl, difluoromethyl, trichloromethyl, trifluoromethyl, ethyl, 2-bromoethyl, 2,2,2-trifluoroethyl, tetrafluoroethyl, pentafluoroethyl, propyl, 2,2-difluoropropyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl and 6,6,6-trifluorohexyl.

In the present specification, examples of the “C₂₋₆ alkenyl group” include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl and 5-hexenyl.

In the present specification, examples of the “C₂₋₆ alkynyl group” include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and 4-methyl-2-pentynyl.

In the present specification, examples of the “C₃₋₁₀ cycloalkyl group” include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and adamantyl.

In the present specification, examples of the “optionally halogenated C₃₋₁₀ cycloalkyl group” include a C₃₋₁₀ cycloalkyl group optionally having 1 to 7, preferably 1 to 5, halogen atoms. Specific examples thereof include cyclopropyl, 2,2-difluorocyclopropyl, 2,3-difluorocyclopropyl, cyclobutyl, difluorocyclobutyl, cyclopentyl, cyclohexyl, cycloheptyl and cyclooctyl.

In the present specification, examples of the “C₃₋₁₀ cycloalkenyl group” include cyclopropenyl, cyclobutenyl, cyclopentenyl, cyclohexenyl, cycloheptenyl and cyclooctenyl.

In the present specification, examples of the “C₆₋₁₄ aryl group” include phenyl, 1-naphthyl, 2-naphthyl, 1-anthryl, 2-anthryl and 9-anthryl.

In the present specification, examples of the “C₇₋₁₆ aralkyl group” include benzyl, phenethyl, naphthylmethyl and phenylpropyl.

In the present specification, examples of the “C₁₋₆ alkoxy group” include methoxy, ethoxy, propoxy, isopropoxy, butoxy, isobutoxy, sec-butoxy, tert-butoxy, pentyloxy and hexyloxy.

In the present specification, examples of the “optionally halogenated C₁₋₆ alkoxy group” include a C₁₋₆ alkoxy group optionally having 1 to 7, preferably 1 to 5, halogen atoms. Specific examples thereof include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

In the present specification, examples of the “C₃₋₁₀ cycloalkyloxy group” include cyclopropyloxy, cyclobutyloxy, cyclopentyloxy, cyclohexyloxy, cycloheptyloxy and cyclooctyloxy.

In the present specification, examples of the “C₁₋₆ alkylthio group” include methylthio, ethylthio, propylthio, isopropylthio, butylthio, sec-butylthio, tert-butylthio, pentylthio and hexylthio.

In the present specification, examples of the “optionally halogenated C₁₋₆ alkylthio group” include a C₁₋₆ alkylthio group optionally having 1 to 7, preferably 1 to 5, halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

In the present specification, examples of the “C₁₋₆ alkyl-carbonyl group” include acetyl, propanoyl, butanoyl, 2-methylpropanoyl, pentanoyl, 3-methylbutanoyl, 2-methylbutanoyl, 2,2-dimethylpropanoyl, hexanoyl and heptanoyl.

In the present specification, examples of the “optionally halogenated C₁₋₆ alkyl-carbonyl group” include a C₁₋₆ alkyl-carbonyl group optionally having 1 to 7, preferably 1 to 5, halogen atoms. Specific examples thereof include acetyl, chloroacetyl, trifluoroacetyl, trichloroacetyl, propanoyl, butanoyl, pentanoyl and hexanoyl.

In the present specification, examples of the “C₁₋₆ alkoxy-carbonyl group” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl and hexyloxycarbonyl.

In the present specification, examples of the “C₆₋₁₄ aryl-carbonyl group” include benzoyl, 1-naphthoyl and 2-naphthoyl.

In the present specification, examples of the “C₇₋₁₆ aralkyl-carbonyl group” include phenylacetyl and phenylpropionyl.

In the present specification, examples of the “5- to 14-membered aromatic heterocyclylcarbonyl group” include nicotinoyl, isonicotinoyl, thenoyl and furoyl.

In the present specification, examples of the “3- to 14-membered non-aromatic heterocyclylcarbonyl group” include morpholinylcarbonyl, piperidinylcarbonyl and pyrrolidinylcarbonyl.

In the present specification, examples of the “mono- or di-C₁₋₆ alkyl-carbamoyl group” include methylcarbamoyl, ethylcarbamoyl, dimethylcarbamoyl, diethylcarbamoyl and N-ethyl-N-methylcarbamoyl.

In the present specification, examples of the “mono- or di-C₇₋₁₆ aralkyl-carbamoyl group” include benzylcarbamoyl and phenethylcarbamoyl.

In the present specification, examples of the “C₁₋₆ alkylsulfonyl group” include methylsulfonyl, ethylsulfonyl, propylsulfonyl, isopropylsulfonyl, butylsulfonyl, sec-butylsulfonyl and tert-butylsulfonyl.

In the present specification, examples of the “optionally halogenated C₁₋₆ alkylsulfonyl group” include a C₁₋₆ alkylsulfonyl group optionally having 1 to 7, preferably 1 to 5, halogen atoms. Specific examples thereof include methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl and hexylsulfonyl.

In the present specification, examples of the “C₆₋₁₄ arylsulfonyl group” include phenylsulfonyl, 1-naphthylsulfonyl and 2-naphthylsulfonyl.

In the present specification, examples of the “substituent” include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

In the present specification, examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

In the present specification, examples of the “optionally substituted hydrocarbon group” include a hydrocarbon group optionally having substituent(s) selected from the following substituent group A.

[substituent group A]
(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl)(C₁₋₆ alkyl-carbonyl)amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tat-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group and
(62) a C₆₋₁₄ aryl group.

The number of the above-mentioned substituents in the “optionally substituted hydrocarbon group” is, for example, 1 to 5, preferably 1 to 3. When the number of the substituents is two or more, the respective substituents may be the same or different.

In the present specification, examples of the “heterocyclic group” (including “heterocyclic group” of “optionally substituted heterocyclic group”) include (i) an aromatic heterocyclic group, (ii) a non-aromatic heterocyclic group and (iii) a 7- to 10-membered bridged heterocyclic group, each containing, as a ring-constituting atom besides carbon atom, 1 to 4 hetero atoms selected from a nitrogen atom, a sulfur atom and an oxygen atom.

In the present specification, examples of the “aromatic heterocyclic group” (including “5- to 14-membered aromatic heterocyclic group”) include a 5- to (preferably 5- to 10-membered) aromatic heterocyclic group containing, as a ring-constituting atom besides carbon atom, 1 to 4 hetero atoms selected from a nitrogen atom, a sulfur atom and an oxygen atom.

Preferable examples of the “aromatic heterocyclic group” include 5- or 6-membered monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like; and 8- to 14-membered fused polycyclic (preferably bi or tricyclic) aromatic heterocyclic groups such as benzothiophenyl, benzofuranyl, benzimidazolyl, benzoxazolyl, benzisoxazolyl, benzothiazolyl, benzisothiazolyl, benzotriazolyl, imidazopyridinyl, thienopyridinyl, furopyridinyl, pyrrolopyridinyl, pyrazolopyridinyl, oxazolopyridinyl, thiazolopyridinyl, imidazopyrazinyl, imidazopyrimidinyl, thienopyrimidinyl, furopyrimidinyl, pyrrolopyrimidinyl, pyrazolopyrimidinyl, oxazolopyrimidinyl, thiazolopyrimidinyl, pyrazolotriazinyl, naphtho[2,3-b]thienyl, phenoxathiinyl, indolyl, isoindolyl, 1H-indazolyl, purinyl, isoquinolyl, quinolyl, phthalazinyl, naphthyridinyl, quinoxalinyl, quinazolinyl, cinnolinyl, carbazolyl, β-carbolinyl, phenanthridinyl, acridinyl, phenazinyl, phenothiazinyl, phenoxazinyl and the like.

In the present specification, examples of the “non-aromatic heterocyclic group” (including “3- to 14-membered non-aromatic heterocyclic group”) include a 3- to 14-membered (preferably 4- to 10-membered) non-aromatic heterocyclic group containing, as a ring-constituting atom besides carbon atom, 1 to 4 hetero atoms selected from a nitrogen atom, a sulfur atom and an oxygen atom.

Preferable examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and 9- to 14-membered fused polycyclic (preferably bi or tricyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

In the present specification, preferable examples of the “7- to 10-membered bridged heterocyclic group” include quinuclidinyl and 7-azabicyclo[2.2.1]heptanyl.

In the present specification, examples of the “nitrogen-containing heterocyclic group” include a “heterocyclic group” containing at least one nitrogen atom as a ring-constituting atom.

In the present specification, examples of the “optionally substituted heterocyclic group” include a heterocyclic group optionally having substituent(s) selected from the aforementioned substituent group A.

The number of the substituents in the “optionally substituted heterocyclic group” is, for example, 1 to 3. When the number of the substituents is two or more, the respective substituents may be the same or different.

In the present specification, examples of the “acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀ cycloalkyl group, a C₃₋₁₀ cycloalkenyl group, a C₆₋₁₄ aryl group, a C₇₋₁₆ aralkyl group, a 5- to 14-membered aromatic heterocyclic group and a 3- to 14-membered non-aromatic heterocyclic group, each of which optionally has 1 to 3 substituents selected from a halogen atom, an optionally halogenated C₁₋₆ alkoxy group, a hydroxy group, a nitro group, a cyano group, an amino group and a carbamoyl group”.

Examples of the “acyl group” (also referred to as “Ac”) also include a hydrocarbon-sulfonyl group, a heterocyclylsulfonyl group, a hydrocarbon-sulfinyl group and a heterocyclylsulfinyl group.

In some embodiments, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Preferable examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

In the present specification, examples of the “optionally substituted amino group” include an amino group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from substituent group A”.

Preferable examples of the optionally substituted amino group include an amino group, a mono- or di-(optionally halogenated C₁₋₆ alkyl)amino group (e.g., methylamino, trifluoromethylamino, dimethylamino, ethylamino, diethylamino, propylamino, dibutylamino), a mono- or di-C₂₋₆ alkenylamino group (e.g., diallylamino), a mono- or di-C₃₋₁₀ cycloalkylamino group (e.g., cyclopropylamino, cyclohexylamino), a mono- or di-C₆₋₁₄ arylamino group (e.g., phenylamino), a mono- or di-C₇₋₁₆ aralkylamino group (e.g., benzylamino, dibenzylamino), a mono- or di-(optionally halogenated C₁₋₆alkyl)-carbonylamino group (e.g., acetylamino, propionylamino), a mono- or di-C₆₋₁₄ aryl-carbonylamino group (e.g., benzoylamino), a mono- or di-C₇₋₁₆ aralkyl-carbonylamino group (e.g., benzylcarbonylamino), a mono- or di-5- to 14-membered aromatic heterocyclylcarbonylamino group (e.g., nicotinoylamino, isonicotinoylamino), a mono- or di-3- to 14-membered non-aromatic heterocyclylcarbonylamino group (e.g., piperidinylcarbonylamino), a mono- or di-C₁₋₆ alkoxy-carbonylamino group (e.g., tert-butoxycarbonylamino), a 5- to 14-membered aromatic heterocyclylamino group (e.g., pyridylamino), a carbamoylamino group, a (mono- or di-C₁₋₆ alkyl-carbamoyl)amino group (e.g., methylcarbamoylamino), a (mono- or di-C₇₋₁₆ aralkyl-carbamoyl)amino group (e.g., benzylcarbamoylamino), a C₁₋₆ alkylsulfonylamino group (e.g., methylsulfonylamino, ethylsulfonylamino), a C₆₋₁₄ arylsulfonylamino group (e.g., phenylsulfonylamino), a (C₁₋₆ alkyl)(C₁₋₆ alkyl-carbonyl)amino group (e.g., N-acetyl-N-methylamino) and a (C₁₋₆ alkyl)(C₆₋₁₄ aryl-carbonyl)amino group (e.g., N-benzoyl-N-methylamino).

In the present specification, examples of the “optionally substituted carbamoyl group” include a carbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Preferable examples of the optionally substituted carbamoyl group include a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl, cyclohexylcarbamoyl), a mono- or di-C₆₋₁₄ aryl-carbamoyl group (e.g., phenylcarbamoyl), a mono- or di-C₇₋₁₆ aralkyl-carbamoyl group, a mono- or di-C₁₋₆ alkyl-carbonyl-carbamoyl group (e.g., acetylcarbamoyl, propionylcarbamoyl), a mono- or di-C₆₋₁₄ aryl-carbonyl-carbamoyl group (e.g., benzoylcarbamoyl) and a 5- to 14-membered aromatic heterocyclylcarbamoyl group (e.g., pyridylcarbamoyl).

In the present specification, examples of the “optionally substituted thiocarbamoyl group” include a thiocarbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Preferable examples of the optionally substituted thiocarbamoyl group include a thiocarbamoyl group, a mono- or di-C₁₋₆ alkyl-thiocarbamoyl group (e.g., methylthiocarbamoyl, ethylthiocarbamoyl, dimethylthiocarbamoyl, diethylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a mono- or di-C₁₋₆ alkyl-carbonyl-thiocarbamoyl group (e.g., acetylthiocarbamoyl, propionylthiocarbamoyl), a mono- or di-C₆₋₁₄ aryl-carbonyl-thiocarbamoyl group (e.g., benzoylthiocarbamoyl) and a 5- to 14-membered aromatic heterocyclylthiocarbamoyl group (e.g., pyridylthiocarbamoyl).

In the present specification, examples of the “optionally substituted sulfamoyl group” include a sulfamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Preferable examples of the optionally substituted sulfamoyl group include a sulfamoyl group, a mono- or di-C₁₋₆ alkyl-sulfamoyl group (e.g., methylsulfamoyl, ethylsulfamoyl, dimethylsulfamoyl, diethylsulfamoyl, N-ethyl-N-methylsulfamoyl), a mono- or di-C₂₋₆ alkenyl-sulfamoyl group (e.g., diallylsulfamoyl), a mono- or di-C₃₋₁₀ cycloalkyl-sulfamoyl group (e.g., cyclopropylsulfamoyl, cyclohexylsulfamoyl), a mono- or di-C₆₋₁₄ aryl-sulfamoyl group (e.g., phenylsulfamoyl), a mono- or di-C₇₋₁₆ aralkyl-sulfamoyl group (e.g., benzylsulfamoyl, phenethylsulfamoyl), a mono- or di-C₁₋₆ alkyl-carbonyl-sulfamoyl group (e.g., acetylsulfamoyl, propionylsulfamoyl), a mono- or di-C₆₋₁₄ aryl-carbonyl-sulfamoyl group (e.g., benzoylsulfamoyl) and a 5- to 14-membered aromatic heterocyclylsulfamoyl group (e.g., pyridylsulfamoyl).

In the present specification, examples of the “optionally substituted hydroxy group” include a hydroxyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from substituent group A”.

Preferable examples of the optionally substituted hydroxy group include a hydroxy group, a C₁₋₆ alkoxy group, a C₂₋₆ alkenyloxy group (e.g., allyloxy, 2-butenyloxy, 2-pentenyloxy, 3-hexenyloxy), a C₃₋₁₀ cycloalkyloxy group (e.g., cyclohexyloxy), a C₆₋₁₄ aryloxy group (e.g., phenoxy, naphthyloxy), a C₇₋₁₆ aralkyloxy group (e.g., benzyloxy, phenethyloxy), a C₁₋₆ alkyl-carbonyloxy group (e.g., acetyloxy, propionyloxy, butyryloxy, isobutyryloxy, pivaloyloxy), a C₆₋₁₄ aryl-carbonyloxy group (e.g., benzoyloxy), a C₇₋₁₆ aralkyl-carbonyloxy group (e.g., benzylcarbonyloxy), a 5- to 14-membered aromatic heterocyclylcarbonyloxy group (e.g., nicotinoyloxy), a 3- to 14-membered non-aromatic heterocyclylcarbonyloxy group (e.g., piperidinylcarbonyloxy), a C₁₋₆ alkoxy-carbonyloxy group (e.g., tert-butoxycarbonyloxy), a 5- to 14-membered aromatic heterocyclyloxy group (e.g., pyridyloxy), a carbamoyloxy group, a C₁₋₆ alkyl-carbamoyloxy group (e.g., methylcarbamoyloxy), a C₇₋₁₆ aralkyl-carbamoyloxy group (e.g., benzylcarbamoyloxy), a C₁₋₆ alkylsulfonyloxy group (e.g., methylsulfonyloxy, ethylsulfonyloxy) and a C₆₋₁₄ arylsulfonyloxy group (e.g., phenylsulfonyloxy).

In the present specification, examples of the “optionally substituted sulfanyl group” include a sulfanyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group and a 5- to 14-membered aromatic heterocyclic group, each of which optionally has 1 to 3 substituents selected from substituent group A” and a halogenated sulfanyl group.

Preferable examples of the optionally substituted sulfanyl group include a sulfanyl (—SH) group, a C₁₋₆ alkylthio group, a C₂₋₆ alkenylthio group (e.g., allylthio, 2-butenylthio, 2-pentenylthio, 3-hexenylthio), a C₃₋₁₀ cycloalkylthio group (e.g., cyclohexylthio), a C₆₋₁₄ arylthio group (e.g., phenylthio, naphthylthio), a C₇₋₁₆ aralkylthio group (e.g., benzylthio, phenethylthio), a C₁₋₆ alkyl-carbonylthio group (e.g., acetylthio, propionylthio, butyrylthio, isobutyrylthio, pivaloylthio), a C₆₋₁₄ aryl-carbonylthio group (e.g., benzoylthio), a 5- to 14-membered aromatic heterocyclylthio group (e.g., pyridylthio) and a halogenated thio group (e.g., pentafluorothio).

In the present specification, examples of the “optionally substituted silyl group” include a silyl group optionally having “1 to 3 substituents selected from a C₁₋₆ alkyl group, a C₂₋₆ alkenyl group, a C₃₋₁₀ cycloalkyl group, a C₆₋₁₄ aryl group and a C₇₋₁₆ aralkyl group, each of which optionally has 1 to 3 substituents selected from substituent group A”.

Preferable examples of the optionally substituted silyl group include a tri-C₁₋₆ alkylsilyl group (e.g., trimethylsilyl, tert-butyl(dimethyl)silyl).

For descriptions of amino acid residues, the following conventions may be exemplified: Asp≤D=Aspartic Acid; Ala=A=Alanine; Arg=R=Arginine; Asn=N=Asparagine; Cys=C=Cysteine; Gly=G=Glycine; Glu=E=Glutamic Acid; Gln=Q=Glutamine; His=H=Histidine; Ile=I=Isoleucine; Leu=L=Leucine; Lys=K=Lysine; Met=M=Methionine; Phe=F=Phenylalanine; Pro=P=Proline; Ser=S=Serine; Thr=T=Threonine; Trp=W=Tryptophan; Tyr=Y=Tyrosine; and Val=V=Valine.

Also for convenience, and readily known to one skilled in the art, the following abbreviations or symbols are used to represent the moieties, reagents and the like used in present disclosure:

(D)-Tyr—D-tyrosine;

(D)-Ala—D-Alanine

DesNH₂-Tyr—desaminotyrosine;

(D)-Phe—D-phenylalanine;

DesNH₂-Phe—desaminophenylalanine;

(D)-Trp—D-tryptophan;

(D)₃Pya—D-3-pyridylalanine;

2-Cl-(D)Phe—D-2-chlorophenylalanine;

3-Cl-(D)Phe—D-3-chlorophenylalanine;

4-Cl-(D)Phe—D-4-chlorophenylalanine;

2-F-(D)Phe—D-2-fluorophenylalanine;

3-F(D)Phe—D-3-fluorophenylalanine;

3,5-DiF-(D)Phe—D-3,5-difluorophenylalanine;

3,4,5-TriF-(D)Phe—D-3,4,5-trifluorophenylalanine;

D-Iva—D-Isovaline

SSA—succinimidyl succinamide;

PEG—polyethylene glycol;

PEGₘ—(methoxy)polyethylene glycol;

PEGₘ(12,000)—(methoxy)polyethylene glycol having a molecular weight of about 12 kD;

PEGₘ(20,000)—(methoxy)polyethylene glycol having a molecular weight of about 20 kD;

PEGₘ(30,000)—(methoxy)polyethylene glycol having a molecular weight of about 30 kD;

Fmoc—9-fluorenylmethyloxycarbonyl;

DMF—dimethylformamide;

DIPEA—N,N-diisopropylethylamine;

TFA—trifluoroacetic acid;

HOBT—N-hydroxybenzotriazole;

BOP—benzotriazol-1-yloxy-tris-(dimethylamino)phosphoniumhexafluorophosphate;

HBTU—2-(1H-benzotriazole-1-yl)-1,1,3,3-tetramethyluroniumhexafluorophosphate;

NMP—N-methyl-pyrrolidone;

FAB-MS fast atom bombardment mass spectrometry;

ES-MS—electro spray mass spectrometry.

Abu: α-aminobutyric acid;

Acc: 1-amino-1-cyclo(C₃-C₉)alkyl carboxylic acid;

A3c: 1-amino-1-cyclopropane carboxylic acid;

A4c: 1-amino-1-cyclobutanecarboxylic acid;

A5c: 1-amino-1-cyclopentanecarboxylic acid;

A6c: 1-amino-1-cyclohexanecarboxylic acid;

Act: 4-amino-4-carboxytetrahydropyran;

Ado: 12-aminododecanoic acid;

Aib: .alpha.-aminoisobutyric acid;

Aic: 2-aminoindan-2-carboxylic acid;

β-Ala: beta-alanine;

Amp: 4-amino-phenylalanine;

Apc: 4-amino-4-carboxypiperidine;

hArg: homoarginine;

Aun: 11-aminoundecanoic acid;

Ava: 5-aminovaleric acid;

Cha: β-cyclohexylalanine;

Dhp: 3,4-dehydroproline;

Dmt: 5,5-dimethylthiazolidine-4-carboxylic acid;

Gaba: γ-aminobutyric acid;

4Hppa: 3-(4-hydroxyphenyl)propionic acid;

3Hyp: 3-hydroxyproline;

4Hyp: 4-hydroxyproline;

hPro: homoproline;

4Ktp: 4-ketoproline;

Nle: norleucine;

NMe-Tyr: N-methyl-tyrosine;

1Nal or 1-Nal: β-(1-naphthyl)alanine;

2 Nal or 2-Nal: β-(2-naphthyl)alanine;

Nva: norvaline;

Orn: ornithine;

2OEGgE-C15 diacid is (ethylene glycol)₂-C15 diacid;

2OEGgE-C20 diacid is (ethylene glycol)₂-C20 diacid;

2OEGgE-Eda is (ethylene glycol)₂-C20 diacid;

2xOEG-gGlu-Oda is (ethylene glycol)₂-gGlu-C18 diacid

gEgEgE-Palmitoyl is (gamma-glutamic acid)₃-palmitoyl

2Pal or 2-Pal: β-(2-pyridinyl)alanine;

3Pal or 3-Pal: β-(3-pyridinyl)alanine;

4Pal or 4-Pal: β-(4-pyridinyl)alanine;

Pen: penicillamine;

(3,4,5F)Phe: 3,4,5-trifluorophenylalanine;

(2,3,4,5,6)Phe: 2,3,4,5,6-pentafluorophenylalanine;

Psu: N-propylsuccinimide;

Iva: Isovaline;

Sar: Sarcosine;

Taz: β-(4-thiazolyl)alanine;

3Thi: β-(3-thienyl)alanine;

Thz: thioproline;

Tic: tetrahydroisoquinoline-3-carboxylic acid;

Tle: tert-leucine;

Act: acetonitrile;

Boc: tert-butyloxycarbonyl;

BSA: bovine serum albumin;

DCM: dichloromethane;

DTT: dithiothrieitol;

ESI: electrospray ionization;

Fmoc: 9-fluorenylmethyloxycarbonyl;

HBTU: 2-(1H-benzotriazole-1-yl)-1,1,3,3-tetramethyluronium hexafluorophosphate;

HPLC: high performance liquid chromatography;

IBMX: isobutylmethylxanthine;

LC-MS: liquid chromatography-mass spectrometry;

Mtt: methyltrityl;

NMP: N-methylpyrrolidone;

5K PEG: polyethylene glycol, which may include other functional groups or moieties such as a linker, and which is either linear or branched as defined hereinbelow, with an average total molecular weight of about 5,000

10K PEG: polyethylene glycol, which may include other functional groups or moieties such as a linker, and which is either linear or branched as defined hereinbelow, with an average total molecular weight of about 10,000

20K PEG: polyethylene glycol, which may include other functional groups or moieties such as a linker, and which is either linear or branched as defined hereinbelow, with an average total molecular weight of about 20,000

30K PEG: polyethylene glycol, which may include other functional groups or moieties such as a linker, and which is either linear or branched as defined hereinbelow, with an average total molecular weight of about 30,000

40K PEG: polyethylene glycol, which may include other functional groups or moieties such as a linker, and which is either linear or branched as defined hereinbelow, with an average total molecular weight of about 40,000

50K PEG: polyethylene glycol, which may include other functional groups or moieties such as a linker, and which is either linear or branched as defined hereinbelow, with an average total molecular weight of about 50,000

60K PEG: polyethylene glycol, which may include other functional groups or moieties such as a linker, and which is either linear or branched as defined hereinbelow, with an average total molecular weight of about 60,000

tBu: tert-butyl;

TIS: triisopropylsilane;

Trt: trityl;

Z: benzyloxycarbonyl;

As used herein, “PEG moiety” refers to polyethylene glycol (PEG) or a derivative thereof, for example (methoxy)polyethylene glycol (PEGₘ).

As used herein, “PEGylated peptide” refers to a peptide wherein at least one amino acid residue, for example, Lys, or Cys has been conjugated with a PEG moiety. By “conjugated”, it is meant that the PEG moiety is either directly linked to said residue or is linked to the residue via a spacer moiety, for example a cross-linking agent. When said conjugation is at a lysine residue, that lysine residue is referred to herein as “PEGylated Lys”. A peptide that is conjugated to only one PEG moiety is said to be “mono-PEGylated”.

As used herein, “Lys-PEG” and “Lys-PEGₘ” refer respectively to lysine residues which have been conjugated with PEG and PEGₘ. “Lys(epsilon-SSA-PEGₘ)” refers to a lysine residue wherein the epsilon-amino group has been cross-linked with PEGₘ using a suitably functionalized SSA. “Lys(epsilon-SSA-PEGₘ(12,000))” refers to a lysine residue wherein the epsilon-amino group has been cross-linked with PEGₘ(12,000) using a suitably-functionalized SSA; “Lys(epsilon-SSA-PEGₘ(20,000))” refers to a lysine residue wherein the epsilon-amino group has been cross-linked with PEGₘ(20,000) using a suitably-functionalized SSA; and “Lys(epsilon-SSA-PEGₘ(30,000))” refers to a lysine residue wherein the epsilon-amino group has been cross-linked with PEGₘ(30,000) using a suitably-functionalized SSA.

In the present specification, the term “human native GIP peptide” refers to the naturally occurring human GIP peptide. This human native GIP peptide (42 amino acids) has an amino acid sequence: YAEGTFISDYSIAMDKIHQ QDFVNWLLAQKGKKNDWKHNITQ (SEQ ID NO: 1) and is the functionally active molecule from the parent precursor described in National Center for Biotechnology Information (NCBI) Reference Sequence: NP_004114.1; REFSEQ: accession NM_004123.2 (SEQ ID NO: 2) obtained from the mRNA sequence of human gastric inhibitory polypeptide (GIP), mRNA; ACCESSION: NM_004123; VERSION; NM_004123.2 (SEQ ID NO: 3).

“Percent (%) amino acid sequence identity” with respect to a reference polypeptide sequence is defined as the percentage of amino acid residues in a candidate polypeptide sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “treatment” (and variations such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of a condition, alleviation of symptoms, diminishment of any direct or indirect pathological consequences of the condition or treatment, preventing emesis, i.e. by preventing the occurrence of symptoms in whole or in part associated with a condition or side-effects known to accompany a specific treatment, decreasing the rate of progression, amelioration or palliation of the symptoms associated with emesis, such as nausea and/or vomiting, and remission or improved prognosis. In some embodiments, GIP receptor agonist peptides of the disclosure are used to inhibit or delay development of emesis, ie. nausea or vomiting or to slow the progression of emesis or the symptoms associated with emesis, or to prevent, delay or inhibit the development of emesis, nausea and/or vomiting related to the treatment of a different disease being actively treated.

By “reduce” or “inhibit” is meant the ability to cause an overall decrease of 20%, 30%, 40%, 50%, 60%, 70%, 75%, 80%, 85%, 90%, 95%, or greater. In some embodiments, reduce or inhibit can refer to a relative reduction compared to a reference (e.g., reference level of biological activity (e.g., the number of episodes of nausea and/or vomiting after administration to a subject of a prescribed amount of chemotherapy, for example, a prescribed dose of a chemotherapeutic agent that is known to cause emesis). In some embodiments, reduce or inhibit can refer to the relative reduction of a side effect (i.e. nausea and/or vomiting) associated with a treatment for a condition or disease.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. MoI. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)). One illustrative example of an algorithm that is suitable for determining percent sequence identity and sequence similarity is the BLAST algorithm, which is described by Altschul et al. (J. MoI. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

Variants can also be synthetic, recombinant, or chemically modified polynucleotides or polypeptides isolated or generated using methods well known in the art. Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine.

“Conservative amino acid substitutions” as referenced herein result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

As used herein, the term “selectivity” of a molecule for a first receptor relative to a second receptor refers to the following ratio: EC₅₀ of the molecule at the second receptor divided by the EC₅₀ of the molecule at the first receptor. For example, a molecule that has an EC₅₀ of 1 nM at a first receptor and an EC₅₀ of 100 nM at a second receptor has 100-fold selectivity for the first receptor relative to the second receptor.

As is understood by one skilled in the art, reference to “about” a value or parameter herein includes (and describes) embodiments that are directed to that value or parameter per se or that have a variance plus or minus of that value ranging from less than 10%, or less than 9%, or less than 8%, or less 7%, or less than 6%, or less than 5%, or less than 4%, or less than 3%, or less than 2%, or less than 1%, or less than 0.1% than the stated value. For example, description referring to “about X” includes description of “X”.

It is understood that aspect and embodiments of the disclosure described herein include “consisting” and/or “consisting essentially” of aspects and embodiments. As used herein, the singular form “a”, “an”, and “the” includes plural references unless indicated otherwise.

### A. GIP RECEPTOR AGONIST PEPTIDES

In various embodiments of the present disclosure, methods are provided for the treatment of emesis in a subject in need thereof. In various embodiments, the methods provide administration of a therapeutically effective amount of a GIP receptor agonist peptide to the subject.

As used herein, GIP receptor agonist peptides of the present disclosure refer to peptides that preferentially bind to GIP receptors compared to other receptors, such as GLP receptors. In some embodiments, an exemplary GIP receptor agonist peptide of the present disclosure are GIP receptor agonist peptides that have a selectivity ratio as defined as the ratio of (EC₅₀ GLP1R/EC₅₀ GIPR) greater than 10, or greater than 100, or greater than 1,000, or greater than 10,000, or greater than 100,000. Preferably, an exemplary GIP receptor agonist peptide is a GIP receptor agonist peptide when the peptide has a selectivity ratio of (EC₅₀ GLP1R/EC₅₀ GIPR) of greater than 100, or from about 100-1,000,000 or more.

In some embodiments, a method is provided to prevent or treat emesis in a subject. A GIP receptor agonist peptide, or a salt thereof, for use in the methods, compositions and medicaments exemplified herein for use in the prevention and/or treatment of emesis, include a GIP receptor agonist peptide that does not have an amino acid sequence as set forth in SEQ ID NO: 1.

In some embodiments, the GIP receptor agonist peptide, or a salt thereof, includes a GIP receptor agonist peptide that does not have an amino acid sequence as set forth in any one of SEQ ID NO: 4 to 569 disclosed in PCT Application No. PCT/JP2018/013540.

A GIP receptor agonist peptide, or a salt thereof, for use in the methods, compositions and medicaments exemplified herein, include a GIP receptor agonist peptide having at least 80% sequence identity to amino acid sequence of the native human GIP peptide having an amino acid sequence as set forth in SEQ ID NO: 1, with the proviso that the peptide does not have an amino sequence as set forth in SEQ ID NO: 1.

A GIP receptor agonist peptide, or a salt thereof, for use in the methods, compositions and medicaments exemplified herein, include a GIP receptor agonist peptide having 29 or 30 amino acids, or a salt thereof.

A GIP receptor agonist peptide, or a salt thereof, for use in the methods, compositions and medicaments exemplified herein, include a GIP receptor agonist peptide, or a salt thereof, wherein the GIP receptor agonist peptide has at least 80%, at least 85%, at least 90%, at least 95%, at least 96%, or at least 97%, sequence identity to amino acid sequence of the native human GIP peptide having an amino acid sequence as set forth in SEQ ID NO: 1, with the proviso that the peptide does not have an amino sequence as set forth in SEQ ID NO: 1.

A GIP receptor agonist peptide, or a salt thereof, for use in the methods, compositions and medicaments exemplified herein, include a GIP receptor agonist peptide having 29 or 30 amino acids, or a salt thereof, wherein the GIP receptor agonist peptide has at least 80% sequence identity to amino acid sequence 1-29 or 1-30 of the native human GIP peptide having an amino acid sequence as set forth in SEQ ID NO: 1.

A GIP receptor agonist peptide, or a salt thereof, for use in the methods, compositions and medicaments exemplified herein, include a GIP receptor agonist peptide having at least 80% sequence identity to amino acid sequence 1-29 or 1-30 of the native human GIP peptide having an amino acid sequence as set forth in SEQ ID NO: 1, with the proviso that the GIP receptor agonist peptide does not have an amino acid sequence as set forth in any 29 or 30 amino acid peptide provided in SEQ ID NO: 4 to 569 disclosed in PCT Application No. PCT/JP2018/013540.

A GIP receptor agonist peptide, or a salt thereof, for use in the methods, compositions and medicaments exemplified herein, include a GIP receptor agonist peptide having the formula (I): P¹-A1-A2-A3-A4-A5-A6-A7-A8-A9-A10-A11-A12-A13-A14-A15-A16-A17-A18-A 19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or

is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, or des-amino-Phe, or des-amino-Tyr;
A2 represents Ala, Aib, D-Ala, Gly, Ser, or Sar;
A3 represents Glu or Pro;
A4 represents Gly, or Ser;
A5 represents Thr, D-Iva, Glu, Iva, or Ser;
A6 represents Phe, Iva, Val, Ala, Aib, Cha, or α-methyl-Leu;
A7 represents Ile, Lys, Ala, Aib, Cha, D-Leu, Ile, Thr, Arg, or Val;
A8 represents Ser, Ala, ψ, or Aib;
A9 represents Asp, Leu, ψ, Phe, Glu, or Gln;
A10 represents Tyr, Leu, Ser, Cha, or ψ;
A11 represents Ser, Aib, A5c, A6c, D-Iva, or Iva;
A12 represents Ile, Lys, Glu, Asp, Ala, Aib, Lys-Ac, Ser, α-methyl-Phe, or ψ;
A13 represents Ala, Aib, Tyr, D-Iva, ψ, Gln, Leu, Glu, or Iva;
A14 represents Met, Nle, α-methyl-Leu, Leu, or ψ;
A15 represents Asp, Glu, Lys, Ser, Tyr, ψ, or Asn;
A16 represents Lys, Ala, Ser, Glu, Arg, Aib, Lys-Ac, or ψ;
A17 represents Ile, Lys, Arg, Aib, Gln, Glu, Lys-Ac, or ψ;
A18 represents His, Arg, Ala, Aib, D-Iva, Phe, Iva, Leu, Ser, Trp, or ψ;
A19 represents Gln, Lys, Glu, Ala, Val, Ser, Aib, Arg, or ψ;
A20 represents Gln, Lys, Ala, His, Arg, Aib, Asp, Gly, or ψ;
A21 represents Asp, Leu, Asn, Asp, Glu, Ala, Leu, Ser, Aib, or ψ;
A22 represents Phe, α-methyl-Phe, Naphthyl-Ala, Asn, Ala, Trp, or ψ;
A23 represents Val, Ile, or ψ;
A24 represents Asn, Asp, Glu, Ala, Aib, Gln, Glu, Lys, Lys-Ac, Leu, Nle, Arg, Ser, or ψ;
A25 represents Trp, Tyr, Glu, Phe, Arg, α-methyl-Phe, or ψ;
A26 represents Leu, Aib, Iva, Leu, Nle, or ψ;
A27 represents Leu, Glu, Ser, Lys, Val, Ile, Nle, or ψ;
A28 represents Ala, Ser, Arg, Leu, Met, Lys, Lys-Ac, Aib, or ψ;
A29 represents Gln, Gly, Ala, Thr, Ser, Lys, Aib, ψ, or deletion;
A30 represents Lys, Arg, Gly, Pro, Glu, Lys-Ac, ψ, or deletion;
A31 represents Phe, Pro, Gly, ψ, or deletion;
A32 represents Lys, Ser, Gly, ψ, or deletion;
A33 represents Lys, Ser, Gly, Ile, Ser, ψ, or deletion;
A34 represents Asn, Ala, Gly, Gln, ψ, or deletion;
A35 represents Asp, Ala, Ser, Pro, Glu, ψ, or deletion;
A36 represents Trp, Pro, Gly, ψ, or deletion;
A37 represents Lys, Pro, Gly, ψ, or deletion;
A38 represents His, Pro, Gly, Ser, ψ, or deletion;
A39 represents Asn, Ser, Gly, Asn, Lys, Gln, ψ, or deletion;
A40 represents Ile, Arg, Glu, Lys, Ser, Lys-Ac, Arg, ψ, or deletion;
A41 represents Ile, Thr, Gly, ψ, or deletion;
A42 represents Gln, Gly, ψ, or deletion;
A43 represents ψ, or deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1

In some related embodiments, at least one of R^{A1}, R^{A2}, and R^{A3} is a methyl (Me) group.

A GIP receptor agonist peptide, or a salt thereof, for use in the methods, compositions and medicaments exemplified herein, include a GIP receptor agonist peptide, or a salt thereof, the peptide having the formula (II): P¹-A1-A2-A3-Gly-Thr-A6-A7-Ser-A9-A10-A11-A12-A13-A14-A15-A16-A17-A18-A19-A20-A21-A22-A23-A24-Trp-Leu-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent;

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, des-amino-Phe, or des-amino-Tyr;
A2 represents Ala, Aib, D-Ala, Gly, Ser, or Sar;
A3 represents Glu, or Pro;
A6 represents Phe, Iva, or Val;
A7 represents Ile, Lys, Thr, or Val;
A9 represents Asp, Leu, or Phe;
A10 represents Tyr, or ψ;
A11 represents Ser, A5c, Leu, Aib, or Cha;
A12 represents Ile, Lys, Glu, Asp, or ψ;
A13 represents Ala, Aib, Tyr, D-Iva, Gln, Leu, or Glu;
A14 represents Met, Nle, Leu, or ψ;
A15 represents Asp, Glu, Lys, Ser, or Tyr;
A16 represents Lys, Ala, Ser, Glu, Arg, or ψ;
A17 represents Ile, Lys, Arg, Aib, Gln, Ile, Glu, or ψ;
A18 represents His, Ala, or ψ;
A19 represents Gln, Lys, Glu, Ala, Val, Ser, or ψ;
A20 represents Gln, Lys, Ala, His, Arg, Aib, or ψ;
A21 represents Asp, Leu, Asn, Glu, Ala, Leu, Ser, or ψ;
A22 represents Phe, or ψ;
A23 represents Val, Ile, or ψ;
A24 represents Asn, Asp, Glu, Ala, Gln, Arg, Asn, Asp, Lys, Lys-Ac, or ψ;
A25 represents Trp, or ψ;
A26 represents Leu, Aib, Iva, or ψ;
A27 represents Leu, Glu, Ser, Lys, Val, Ile, or ψ;
A28 represents Ala, Ser, Arg, Leu, Met, Lys, Lys-Ac, or ψ;
A29 represents Gln, Gly, Ala, Thr, Ser, Lys, Aib, ψ, or deletion;
A30 represents Lys, Arg, Gly, Pro, ψ, or deletion;
A31 represents Phe, Pro, Gly, ψ, or deletion;
A32 represents Lys, Ser, Gly, ψ, or deletion;
A33 represents Lys, Ser, Gly, ψ, or deletion;
A34 represents Lys, Gly, Ala, Gln, ψ, or deletion;
A35 represents Asp, Ala, Ser, Pro, Glu, ψ, or deletion;
A36 represents Trp, Pro, Gly, ψ, or deletion;
A37 represents Lys, Pro, Gly, ψ, or deletion;
A38 represents His, Pro, Ser, Gly, ψ, or deletion;
A39 represents Asn, Lys, Gly, Gln, Ser, ψ, or deletion;
A40 represents Ile, Arg, Lys, Ser, ψ, or deletion;
A41 represents Ile, Thr, ψ, or deletion;
A42 represents Gln, Gly, ψ, or deletion;
A43 represents w, or deletion;
wherein ψ is a residue independently selected from Lys, Arg, Orn, and Cys and wherein the side chain of said residue is substituted, and with the proviso that the GIP receptor agonist peptide is not a peptide having an amino acid sequence of SEQ ID NOs: 4 to 569 disclosed in PCT Application No. PCT/JP2018/013540, and wherein, one or two amino acids selected from A8 to A42 optionally represent Lys(R), and R represents a substituent group, or a salt thereof.

A GIP receptor agonist peptide, or a salt thereof, for use in the methods, compositions and medicaments exemplified herein, include a GIP receptor agonist peptide, or a salt thereof, said peptide having at least 80% sequence identity to a peptide having the formula (III): P¹-A1-A2-A3-Gly-Thr-Phe-Ile-Ser-Asp-Tyr-A1l-Ile-A13-A14-A15-A16-A17-His-Gln-A20-Asp-Phe-Val-A24-Trp-Leu-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent;

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, di-Br-Tyr;
A2 represents Ala, or Aib;
A3 represents Glu, or Pro;
A11 represents Ser, A5c, or A6c;
A13 represents Ala, or Aib;
A14 represents Met, Leu or Nle;
A15 represents Asp or Glu;
A16 represents Lys, Ala, or Lys(R);
A17 represents Ile, or Lys(R);
A20 represents Gln, or Lys(R);
A24 represents Asn, or Asp;
A27 represents Leu or Lys(R)
A28 represents Ala or Lys(R)
A29 represents Gln or Lys(R)
A30 represents Lys, Pro, or Lys(R);
A31 represents Phe, Pro, or deletion;
A32 represents Lys, Ser, or deletion;
A33 represents Lys, Ser, or deletion;
A34 represents Asn, Ala, Gly, or deletion;
A35 represents Asp, Pro, Ala, or deletion;
A36 represents Trp, Pro, or deletion;
A37 represents Lys, Pro, Lys(R), or deletion;
A38 represents His, Pro, Ser, or deletion;
A39 represents Asn, Ser, or deletion;
A40 represents Ile, Lys(R), or deletion;
A41 represents Ile, Thr, or deletion;
A42 represents Gln, or deletion;
A43 represents ψ, or deletion;
wherein Lys(R) is a Lys residue and (R) represents a substitutent group, or salt thereof, with the proviso that the GIP receptor agonist peptide is not a peptide having an amino acid sequence of SEQ ID NOs: 4 to 569 disclosed in PCT Application No. PCT/JP2018/013540, and wherein, one or two amino acids selected from A8 to A42 optionally represent Lys(R), and the (R) in Lys(R) represents a substituent group, or a salt thereof.

In some embodiments, an illustrative GIP receptor agonist peptide, or a salt thereof, for use in the methods, compositions and medicaments exemplified herein, include a GIP receptor agonist peptide, or a salt thereof, having at least 80%, at least 85%, at least 90%, at least 95%, at least 96%, at least 97%, at least 98%, or at least 100% sequence identity to a GIP receptor agonist peptide having an amino acid sequence of SEQ ID NOs: 4, 5, 6, 7, 8, 9, 10, 11, 12, 13, 14, 15, 16, 17, 18, 19, 20, 21, 22, 23, or 24-37.

In some embodiments, a GIP receptor agonist peptide, or a salt thereof, for use in the methods, compositions and medicaments exemplified herein, include a GIP receptor agonist peptide, or a salt thereof having at least 80% sequence identity to a peptide having the formula (IV): P¹-A1-A2-A3-Gly-A5-A6-A7-A8-A9-A10-A11-A12-A13-A14-A15-A 16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

In some embodiments, a GIP receptor agonist peptide, or a salt thereof, for use in the methods, compositions and medicaments exemplified herein, include a GIP receptor agonist peptide, or a salt thereof, for use in a monotherapy, the monotherapy consisting essentially of a GIP receptor agonist peptide, or a salt thereof having at least 80% sequence identity to a peptide having the formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

In some embodiments, a GIP receptor agonist peptide, or a salt thereof, for use in the methods, compositions and medicaments exemplified herein, include a GIP receptor agonist peptide, or a salt thereof, for use in a monotherapy, the monotherapy consisting essentially of a GIP receptor agonist peptide, or a salt thereof having at least 80% sequence identity to a peptide having the formula (IV): P¹-A1-A2-A3-Gly-A5-A6-A7-A8-A9-A10-A11-A 12-A 13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

In some embodiments, a GIP receptor agonist peptide, or a salt thereof, for use in the methods, compositions and medicaments exemplified herein, include a GIP receptor agonist peptide, or a salt thereof, for use in preventing and/or treating emesis in a subject who is not a type-2 diabetes mellitus subject, or a subject who is not taking concurrently a type-2 diabetes mellitus medication, said GIP receptor agonist peptide has an amino acid sequence of formula (I): P¹-A1-A2-A3-A4-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or

is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, or des-amino-Phe, or des-amino-Tyr;
A2 represents Ala, Aib, D-Ala, Gly, Ser, or Sar;
A3 represents Glu or Pro;
A4 represents Gly, or Ser;
A5 represents Thr, D-Iva, Glu, Iva, or Ser;
A6 represents Phe, Iva, Val, Ala, Aib, Cha, or α-methyl-Leu;
A7 represents Ile, Lys, Ala, Aib, Cha, D-Leu, Ile, Thr, Arg, or Val;
A8 represents Ser, Ala, ψ, or Aib;
A9 represents Asp, Leu, ψ, Phe, Glu, or Gln;
A10 represents Tyr, Leu, Ser, Cha, or ψ;
A11 represents Ser, Aib, A5c, A6c, D-Iva, or Iva;
A12 represents Ile, Lys, Glu, Asp, Ala, Aib, Lys-Ac, Ser, α-methyl-Phe, or ψ;
A13 represents Ala, Aib, Tyr, D-Iva, ψ, Gln, Leu, Glu, or Iva;
A14 represents Met, Nle, α-methyl-Leu, Leu, or ψ;
A15 represents Asp, Glu, Lys, Ser, Tyr, ψ, or Asn;
A16 represents Lys, Ala, Ser, Glu, Arg, Aib, Lys-Ac, or ψ;
A17 represents Ile, Lys, Arg, Aib, Gln, Glu, Lys-Ac, or ψ;
A18 represents His, Arg, Ala, Aib, D-Iva, Phe, Iva, Leu, Ser, Trp, or ψ;
A19 represents Gln, Lys, Glu, Ala, Val, Ser, Aib, Arg, or ψ;
A20 represents Gln, Lys, Ala, His, Arg, Aib, Asp, Gly, or ψ;
A21 represents Asp, Leu, Asn, Asp, Glu, Ala, Leu, Ser, Aib, or ψ;
A22 represents Phe, α-methyl-Phe, Naphthyl-Ala, Asn, Ala, Trp, or ψ;
A23 represents Val, Ile, or ψ;
A24 represents Asn, Asp, Glu, Ala, Aib, Gln, Glu, Lys, Lys-Ac, Leu, Nle, Arg, Ser, or ψ;
A25 represents Trp, Tyr, Glu, Phe, Arg, α-methyl-Phe, or ψ;
A26 represents Leu, Aib, Iva, Leu, Nle, or ψ;
A27 represents Leu, Glu, Ser, Lys, Val, Ile, Nle, or ψ;
A28 represents Ala, Ser, Arg, Leu, Met, Lys, Lys-Ac, Aib, or ψ;
A29 represents Gln, Gly, Ala, Thr, Ser, Lys, Aib, ψ, or deletion;
A30 represents Lys, Arg, Gly, Pro, Glu, Lys-Ac, ψ, or deletion;
A31 represents Phe, Pro, Gly, ψ, or deletion;
A32 represents Lys, Ser, Gly, ψ, or deletion;
A33 represents Lys, Ser, Gly, Ile, Ser, ψ, or deletion;
A34 represents Asn, Ala, Gly, Gln, ψ, or deletion;
A35 represents Asp, Ala, Ser, Pro, Glu, ψ, or deletion;
A36 represents Trp, Pro, Gly, ψ, or deletion;
A37 represents Lys, Pro, Gly, ψ, or deletion;
A38 represents His, Pro, Gly, Ser, ψ, or deletion;
A39 represents Asn, Ser, Gly, Asn, Lys, Gln, ψ, or deletion;
A40 represents Ile, Arg, Glu, Lys, Ser, Lys-Ac, Arg, ψ, or deletion;
A41 represents Ile, Thr, Gly, ψ, or deletion;
A42 represents Gln, Gly, ψ, or deletion;
A43 represents ψ, or deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

In some embodiments, a GIP receptor agonist peptide, or a salt thereof, for use in the methods, compositions and medicaments exemplified herein, include a GIP receptor agonist peptide, or a salt thereof, for use in preventing and/or treating emesis in a subject who is not a type-2 diabetes mellitus subject, or a subject who is not taking concurrently a type-2 diabetes mellitus medication, said GIP receptor agonist peptide having an amino acid sequence of formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or

is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Gln, or a deletion;
A35 represents Asp, Glu, Ala, or a deletion;
A36 represents Trp, Pro, or a deletion;
A37 represents Lys, Pro, Lys(R), or a deletion;
A38 represents His, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that the GIP receptor agonist peptide is not a peptide having an amino acid sequence of SEQ ID NOs: 4 to 569 disclosed in PCT Application No. PCT/JP2018/013540, and wherein any one or two amino acids selected from A8 to A42 optionally represent Lys(R), and the (R) in Lys(R) represents a substituent group, or a salt thereof.

In some embodiments, the present disclosure provides a method for treating emesis in a subject, for example, in a subject in need thereof. The method comprises administering a therapeutically effective amount of a GIP receptor agonist peptide, or a salt thereof, said peptide having the formula (V): P¹-Tyr-A2-A3-Gly-Thr-Phr-A7-Ser-Asp-A10-Ser-A12-Ala-A14-A15-A16-A17-A18-A19-A20-A21-Phe-A23-A24-A25-Leu-A27-A28-A29-A30-A31-A32-A33-A34-A35-A36-A37-A38-A39-A40-A41-A42-P²,

wherein
P¹ represents a group represented by formula

—R^{A1},

—CO—R^{A1},

—CO—OR^{A1},

—CO—COR^{A1},

—SO—R^{A1},

—SO₂—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib, Ala, Gly, Ser, D-Ala, or N-methyl-Ser;
A3 represents Glu, Asp, or Gln
A7 represents Ile, or Ser, Thr;
A10 represents Tyr, Leu, or Ser;
A12 represents Ile, Lys or Lys®;
A 14 represents Ile, Leu, or Met;
A15 represents Asp, or Glu;
A16 represents Lys, Ser, Glu or ψ
A17 represents Ile, Lys, Gln, or ψ
A18 represents Ala, Arg, or His;
A19 represents Gln, Ala, Lys, or Glu,
A20 represents Gln, Arg, or Lys, His or Ala,
A21 represents Asp, or Ala;
A23 represents Val, or Ile;
A24 represents Asn, Gln, or Asp;
A25 represents Trp, or Thr;
A27 represents Leu, Glu, Ser, Lys, or Val;
A28 represents Ala, Ser or Arg;
A29 represents Gln, Aib, Gly, Ala, Thr, Ser or Lys;
A30 represents Lys Gly or ψ;
A31 represents Gly, Pro Gly-OH, or deletion;
A32 represents Lys, Ser, a deletion;
A33 represents Lys, Ser or a deletion;
A34 represents Asn, Gly, or a deletion;
A35 represents Asp, Ala, or a deletion;
A36 represents Trp, Pro or a deletion;
A37 represents Lys, Pro or a deletion;
A38 represents His, Pro or a deletion;
A39 represents Asn, Ser, or a deletion;
A40 represents Ile, or a deletion;
A41 represents Thr, or a deletion;
A42 represents Gln or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

In some embodiments, the present disclosure provides a method for treating emesis in a subject, for example, in a subject in need thereof. The method comprises administering a therapeutically effective amount of a GIP receptor agonist peptide, or a salt thereof, said peptide having the formula (VI): P¹-Tyr-A2-Glu-Gly-Thr-Phr-A7-Ser-Asp-Thr-Ser-Ile-A13-A14-Asp-Lys-Ile-A18-Gln-A20-A21-A22-Val-A24-Trp-Leu-A27-Ala-A29-A30-A31-A32-A33-A34-A35-A36-A37-A38-A39-A40-A41-A42-P²,

wherein
P¹ represents a group represented by formula

—R^{A1},

—CO—R^{A1},

—CO—OR^{A1},

—CO—COR^{A1},

—SO—R^{A1},

—SO₂—R^{A}

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib or Ala:
A7 represents Ile, or s, Thr;
A13 represents Aib, or Ala;
A14 represents Leu, or Met;
A18 represents Ala, or His;
A20 represents Gln, Arg, or ψ;
A21 represents Asp, or Ala;
A22 represents Phe, Naphthyl-Ala, or αMePhe;
A24 represents Asn, or Gln;
A27 represents Leu or Ile;
A29 represents Gln, Aib, or Gly;
A30 represents Lys or Gly;
A31 represents Gly or Pro;
A32 represents Lys, or Ser;
A33 represents Lys, or Ser;
A34 represents Asn, Gly, or Gln;
A35 represents Asp, Glu, or Ala;
A36 represents Trp, or Pro;
A37 represents Lys, or Pro;
A38 represents His, or Pro;
A39 represents Asn, Gln, or Ser;
A40 represents Ile, or a deletion;
A41 represents Ile, Thr, or a deletion;
A42 represents Gln or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

In some embodiments, the present disclosure provides a method for treating emesis in a subject, for example, in a subject in need thereof. The method comprises administering a therapeutically effective amount of a GIP receptor agonist peptide, or a salt thereof, said peptide having the formula (VII): P¹-Tyr-A2-Glu-Gly-Thr-Phr-A7-Ser-Asp-Thr-Ser-A12-A13-A14-Asp-A16-A17-A18-Gln-A20-A21-Phe-Val-A24-Trp-A26-A27-Ala-A29-A30-A31-A32-A33-A34-A35-A36-A37-A38-A39-P²,

wherein
P¹ represents a group represented by formula

—R^{A1},

—CO—R^{A},

—CO—OR^{A1},

—CO—COR^{A1},

—SO—R^{A1},

—SO₂—OR^{A1},

—CO—NR^{A2}R^{A3},

—SO₂—NR^{A2}R^{A3},

—C(═NR^{A1})—NR^{A2}R^{A3}, or is absent,

wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib or Ala:
A7 represents Ile, Leu, or Thr;
A12 represents Ile or Leu;
A13 represents Aib, or Ala;
A14 represents Leu, Met or ψ;
A16 represents Lys or Arg;
A17 represents Ile or Aib;
A18 represents Ala, His, or ψ;
A20 represents Gln, or Aib;
A21 represents Asp, Ala, Asn or ψ;
A24 represents Asn, Glu, or Gln;
A26 represents Leu or Ile;
A27 represents Leu or Ile;
A29 represents Gln or ψ;
A30 represents Lys, Arg, ψ, Ser or Gln;
A31 represents Gly or Pro or a deletion;
A32 represents Ser or a deletion;
A33 represents Ser or a deletion;
A34 represents Gly, or a deletion;
A35 represents Ala or a deletion;
A36 represents Pro or a deletion;
A37 represents Pro or a deletion;
A38 represents Pro or a deletion;
A39 represents Ser or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

In some embodiments, the (R) group in Lys(R) represents X-L-, L represents a bivalent linker comprising PEG and/or two or more amino acids, and X represents a substituent group, or a salt thereof. In some embodiments, (R) represents X-L-, L represents a bond or a bivalent substituent group, and X represents an optionally substituted hydrocarbon group, or a salt thereof.

In some embodiments, an illustrative GIP receptor agonist peptide for use in the methods, compositions and medicaments exemplified herein, has an amino acid sequence: Ac-YAEGTFISDYSIAMDKIHQQDFVNWLLAQKGKKNDWKHNIIQ-OH (Compound No. 1, SEQ ID NO: 4), or a salt thereof.

In some embodiments, an illustrative GIP receptor agonist peptide for use in the methods, compositions and medicaments exemplified herein, has an amino acid sequence: Y(D-Ala)EGTFISDYSIAMDKIHQQDFVNWLLAQKGKKNDW(Lys-(palmitoyl acyl fatty acid))HNIIQ-NH₂ (Compound No. 2, SEQ ID NO: 5), or a salt thereof.

In some embodiments, an illustrative GIP receptor agonist peptide for use in the methods, compositions and medicaments exemplified herein, has an amino acid sequence: YAEGTFISDYSIAMD(Lys-(palmitoyl acyl fatty acid acyl)IHQQDFVNWLLAQKG KKNDWKHNIIQ-NH₂ (Compound No. 3, SEQ ID NO: 6), or a salt thereof,

In some embodiments, an illustrative GIP receptor agonist peptide for use in the methods, compositions and medicaments exemplified herein, has an amino acid sequence: YD-AlaEGTFISDYSIAMDKIHQQDFVNWLLAQK-OH (Compound No. 4, SEQ ID NO: 7), or a salt thereof.

In some embodiments, an illustrative GIP receptor agonist peptide for use in the methods, compositions and medicaments exemplified herein, has an amino acid sequence: YAibEGTFISDYSIAMDKK(g-Glu-C₁₆)HQQDFVNWLLAQKGKKNDWKHNIIQ-OH; (Compound No. 5, SEQ ID NO: 8), or a salt thereof.

In some embodiments, an illustrative GIP receptor agonist peptide for use in the methods, compositions and medicaments exemplified herein, has an amino acid sequence: FAibEGTFISDYX*IA(Nle)DKIHQQDFVNWLLAQKGKKNDWKHNITQ-OH (X* is 1-amino,1-cyclopentane carboxylic acid; Compound No. 9, SEQ ID NO: 12), wherein R represents a substituent group, or a salt thereof.

In some embodiments, an illustrative GIP receptor agonist peptide for use in the methods, compositions and medicaments exemplified herein, has an amino acid sequence:

(Compound No. 8, SEQ ID NO: 11)
(3,5-diBr-Tyr)AibEGTFISDYSIAib(Nle)DKIHQQDFVNWLLAQ
KGKKNDWKHNITQ-OH.

In some embodiments, an illustrative GIP receptor agonist peptide for use in the methods, compositions and medicaments exemplified herein, has an amino acid sequence:

(Compound No. 12, SEQ ID NO: 15)
YAPGTFISDYSIAMDKIHQQDFVNWLLAQKPSSGAPPPS-NH₂.

In some embodiments, an illustrative GIP receptor agonist peptide for use in the methods, compositions and medicaments exemplified herein, has an amino acid sequence:

(Compound No. 11, SEQ ID NO: 14)
Y(D-Ala)EGTFISDYSIAMDKIHQQDFVNWLLAQKPSSGAPPPS-
NH₂.

In some embodiments, an illustrative GIP receptor agonist peptide for use in the methods, compositions and medicaments exemplified herein, has an amino acid sequence:

(Compound No. 7, SEQ ID NO: 10)
Ac-(D-Tyr)AEGTFISDYSIAMDAIHQQDFVNWLLAQLys-
(palmitoyl acyl fatty acid)-NH₂.

In some embodiments, an illustrative GIP receptor agonist peptide for use in the methods, compositions and medicaments exemplified herein, has an amino acid sequence:

(Compound No. 6, SEQ ID NO: 9)
YAibEGTFTSDYSIYMEKEAV(Lys-(palmitoyl acyl fatty
acid))EFIAWLVKG-OH.

In some embodiments, an illustrative GIP receptor agonist peptide for use in the methods, compositions and medicaments exemplified herein, has an amino acid sequence:

(Compound No. 10, SEQ ID NO: 13)
YAibEGTFTSDYSIYLDKQAAAibEFVNWLLAGGPSSGAPPPS(Lys-
(palmitoyl acyl fatty acid))-NH₂.

In some embodiments, an illustrative GIP receptor agonist peptide for use in the methods, compositions and medicaments exemplified herein, has an amino acid sequence:

(Compound No. 13, SEQ ID NO: 16)
YD-AlaEGTFISDYSIAMD(Lys-(OEG-gGlu)₂-Oda)IHQQDFVNW
LLAQKGKICNDWKHNIIQ-NH₂.

In some embodiments, an illustrative GIP receptor agonist peptide for use in the methods, compositions and medicaments exemplified herein, has an amino acid sequence:

(Compound No. 14, SEQ ID NO: 17)
YD-AlaEGTFISDYSIAMDKIHQQDFVNWLLAQKGKKNDWKHNIIQ-
NH₂.

In some embodiments, an illustrative GIP receptor agonist peptide for use in the methods, compositions and medicaments exemplified herein, has an amino acid sequence:

(Compound No. 15, SEQ ID NO: 18)
YAibEGTFTSDYSIYMEKEAVREFIAWLVKGGPSSGAPPPS(Lys-
Acyl)-NH₂.

In some embodiments, an illustrative GIP receptor agonist peptide for use in the methods, compositions and medicaments exemplified herein, has an amino acid sequence:

(Compound No. 16, SEQ ID NO: 19)
YAibEGTFTSDYSIAibLDKIAQRAFVQWLIAAibKGKKQEWKHQITQ
(Lys-Acyl)-NH₂.

In some embodiments, an illustrative GIP receptor agonist peptide for use in the methods, compositions and medicaments exemplified herein, has an amino acid sequence:

(Compound No. 17, SEQ ID NO: 20)
YAibEGTFTSDYSIYLDKQAAAibEFVNWLLAGGPSSGAPPPS-
(Lys-Acyl)-NH₂.

In some embodiments, an illustrative GIP receptor agonist peptide for use in the methods, compositions and medicaments exemplified herein, has an amino acid sequence:

(Compound No. 18, SEQ ID NO: 21)
YAibEGTFTSDYSIAibLDKIAQ(Lys-(ethylene glycol)₂-
C20 diacid)AFVQWLIAGGPSSGAPPPS-NH2.

In some embodiments, an illustrative GIP receptor agonist peptide for use in the methods, compositions and medicaments exemplified herein, has an amino acid sequence:

(Compound No. 19, SEQ ID NO: 22)
YAibEGTFISDYSIAMDKIHQQDFVNWLLAQKGICKNDWKHNITQ
(Lys-(gamma-glutamic acid)₃-palmitoyl)-OH.

In some embodiments, an illustrative GIP receptor agonist peptide for use in the methods, compositions and medicaments exemplified herein, has an amino acid sequence:

(Compound No. 20, SEQ ID NO: 23)
YAibEGTFISDYSIELDK(Lys-(ethylene glycol)₂-C15
diacid)AAQAFIEWLLAQGPSSGAPPPS-NH2.

In some embodiments, an illustrative GIP receptor agonist peptide for use in the methods, compositions and medicaments exemplified herein, has an amino acid sequence:

(Compound No. 21, SEQ ID NO: 24)
YAibEGTFISDYSIELDKIAAQDFIEWLLAGPSSGAPPPS(Lys-
(ethylene glycol)₂-C15 diacid)-NH2.

In some embodiments, an illustrative GIP receptor agonist peptide for use in the methods, compositions and medicaments exemplified herein, has an amino acid sequence:

(Compound No. 22, SEQ ID NO: 25)
YAibEGTFISDYS(Lys-(ethylene glycol)₂-C15 diacid)
ELDKIAQRAFIEWLLAQGPSSGAPPPS-NH2.

In some embodiments, an illustrative GIP receptor agonist peptide for use in the methods, compositions and medicaments exemplified herein, has an amino acid sequence:

(Compound No. 23, SEQ ID NO: 26)
YAibEGTFISDYSIELEK(Lys-(ethylene glycol)₂-C15
diacid)AQRAFVEWLLAQGPSSGAPPPS-NH2.

In some embodiments, an illustrative GIP receptor agonist peptide for use in the methods, compositions and medicaments exemplified herein, has an amino acid sequence:

(Compound No. 24, SEQ ID NO: 27)
YAibEGTFISDYSIALDKIHQQ(Lys-(ethylene glycol)₂-C15
diacid)FVNWLLAQRG-OH.

In some embodiments, an illustrative GIP receptor agonist peptide for use in the methods, compositions and medicaments exemplified herein, has an amino acid sequence:

(Compound No. 25, SEQ ID NO: 28)
YAibEGTFISDYSIALDKI(Lys-(ethylene glycol)₂-C15
diacid)QQDFVNWLLAQRG-OH.

In some embodiments, an illustrative GIP receptor agonist peptide for use in the methods, compositions and medicaments exemplified herein, has an amino acid sequence:

(Compound No. 26, SEQ ID NO: 29)
YAibEGTFISDYSIALDK(Lys-(ethylene glycol)₂-C15
diacid)HQQDFVNWLLAQRG-OH.

In some embodiments, an illustrative GIP receptor agonist peptide for use in the methods, compositions and medicaments exemplified herein, has an amino acid sequence:

(Compound No. 27, SEQ ID NO: 30)
YAibEGTFISDYSIALDK(Lys-(ethylene glycol)₂-C15
diacid)HQQDFVNWLLAQR-NH2.

In some embodiments, an illustrative GIP receptor agonist peptide for use in the methods, compositions and medicaments exemplified herein, has an amino acid sequence:

(Compound No. 28, SEQ ID NO: 31)
YAibEGTFISDYSIALDKIHQQ(Lys-(ethylene glycol)₂-
C15 diacid)FVNWLLAQR-NH2.

In some embodiments, an illustrative GIP receptor agonist peptide for use in the methods, compositions and medicaments exemplified herein, has an amino acid sequence:

(Compound No. 29, SEQ ID NO: 32)
YAibEGTFISDYSIALDKIHQQ(Lys-(ethylene glycol)₂-
C15 diacid)FVNWLLAQRPSSGAPPPS-NH2.

In some embodiments, an illustrative GIP receptor agonist peptide for use in the methods, compositions and medicaments exemplified herein, has an amino acid sequence:

(Compound No. 30, SEQ ID NO: 33)
YAibEGTFISDYSIALDKI(Lys-(ethylene glycol)₂-C15
diacid)QQDFVNWLLAQRPSSGAPPPS-NH2.

In some embodiments, an illustrative GIP receptor agonist peptide for use in the methods, compositions and medicaments exemplified herein, has an amino acid sequence:

(Compound No. 31, SEQ ID NO: 34)
YAibEGTFISDYSIALDKIHQQDFVNWLLAQ(Lys-(ethylene
glycol)₂-C15 diacid)G-OH.

In some embodiments, an illustrative GIP receptor agonist peptide for use in the methods, compositions and medicaments exemplified herein, has an amino acid sequence:

(Compound No. 32, SEQ ID NO: 35)
YAibEGTFISDYS(Lys-(ethylene glycol)₂-C15 diacid)
ALDKIHQQDFVNWLLAQRG-OH.

In some embodiments, an illustrative GIP receptor agonist peptide for use in the methods, compositions and medicaments exemplified herein, has an amino acid sequence:

(Compound No. 33, SEQ ID NO: 36)
YAibEGTFTSDYSIAibLDKIAQ(Lys-(ethylene glycol)₂-
C15 diacid)AFVQWLIAGGPSSGAPPPS-NH2.

In some embodiments, an illustrative GIP receptor agonist peptide for use in the methods, compositions and medicaments exemplified herein, has an amino acid sequence:

(Compound No. 34, SEQ ID NO: 37)
YAibEGTFTSDYSIAibLDKIAQ(Lys-(ethylene glycol)₂-
C20 diacid)AFVQWLIAGGPSSGAPPPS-NH2.

In each of the embodiments above, (R) in Lys(R) represents a substituent, or a salt thereof.

In various embodiments, an illustrative GIP receptor agonist peptide for use in the methods, compositions and medicaments exemplified herein has at least 80%, or at least 85%, or at least 90%, or at least 95%, or at least 96%, or at least 97%, or at least 98%, or at least 99%, or 100% sequence identity to any GIP receptor agonist peptide as defined by formulas (I), (II), (III), (IV), (V), (VI), or (VII).

In various embodiments, an illustrative GIP receptor agonist peptide for use in the methods, compositions and medicaments exemplified herein, has 100% sequence identity to any GIP receptor agonist peptide as defined by formulas (I), (II), (III), (IV), (V), (VI), or (VII).

In various embodiments, an illustrative GIP receptor agonist peptide for use in the methods, compositions and medicaments exemplified herein, has at least 80%, or at least 85%, or at least 90%, or at least 95%, or at least 96%, or at least 97%, or at least 98%, or at least 99%, or 100% sequence identity to any GIP receptor agonist peptide as defined by formulas (I), (II), (III), (IV), (V), (VI), or (VII), and wherein the GIP receptor agonist peptide is a GIP selective peptide.

With reference to the above GIP receptor agonist peptides as defined by formulas (I), (II), (III), (IV), (V), (VI), or (VII), in various embodiments, a GIP receptor agonist peptide has at least one, two or three amino acids having a bivalent substituent, covalently coupled to a side chain of an amino acid. For example, in some embodiments, a GIP receptor agonist peptide and an amino acid sequence having a side chain of at least one amino acid, or modified amino acid for example, a Cys, homocystein, Lys, Orn, Dab, Dap, or p-amine-phenylalanine residue of the GIP receptor agonist peptide being covalently attached to a substituent group. In various embodiments, a Cys, homocystein, Lys, Orn, Dab, Dap, or p-amine-phenylalanine residue of the GIP receptor agonist peptide may be covalently attached to a C6-C20 acyl fatty acid, for example, a palmitoyl acyl fatty acid, a myristoyl acyl fatty acid, and other acyl fatty acids ranging in length from C6-C20, and wherein the fatty acids may be saturated, unsaturated or partially unsaturated or partially unsaturated.

In various embodiments, a Cys, homocystein, Lys, Orn, Dab, Dap, or p-amine-phenylalanine residue of the GIP receptor agonist peptide may be covalently attached to a substituent, for example a bivalent substituent. In some embodiments, a Cys, homocystein, Lys, Orn, Dab, Dap, or p-amine-phenylalanine residue of the GIP receptor agonist peptide may be covalently attached to an (R) group. In some illustrative examples, the (R) group may be covalently linked to a side chain of a Lys amino acid. In some examples, an exemplary (R) group represents X-L-, wherein L represents a bivalent linker comprising PEG and/or two or more amino acids, and X represents a substituent group, or a salt thereof.

With reference to the above methods, compositions, medicaments described herein, when the GIP receptor agonist peptide has a Lys(R) modified amino acid, the (R) group represents X-L-, wherein L represents a bond or a bivalent substituent group, and X represents an optionally substituted hydrocarbon group, or a salt thereof.

With reference to the above methods, when the GIP receptor agonist peptide has a Lys(R) modified amino acid, the (R) group represents X-L-, wherein -L- represents -GGGGG- or -GGGGGG-, (PEG3)2, (PEG3)3, (PEG3)4, (PEG3)5, PEG(4), gGlu, (gGlu)2, (gGlu)3, (gGlu)2-PEG, AMBZ-PEG3, GABA-(PEG3)2, GABA-GGG, GG, GGG, GGGG, GGGGG, PEG(3), PEG3 or any combination of these. In some embodiments, X represents a C8-C20 diacid or dicarboxylic acid: Heda, Hepda, Oda, Ida, or Eda. In some embodiments, X represents a C8-20 acid: capryloyl, pelargonoyl, caproyl, undecanoyl, lauroyl, tridecanoyl, myristoyl, dentadecanoyl, palmoyl, margaroyl, steroyl, nonadecanoyl, eicosanoyl, heneicosanoyl or behenoyl. In some embodiments, one, two, or three amino acids from the sequence of the selected GIP receptor agonist peptide described herein, for example, from positions A8 to A42 of the GIP receptor agonist peptide optionally represent Lys(R), wherein (R) represents X-L-, wherein -L- represents -GGGGG- or -GGGGGG- and X represents Heda, Hepda, Oda, Ida, or Eda. In some embodiments, (R) is an acyl fatty acid, for example, palmitoyl or myristoyl, or (R) may be PEG(20,000) or PEG(30,000) or SSA-PEG(10,000-60,000 Da.), for example, SSA-PEG(30,000). In some embodiments, the (R) group coupled to a Lys is g-Glu-C16 or a (PEG3)2-g-Glu-C16 or (PEG3)2-g-Glu-C18. In some embodiments, the (R) group coupled to a Lys is palmitoyl or a g-Glu-Palmitoyl

In some embodiments, the (R) group attached to an amino acid or modified amino acid of a GIP receptor agonist peptide disclosed herein, (for example, Cys, homocystein, Lys, Orn, Dab, Dap, or p-amine-phenylalanine), represents X-L-, wherein L represents a bivalent linker comprising PEG and/or one or more amino acids or consisting of PEG and/or one or more amino acids, and X represents a substituent group. A known PEG linker, an amino acid linker or combinations thereof may be used as the bivalent linker as long as it is able to link Lys to a substituent group. Alternatively, preferably, R represents X-L-, wherein L represents a bond or a bivalent substituent group, and X represents an optionally substituted hydrocarbon group, or a salt thereof. A known bivalent substituent group including, but are not limited to, an alkylene group, a carbonyl group, an oxycarbonyl group, an imino group, an alkylimino group, a sulfonyl group, an oxy group, a sulfide group, an ester bond, an amide bond, a carbonate bond or combinations thereof may be used. More preferably, R represents X-L-, wherein L is one or a combination of more than one selected from

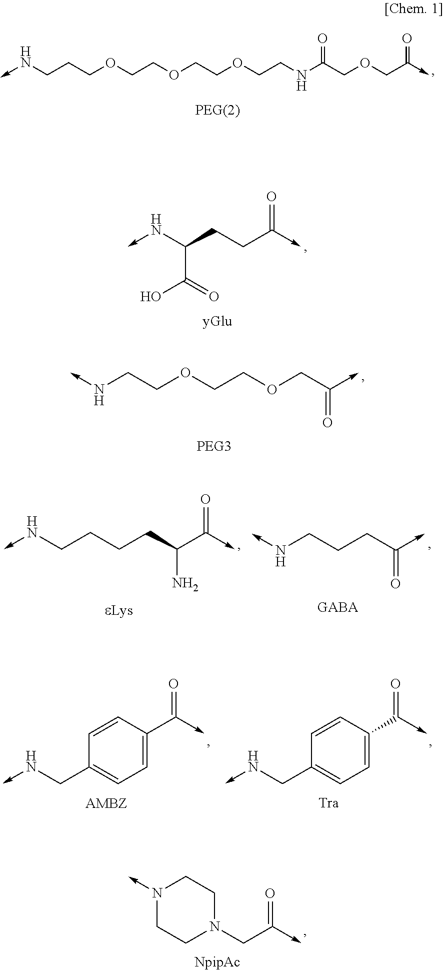

a glycine linker comprising one or two to nine-linked glycine(s) or a single bond, and X represents C₆-C₂₀ monoacid or diacid, or an acetyl group.

In various examples, R represents X-L-, wherein X-L- preferably represents Trda-GGGG-(Trda:C13 diacid), Trda-GGGGG-, Trda-GGGGGG-, Teda-GGGG-(Teda:C14 diacid), Teda-GGGGG-, Teda-GGGGGG-, Peda-GGGG-(Peda:C15 diadic), Peda-GGGGG-, Peda-GGGGGG-, Heda-GGGG-(Heda:C16 diacid), Heda-GGGGG-, Heda-GGGGGG-, Hepda-GGGG-(Hepda:C17 diacid), Hepda-GGGGG-, Hepda-GGGGGG-, Oda-GGGG-(Oda:C18 diacid), Oda-GGGGG-, Oda-GGGGGG-, Eda-GGGG-(Eda:C20 diacid), Eda-GGGGG-, Eda-GGGGGG-, Eda-GGGGGGGGG-.

Alternatively, particularly preferably, the (R) group represents X-L-, wherein L represents a glycine linker comprising five or six-linked glycines, and X represents C₈-C₂₀ linear saturated dicarboxylic acid.

Alternatively, particularly preferably, the (R) group represents X-L-, wherein L represents a bond or a bivalent substituent group, and X represents an a C8-C20 fatty acid, or a salt thereof. In some embodiments, the X represents a palmitic fatty acid used to add a palmitoyl (R) group to the epsilon amine side group of a Lysine reside in the GIP receptor agonist peptide.

Additionally, when any one or two amino acids selected from A8 to A42 optionally represent Lys(R), preferably one amino acid selected from A12, A14, A16, A17, A20, A30, A37, A40 represents Lys(R), and more preferably one amino acid selected from A16, A17, A20, A37, and A40 represents Lys(R).

In other embodiments, the GIP receptor agonist peptide has one, or two or three modified lysine residues Lys(R), wherein the (R) group represents X-L-, wherein L represents a glycine linker comprising three, four, five or six-linked glycines, and X represents C₁₆-C₂₀ linear saturated dicarboxylic acid. In an embodiment, the acyl group is a C₈ to C₂₀ fatty acyl group, for example a palmitoyl or myristoyl fatty acyl group.

In an embodiment, the GIP receptor agonist peptide is covalently attached to an (R) group, wherein the (R) group is a hydrophilic polymer at any amino acid position from A16 to the C-terminus. In an embodiment, the GIP receptor agonist peptide is covalently attached to a hydrophilic polymer at amino acid position A16, A17, A20, A30, A37, A40, A43 and A44. For example, the hydrophilic polymer may be attached to the side chain of Cys, homocystein, Lys, Orn, Dab, Dap, or p-amine-phenylalanine of the GIP receptor agonist peptide. In an embodiment, the hydrophilic polymer is a polyethylene glycol (PEG). For example, the PEG has a molecular weight of about 1,000 Daltons to about 40,000 Daltons, such as about 5,000 Daltons to about 40,000 Daltons, preferably about 1,000 Daltons, or 5,000 Daltons, or 10,000 Daltons, or 12,000 Daltons, or 14,000 Daltons to about 20,000 Daltons.

In some embodiments, methods for conjugating a polyethylene glycol (PEG) polymer to a reactive amine or sulf group is well known in the art. For example, PEG can be conjugated to a lysine amine sidechain using an amine-reactive pegylated crosslinker. A Bis(succinimidyl)penta(ethylene glycol) spacer arm can be used as a homobifunctional, amine-to-amine crosslinker that contain N-hydroxy-succinimide (NHS) esters at both ends of a PEG spacer arm. An amine-reactive crosslinker that contains a PEG spacer arm. A bis-succinimide ester-activated PEG compound may be used for crosslinking between primary amines (—NH₂) in GIP receptor agonist peptides of the present disclosure. The N-hydroxysuccinimide ester (NHS) groups at either end of the PEG spacer react specifically and efficiently with lysine and N-terminal amino groups at pH 7-9 to form stable amide bonds. Other homobifunctional, sulfhydryl-reactive crosslinkers that contain the maleimide group at either end of a PEG spacer may be used to couple PEG to a Cys amino acid of a GIP receptor agonist peptide. Heterofunctional crosslinking spacer arms may also be used when

A sulfhydryl-reactive crosslinker that contains a PEG spacer arm, may be used to couple a PEG polymer to a GIP receptor agonist peptide. In some embodiments, a bismaleimide-activated PEG compound may be used for crosslinking between sulfhydryl (—SH) groups in proteins and other thiol molecules. The maleimide groups at either end of the PEG spacer may react specifically and efficiently with reduced sulfhydryls at pH 6.5-7.5 to form stable thioether bonds. In other embodiments, direct coupling of a PEG molecule to a GIP receptor agonist peptide may be accomplished using known methods in the art. For example, a well known technique whereby a peptide may be covalently modified with PEG groups requiring PEG compounds that contain a reactive or targetable functional group at one end. The simplest method to pegylate peptides, which are rich in surface primary amines, is to use a PEG compound that contains an NHS ester group at one end, for example, a methyl-(PEG)n-NHS ester. In a similar fashion, methyl-(PEG)n-maleimide (wherein n can be from 20-300) may be used to couple a PEG molecule to a Cys containing peptide of the present disclosure. Methods known in the art for conjugation of polyethylene glycol polymers of various lengths ranging from 1,000 Daltons to 20,000 Daltons or more are provided in 1. Hermanson, G. T. (2013). 3rd Edition. Bioconjugate Techniques, Academic Press, and Veronese, F. and Harris, J. M. Eds. (2002). Peptide and protein PEGylation. Advanced Drug Delivery Review 54(4), 453-609, the disclosures of both of these references are hereby incorporated by reference in their entireties.

In some embodiments, the present disclosure provides a method for treating emesis comprising administering a therapeutically effective amount of a GIP receptor agonist peptide comprising an amino acid sequence represented by the following formula A: Tyr-A2-A3-Gly-Thr-Phe-A7-Ser-Asp-Tyr-Ser-A12-A13-A14-A15-Lys-A17-A18-A19-A20-A21-A22-A23-A24-Trp-Leu-A27-A28-A29-Y

wherein
A2 is selected from the group consisting of Ala, Gly, Sarcosine, Aib, d-Ala and d-Ser;
A3 is selected from the group consisting of Glu and Gln;
A7 is selected from the group consisting of Thr, Ile and Ser;
A12 is selected from the group consisting of Ile, Glu and Asp;
A13 is selected from the group consisting of an amino acid residue having aryl group and is selected from Tyr, Phe, Phe(4-F), Phe(4-NO2), Phe(4-NH2), Ala, Ala(2-thienyl), Ala(benzothienyl), Ala(4-Pyridyl) and phenylglycine;
A14 is selected from the group consisting of Met or oxidized Met, Leu, Val, Norleucine and Ile;
A15 is selected from the group consisting of Glu and Asp;
A17 is selected from the group consisting of Ile, Glu, and Gln;
A18 is selected from the group consisting of Ala and His;
A19 is selected from the group consisting of Val, Ala, Leu, Gln and Ile;
A20 is selected from the group consisting of Arg, Lys-Z, Gln, Glu, Asp and Cys-Z;
A21 is selected from the group consisting of Glu, Asp and Leu;
A22 is selected from the group consisting of Phe, Phe(4-F), Phe(4-Cl), Tyr, Tyr(4-Me) and Nal;
A23 is selected from the group consisting of Ile and Val;
A24 is selected from the group consisting of Ala, Asn, Glu, Lys-Z and Cys-Z;
A27 is selected from the group consisting of Val, Leu, Ala, and Lys-Z;
A28 is selected from the group consisting of Lys-Z, Ala, Arg, and Asn;
A29 is selected from the group consisting of Gly, Gln and Arg;
Y is selected from the group consisting of: A30 and -Gly-Pro-Ser-Ser-Gly-Ala-Pro-Pro-Pro-Ser-A40, or absent;
A30 and A40 are independently selected from the group consisting of -(Lys)ₙ-Z, Gly, and -Cys-Z, or absent;
Z is selected from the group consisting of -(Glu)ₘ-PEG, -(Glu)ₘ-biotin and -(Glu)ₘ-fatty acid, or absent;
n is an integer selected from 1 to 6;
m is an integer selected from 0 to 3; wherein, a lactam linkage is optionally formed between the amino acids at positions i and i+4 of the GIP receptor agonist peptide; i is an integer selected from 12 to 24.

In some embodiments, the present disclosure provides a method for treating emesis comprising administering a therapeutically effective amount of a GIP receptor agonist peptide comprising an amino acid sequence represented by the following formula B: (Y)ₙ—R₁-Ala-Glu-Gly-Thr-Phe-Ile-Ser-Asp-Tyr-Ser-Ile-Ala-Met-Asp-R₂-Ile-His-Gln-Gln-Asp-Phe-Val-Asn-Trp-Leu-Leu-Ala-Gln-R₃—NH₂

wherein: R₁ is selected from the group consisting of: (D)Tyr; DesNH₂-Tyr; (D)Phe; DesNH₂-Phe; (D)Trp; (D)₃Pya; 2-Cl-(D)Phe; 3-Cl-(D)Phe; 4-Cl-(D)Phe; 2-F-(D)Phe; 3-F-(D)Phe; 3,5-DiF(D)Phe; and 3,4,5-TriF(D)Phe;

### R₂ is Lys or Ala;

### R₃ is Lys or PEGylated Lys;

Y is acyl; n is 1 when R₁ is (D)Tyr; (D)Phe; (D)Trp; (D)₃Pya; 2-Cl-(D)Phe; 3-Cl-(D)Phe; 4-Cl-(D)Phe; 2-F-(D)Phe; 3-F-(D)Phe; 3,5-DiF(D)Phe; or 3,4,5-TriF(D)Phe; and n is 0 when R₁ is DesNH₂-Tyr or DesNH₂-Phe; or a pharmaceutically-acceptable salt thereof.

In some embodiments, the present disclosure provides a method for treating emesis comprising administering a therapeutically effective amount of a GIP receptor agonist peptide as disclosed in United States Patent Application Publication No. 2011/0136737, Ser. No. 12/377,856, filed on Aug. 17, 2007, and in particular, the GIP peptides disclosed in paragraphs [0333], [0362] and paragraphs [0412-0413], the disclosure of which is incorporated herein by reference in its entirety.

In some embodiments, the present disclosure provides a method for treating emesis comprising administering a therapeutically effective amount of a GIP receptor agonist peptide comprising an amino acid sequence represented by the following formula C: R¹-Tyr-X2-Glu-Gly-Thr-Phe-Ile-Ser-Asp-X10-X11-X12-Glu-Leu-X15-X16-X17-X18-X19-X20-X21-Phe-X23-X24-X25-Leu-X27-X28-X29-Y1-Y2-R² (I); wherein

R¹ is H—, Ac or pGlu pyroglutamic acid (pGlu; (S)-(−)-2-pyrrolidone-5-carboxylic acid),
C1-4 alkyl, acetyl, formyl, benzoyl and trifluoroacetyl,

### X₂ is Aib, Ala, D-Ala, Gly, Ser, N-Me-Ser, Ac3c, Ac4c or Ac5c;

### X10 is Tyr, Leu or Ser;

### X11 is Ser or Leu;

### X12 is Lys, ψ or Ile;

### X15 is Asp or Glu;

### X16 is Ser, Glu, Lys or ψ;

### X17 is Ile, Lys, Gin, Arg or ψ;

### X18 is His, Arg or Ala;

### X19 is Gin, Lys, Ala or Glu;

### X20 is Gin, Lys, Ala, His or Arg;

### X21 is Ala, Leu, Asp or Glu;

### X23 is Val or Ile;

### X24 is Asn or Glu;

### X25 is Tyr or Trp;

### X27 is Leu, Glu, Ser, Lys or Val;

### X28 is Ala, Ser or Arg;

X29 is Aib, Gly, Ala, Gin, Thr, Ser or Lys or is absent;
Y1 is Lys-Gly, Gly-Pro-Ser-Ser-Gly-Ala-Pro-Pro-Pro-Ser, Gly-Pro-Ser-Ser-Gly-Ala-Pro-Pro-Ser, Pro-Ser-Ser-Gly-Ala-Pro-Pro-Pro-Ser, Pro-Ser-Ser-Giy-Ala-Pro-Pro-Ser, Gly-Lys-Lys-Asn-Asp-Trp-Lys-His-Asn-Ile-Thr-Gln or absent;
Y2 is Ψ or is absent;

### R² is —NH2 or —OH;

wherein ψ is a residue independently selected from Lys, Arg, Orn and Cys and wherein the side chain of said residue is conjugated to a lipophilic substituent; and wherein the GIP analogue contains one and only one residue ψ; or a pharmaceutically acceptable salt or solvate thereof.

In some embodiments, the present disclosure provides a method for treating emesis comprising administering a therapeutically effective amount of a GIP receptor agonist peptide comprising an amino acid sequence represented by the following formula D: YX₁EGTFISDYSIX₂LDKIAQKAX₃VQWLIAGGPSSGAPPPS; wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)ₐ-CO—(CH₂)_{b}—CO₂H wherein a is 1 to 2 and b is 10 to 20; X₃ is Phe or 1-Nal; and the C-terminal amino acid is optionally amidated as a C-terminal primary amide, or a pharmaceutically acceptable salt thereof. “Aib” is alpha amino isobutyric acid, and “1-Nal” is 1-Naphthylalanine. In a further embodiment, the GIP receptor agonist peptide of formula D, comprises the amino acid sequence wherein X₁ is Aib; X₂ is Aib; K at position 20 is chemically modified through conjugation to the epsilon-amino group of the Lys side-chain with ([2-(2-Amino-ethoxy)-ethoxy]-acetyl)₂-(γGlu)ₐ-CO—(CH₂)_{b}—CO₂H wherein a is 1 to 2 and b is 10 to 18; X₃ is Phe; and the C-terminal amino acid is optionally amidated as a C-terminal primary amide, or a pharmaceutically acceptable salt thereof.

In a further embodiment, the GIP receptor agonist peptide of formula D, comprises the amino acid sequence wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)ₐ-CO—(CH₂)_{b}—CO₂H wherein a is 1 to 2 and b is 10 to 18; X₃ is 1-Nal; and the C-terminal amino acid is optionally amidated as a C-terminal primary amide, or a pharmaceutically acceptable salt thereof.

In a further embodiment, the GIP receptor agonist peptide of formula D, comprises the amino acid sequence wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)ₐ-CO—(CH₂)_{b}—CO₂H wherein a is 1 to 2 and b is 14 to 18; X₃ is Phe or 1-Nal; and the C-terminal amino acid is optionally amidated as a C-terminal primary amide, or a pharmaceutically acceptable salt thereof. In a further embodiment, the present invention provides a compound wherein b is 16 to 18. Additionally, the present invention provides a compound wherein b is 18.

In a further embodiment, the GIP receptor agonist peptide of formula D, comprises the amino acid sequence wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)ₐ-(γGlu)₁-CO—(CH₂)_{b}—CO₂H wherein a is 1 and b is 10 to 18; X₃ is Phe or 1-Nal; and the C-terminal amino acid is optionally amidated as a C-terminal primary amide, or a pharmaceutically acceptable salt thereof.

In a further embodiment, the GIP receptor agonist peptide of formula D, comprises the amino acid sequence wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)ₐ-CO—(CH₂)_{b}—CO₂H, wherein a is 2 and b is 10 to 18; X₃ is Phe or 1-Nal; and the C-terminal amino acid is optionally amidated as a C-terminal primary amide, or a pharmaceutically acceptable salt thereof.

In a further embodiment, the GIP receptor agonist peptide of formula D, comprises the amino acid sequence wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)ₐ-CO—(CH₂)_{b}—CO₂H wherein a is 1 to 2 and b is 10 to 18; X₃ is Phe or 1-Nal; and the C-terminal amino acid is amidated as a C-terminal primary amide, or a pharmaceutically acceptable salt thereof.

In a further embodiment, the GIP receptor agonist peptide of formula D, comprises the amino acid sequence wherein the amino acid sequence comprises: YX₁EGTFISDYSIX₂LDKIAQKAFVQWLIAGGPSSGAPPPS; wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)₁-CO—(CH₂)₁₈—CO₂H; and the C-terminal amino acid is amidated as a C-terminal primary amide, or a pharmaceutically acceptable salt thereof.

In a further embodiment, the GIP receptor agonist peptide of formula D, comprises the amino acid sequence wherein the amino acid sequence comprises: YX₁EGTFISDYSIX₂LDKIAQKAX₃VQWLIAGGPSSGAPPPS; wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)₂-CO—(CH₂)₁₈—CO₂H; X₃ is 1-Nal; and the C-terminal amino acid is amidated as a C-terminal primary amide, or a pharmaceutically acceptable salt thereof.

In a further embodiment, the GIP receptor agonist peptide of formula D, comprises the amino acid sequence wherein the amino acid sequence comprises: YX₁EGTFISDYSIX₂LDKIAQKAFVQWLIAGGPSSGAPPPS; wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)₁-CO—(CH₂)₁₆—CO₂H; and the C-terminal amino acid is amidated as a C-terminal primary amide, or a pharmaceutically acceptable salt thereof.

In a further embodiment, the GIP receptor agonist peptide of formula D, comprises the amino acid sequence wherein the amino acid sequence comprises: YX₁EGTFISDYSIX₂LDKIAQKAFVQWLIAGGPSSGAPPPS; wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)₂-CO—(CH₂)₁₆—CO₂H; and the C-terminal amino acid is amidated as a C-terminal primary amide, or a pharmaceutically acceptable salt thereof.

In a further embodiment, the GIP receptor agonist peptide of formula D, comprises the amino acid sequence wherein the amino acid sequence comprises: YX₁EGTFISDYSIX₂LDKIAQKAFVQWLIAGGPSSGAPPPS; wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)₂-CO—(CH₂)₁₈—CO₂H; and the C-terminal amino acid is amidated as a C-terminal primary amide, or a pharmaceutically acceptable salt thereof.

In a further embodiment, the GIP receptor agonist peptide of formula D, comprises the amino acid sequence wherein the amino acid sequence comprises: YX₁EGTFISDYSIX₂LDKIAQKAX₃VQWLIAGGPSSGAPPPS; wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)₁-CO—(CH₂)₁₆—CO₂H; X₃ is 1-Nal; and the C-terminal amino acid is amidated as a C-terminal primary amide, or a pharmaceutically acceptable salt thereof.

In a further embodiment, the GIP receptor agonist peptide of formula D, comprises the amino acid sequence wherein the amino acid sequence comprises: YX₁EGTFISDYSIX₂LDKIAQKAX₃VQWLIAGGPSSGAPPPS; wherein X₁ is Aib; X₂ is Aib; Lys at position 20 is chemically modified through conjugation to the epsilon-amino group of the Lys side-chain with ([2-(2-Amino-ethoxy)-ethoxy]-acetyl)₂-(γGlu)₂-CO—(CH₂)₁₆—CO₂H; X₃ is 1-Nal; and the C-terminal amino acid is amidated as a C-terminal primary amide, or a pharmaceutically acceptable salt thereof.

In some embodiments, the present disclosure provides a method for treating emesis comprising administering a therapeutically effective amount of a GIP receptor agonist peptide comprising an amino acid sequence represented by the following formula F: (R²R³)-Tyr-Ala-Glu-A4-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-R1, (F)

wherein:

### A⁴ is Gly, Acc, Aib, or β-Ala;

### A⁵ is Thr, Acc, Aib, or Ser;

A⁶ is Phe, Acc, Aib, Aic, Cha, 1Nal, 2Nal, 2-Pal, 3-Pal, 4-Pal, (X⁴, X⁵, X⁶, X⁷, X⁸)Phe or Trp;

### A⁷ is Ile, Abu, Acc, Aib, Ala, Cha, Leu, Nle, Phe, or Val;

### A⁸ is Ser, Aib, or Thr;

### A⁹ is Asp, Aib, or Glu;

A¹⁰ is Tyr, Acc, Cha, 1Nal, 2Nal, 2-Pal, 3-Pal, 4-Pal, Phe, or (X⁴, X⁵, X⁶, X⁷, X⁸)Phe;

### A¹¹ is Ser, Acc, Aib, Nle or Thr;

### A¹² is Ile, Abu, Acc, Aib, Ala, Cha, Leu, Nle, Phe, Tle, or Val;

A¹³ is Ala, Acc, Aib, .beta.-Ala, D-Ala, Gly, or Ser;

### A¹⁴ is Met, Abu, Acc, Aib, Ala, Cha, Ile, Leu, Nle, Phe, Tle, or Val;

### A¹⁵ is Asp, Aib, or Glu;

A¹⁶ is Lys, Amp, Apc, Arg, hArg, Orn, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃—), Pen(succinimide-N—(CH₂)ₓ—C(O)—NH—(CH₂)_{y}—CH₃—), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), or Pen(succinimide-N—(CH₂)ₛ—NH—C(O)—(CH₂)ₜ—CH₃);

### A¹⁷ is Ile, Abu, Acc, Aib, Ala, Cha, Leu, Nle, Phe, Tle, or Val;

### A¹⁸ is His, Amp, Arg, 2-Pal, 3-Pal, or 4-Pal, Phe, or Tyr;

### A¹⁹ is Gln, Aib, or Asn;

### A²⁹ is Gln, Aib, or Asn;

### A²¹ is Asp, Aib, or Glu;

A²² is Phe, Acc, Aib, Aic, Cha, 1Nal, 2Nal, 2-Pal, 3-Pal, 4-Pal, (X⁴, X⁵, X⁶, X⁷, X⁸)Phe, or Trp;

### A²³ is Val, Abu, Acc, Aib, Ala, Cha, Ile, Leu, Nle, or Tle;

### A²⁴ is Asn, Aib, or Gln;

A²⁵ is Trp, Acc, Aib, 1Nal, 2Nal, 2-Pal, 3-Pal, 4-Pal, Phe, or (X⁴, X⁵, X⁶, X⁷, X⁸)Phe;
A²⁶ is Leu, Acc, Aib, Cha, Ile, Nle, Phe, (X⁴, X⁵, X⁶, X⁷, X⁸)Phe or Tle;
A²⁷ is Leu, Acc, Aib, Cha, Ile, Nle, Phe, (X⁴, X⁵, X⁶, X⁷, X⁸)Phe or Tle;

### A²⁸ is Ala, Aib, or Acc;

A²⁹ is Gln, Aib, Asn, or absent;
A³⁰ is Lys, Amp, Apc, Arg, hArg, Orn, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)t-CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A31 is Gly, Acc, Aib, .beta.-Ala, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)_{z}—C(O)—NH—(CH₂)_{y}—CH3), Pen(succinimide-N—(CH₂)ₓ—C(O)—NH—(CH₂)_{y}—CH3), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), His, Pen(succinimide-N—(CH₂)ₛ—NH—C(O)—(CH₂)ₜ—CH₃—), or absent;
A³² is Lys, Amp, Apc, Arg, hArg, Cys, Orn, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A³³ is Lys, Amp, Apc, Arg, hArg, Cys, Orn, HN—CH((CH₂)n-N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A³⁴ is Asn, Aib, Gln, Ser, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)t-CH₃), Pen(succinimide-N—(CH₂)ₛ—NH—C(O)—(CH₂)ₜ—CH₃—), or absent;
A³⁵ is Asp, Aib, Glu, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A³⁶ is Trp, Acc, Aib, 1Nal, 2Nal, 2-Pal, 3-Pal, 4-Pal, Phe, (X⁴, X⁵, X⁶, X⁷, X⁸)Phe, HN—CH(((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A³⁷ is Lys, Amp, Apc, Arg, hArg, Orn, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A³⁸ is His, Amp, 2-Pal, 3-Pal, 4-Pal, Phe, Tyr, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A³⁹ is Asn, Aib, Gln, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)t-CH₃—), or absent;
A⁴⁰ is Ile, Acc, Aib, Ser, Thr, HN—CH((CH₂)n-N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A⁴¹ is Thr, Aib, Acc, Asn, Gln, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A⁴² is Gln, Acc, Aib, Asn, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)ₛ—NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent;
A⁴³ is Acc, Ado, Aib, Ala, Asn, Asp, Cys, Gln, His, Phe, Thr, Trp, HN—CH((CH₂)ₙ—N(R⁴R⁵))—C(O), Cys(succinimide-N-alkyl), hCys(succinimide-N-alkyl), Pen(succinimide-N-alkyl), Cys(succinimide-N—(CH₂)ₓ—C(O)—NH—(CH₂)_{y}—CH₃—), hCys(succinimide-N—(CH₂)ₓ—C(O)—NH—(CH₂)_{y}—CH₃), Pen(succinimide-N—(CH₂)ₓ—C(O)—NH—(CH₂)_{y}—CH₃), Cys(succinimide-N—(CH₂)s-NH—C(O)—(CH₂)ₜ—CH₃), hCys(succinimide-N—(CH₂)ₛ—NH—C(O)—(CH₂)ₜ—CH₃), Pen(succinimide-N—(CH₂)ₛ—NH—C(O)—(CH₂)ₜ—CH₃—), or absent; wherein R¹ is OH, NH₂, (C₁-C₃₀)alkoxy, or NH—X²—CH₂—Z⁰, wherein X² is a (C₀-C₃₀) hydrocarbon moiety, and Z⁰ is H, OH, CO₂H, or CONH₂; each of R², R³, R⁴ and R⁵ is independently selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl; provided that when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂-C₃₀)alkali, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl; further provided that when R⁴ is (C₁-C₃₀)acyl, aryl(C₁-C₃₀)acyl, substituted (C₁-C₃₀)acyl, or substituted aryl(C₁-C₃₀)acyl, then R⁵ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;
n is, independently for each occurrence, an integer from 1 to 5 inclusive;
s, t, x and y each is, independently for each occurrence, an integer from 1 to 30 inclusive; and
X⁴, X⁵, X⁶, X⁷ and X⁸ each is, independently for each occurrence, H, F, Cl, Br, I, (C₁₋₁₀)alkyl, substituted (C₁₋₁₀)alkyl, aryl, substituted aryl, OH, NH₂, NO₂, or CN.

### B. SYNTHESIS GIP RECEPTOR AGONIST PEPTIDES

The GIP receptor agonist peptide may be synthesized recombinantly or can be produced according to a peptide synthesis method known in the art. The peptide synthesis method may be any of, for example, a solid phase synthesis process and a liquid phase synthesis process. That is, the object peptide can be produced by repeating condensation of a partial peptide or amino acid capable of constituting the GIP receptor agonist peptide, and the remaining portion (which may be constituted by two or more amino acids) according to a desired sequence. When a product having the desirable sequence has a protecting group, the object GIP receptor agonist peptide can be produced by eliminating a protecting group. Examples of the condensing method and eliminating method of a protecting group to be known include methods described in the following (1)-(5).
(1) M. Bodanszky and M. A. Ondetti: Peptide Synthesis, Interscience Publishers, New York (1966)
(2) Schroeder and Luebke: The Peptide, Academic Press, New York (1965)
(3) Nobuo Izumiya, et al.: Peptide Gosei-no-Kiso to Jikken (Basics and experiments of peptide synthesis), published by Maruzen Co. (1975)
(4) Haruaki Yajima and Shunpei Sakakibara: Seikagaku Jikken Koza (Biochemical Experiment) 1, Tanpakushitsu no Kagaku (Chemistry of Proteins) IV, 205 (1977)
(5) Haruaki Yajima, ed.: Zoku Iyakuhin no Kaihatsu (A sequel to Development of Pharmaceuticals), Vol. 14, Peptide Synthesis, published by Hirokawa Shoten.

After the reaction, GIP receptor agonist peptide can be purified and isolated using conventional methods of purification, such as solvent extraction, distillation, column chromatography, liquid chromatography, recrystallization, etc., in combination thereof When the peptide obtained by the above-mentioned method is in a free form, it can be converted to a suitable salt by a known method; conversely, when the peptide is obtained in the form of a salt, the salt can be converted to a free form or other salt by a known method.

The starting compound may also be a salt. Examples of such salt include those exemplified as salts of the exemplified selective GIP receptor agonists mentioned bellow.

For condensation of protected amino acid or peptide, various activation reagents usable for peptide synthesis can be used, which are particularly preferably trisphosphonium salts, tetramethyluronium salts, carbodiimides and the like. Examples of the trisphosphonium salt include benzotriazol-1-yloxytris(pyrrolizino)phosphoniumhexafluorophosphate (PyBOP), bromotris(pyrrolizino)phosphoniumhexafluorophosphate (PyBroP), 7-azabenzotriazol-1-yloxytris(pyrrolizino)phosphoniumhexafluorophosphate (PyAOP), examples of the tetramethyluronium salt include 2-(1H-benzotriazol-1-yl)-1,1,3,3-tetramethyluroniumhexafluorophosphate (HBTU), 2-(7-azabenzotriazol-1-yl)-1,1,3,3-tetramethyluroniumhexafluorophosphate (HATU), 2-(1H-benzotriazol-1-yl)-1,1,3,3-tetramethyluroniumtetrafluoroborate (TBTU), 2-(5-norbornane-2,3-dicarboxyimide)-1,1,3,3-tetramethyluroniumtetrafluoroborate (TNTU), O—(N-succimidyl)-1,1,3,3-tetramethyluroniumtetrafluoroborate (TSTU), and examples of the carbodiimide include N,N′-Dicyclohexylcarbodiimide (DCC), N,N′-diisopropylcarbodiimide (DIPCDI), N-ethyl-N′-(3-dimethylaminopropyl)carbodiimide hydrochloride (EDCI.HCl) and the like. For condensation using these, addition of a racemization inhibitor [e.g., N-hydroxy-5-norbornene-2,3-dicarboxylic imide (HONB), 1-hydroxybenzotriazole (HOBt), 1-Hydroxy-7-azabenzotriazole (HOAt), 3,4-Dihydro-3-hydroxy-4-oxo-1,2,3-benzotriazine (HOOBt), ethyl 2-cyano-2-(hydroxyimino)acetate (Oxyma)etc.] is preferable. A solvent to be used for the condensation can be appropriately selected from those known to be usable for peptide condensation reaction. For example, acid amides such as anhydrous or water-containing N,N-dimethylformamide, N,N-dimethylacetamide, N-methylpyrrolidone and the like, halogenated hydrocarbons such as methylene chloride, chloroform and the like, alcohols such as trifluoroethanol, phenol and the like, sulfoxides such as dimethylsulfoxide and the like, tertiary amines such as pyridine and the like, ethers such as dioxane, tetrahydrofuran and the like, nitriles such as acetonitrile, propionitrile and the like, esters such as methyl acetate, ethyl acetate and the like, an appropriate mixture of these and the like can be used. Reaction temperature is appropriately selected from the range known to be usable for peptide binding reactions, and is normally selected from the range of about −20° C. to 90° C. An activated amino acid derivative is normally used from 1.5 to 6 times in excess. In solid phase synthesis, when a test using the ninhydrin reaction reveals that the condensation is insufficient, sufficient condensation can be conducted by repeating the condensation reaction without elimination of protecting groups. If the condensation is yet insufficient even after repeating the reaction, unreacted amino acids can be acylated with acetic anhydride, acetylimidazole or the like so that an influence on the subsequent reactions can be avoided.

Examples of the protecting groups for the amino groups of the starting amino acid include benzyloxycarbonyl (Z), tert-butoxycarbonyl (Boc), tert-pentyloxycarbonyl, isobornyloxycarbonyl, 4-methoxybenzyloxycarbonyl, 2-chlorobenzyloxycarbonyl (Cl—Z), 2-bromobenzyloxycarbonyl (Br—Z), adamantyloxycarbonyl, trifluoroacetyl, phthaloyl, formyl, 2-nitrophenylsulphenyl, diphenylphosphinothioyl, 9-fluorenylmethyloxycarbonyl (Fmoc), trityl and the like.

Examples of the carboxyl-protecting group for the starting amino acid include aryl, 2-adamantyl, 4-nitrobenzyl, 4-methoxybenzyl, 4-chlorobenzyl, phenacyl and benzyloxycarbonylhydrazide, tert-butoxycarbonylhydrazide, tritylhydrazide and the like, in addition to the above-mentioned C₁₋₆ alkyl group, C₃₋₁₀ cycloalkyl group, C₇₋₁₄ aralkyl group.

The hydroxyl group of serine or threonine can be protected, for example, by esterification or etherification. Examples of the group suitable for the esterification include lower (C₂₋₄) alkanoyl groups such as an acetyl group and the like, aroyl groups such as a benzoyl group and the like, and a group derived from an organic acid and the like. In addition, examples of the group suitable for etherification include benzyl, tetrahydropyranyl, tert-butyl(Bu^{t}), trityl (Trt) and the like.

Examples of the protecting group for the phenolic hydroxyl group of tyrosine include Bzl, 2,6-dichlorobenzyl, 2-nitrobenzyl, Br—Z, tert-butyl and the like.

Examples of the protecting group for the imidazole of histidine include p-toluenesulfonyl (Tos), 4-methoxy-2,3,6-trimethylbenzenesulfonyl (Mtr), dinitrophenyl (DNP), benzyloxymethyl (Bom), tert-butoxymethyl (Bum), Boc, Trt, Fmoc and the like.

Examples of the protecting group for the guanidino group of arginine include Tos, Z, 4-methoxy-2,3,6-trimethylbenzenesulfonyl (Mtr), p-methoxybenzenesulfonyl (MBS), 2,2,5,7,8-pentamethylchromane-6-sulfonyl (Pmc), mesitylene-2-sulfonyl (Mts), 2,2,4,6,7-pentamethyldihydrobenzofuran-5-sulfonyl (Pbf), Boc, Z, NO2 and the like.

Examples of the protecting group for a side chain amino group of lysine include Z, Cl—Z, trifluoroacetyl, Boc, Fmoc, Trt, Mtr, 4,4-dimethyl-2,6-dioxocyclohexylideneyl (Dde) and the like.

Examples of the protecting group for indolyl of tryptophan include formyl (For), Z, Boc, Mts, Mtr and the like.

Examples of the protecting group for asparagine and glutamine include Trt, xanthyl (Xan), 4,4′-dimethoxybenzhydryl (Mbh), 2,4,6-trimethoxybenzyl (Tmob) and the like.

Examples of activated carboxyl groups in the starting material include corresponding acid anhydride, azide, active esters [ester with alcohol (e.g., pentachlorophenol, 2,4,5-trichlorophenol, 2,4-dinitrophenol, cyanomethylalcohol, paranitrophenol, HONB, N-hydroxysuccimide, 1-hydroxybenzotriazole (HOBt), 1-hydroxy-7-azabenzotriazole(HOAt))] and the like. Examples of the activated amino group in the starting material include corresponding phosphorous amide.

Examples of the method for removing (eliminating) a protecting group include a catalytic reduction in a hydrogen stream in the presence of a catalyst such as Pd-black or Pd-carbon; an acid treatment using anhydrous hydrogen fluoride, methanesulfonic acid, trifluoromethanesulfonic acid, trifluoroacetic acid (TFA), trimethylsilyl bromide (TMSBr), trimethylsilyl trifluoromethanesulfonate, tetrafluoroboric acid, tris(trifluoro)boric acid, boron tribromide, or a mixture solution thereof; a base treatment using diisopropylethylamine, triethylamine, piperidine, piperazine or the like; and reduction with sodium in liquid ammonia, and the like. The elimination reaction by the above-described acid treatment is generally carried out at a temperature of −20° C. to 40° C.; the acid treatment is efficiently conducted by adding a cation scavenger such as anisole, phenol, thioanisole, metacresol and paracresol; dimethylsulfide, 1,4-butanedithiol, 1,2-ethanedithiol, triisopropylsilane and the like. Also, a 2,4-dinitrophenyl group used as a protecting group of the imidazole of histidine is removed by thiophenol treatment; a formyl group used as a protecting group of the indole of tryptophan is removed by deprotection by acid treatment in the presence of 1,2-ethanedithiol, 1,4-butanedithiol, or the like, as well as by alkali treatment with dilute sodium hydroxide, dilute ammonia, or the like.

Protection of a functional group that should not be involved in the reaction of a starting material and a protecting group, elimination of the protecting group, activation of a functional group involved in the reaction and the like can be appropriately selected from known protecting groups and known means.

In a method of preparing an amide of the peptide, it is formed by a solid phase synthesis using a resin for amide synthesis, or the α-carboxyl group of the carboxy terminal amino acid is amidated, and a peptide chain is elongated to a desired chain length toward the amino group side, thereafter a peptide wherein the protecting group for the N-terminal α-amino group of the peptide chain only removed and a peptide wherein the protecting group for the C-terminal carboxyl group only removed of the peptide chain are prepared, and the both peptides are condensed in a mixed solvent described above. For details about the condensation reaction, the same as above applies. After the protected peptide obtained by the condensation is purified, all protecting groups can be removed by the above-described method to yield a desired crude polypeptide. By purifying this crude peptide using various publicly known means of purification, and freeze-drying the main fraction, a desired amide of the peptide can be prepared.

When the GIP receptor agonist peptide is present as a configurational isomer such as enantiomer, diastereomer etc., a conformer or the like, they are also encompassed within the description of a GIP receptor agonist peptide and each can be isolated by a means known per se or the above separation and purification methods on demand. In addition, when the GIP receptor agonist peptide is in the form of a racemate, it can be separated into S- and R-forms by conventional optical resolution.

When a GIP receptor agonist peptide includes stereoisomers, both the isomers alone and mixtures of each isomers are also encompassed within the meaning of a GIP receptor agonist peptide. A GIP receptor agonist peptide can be chemically modified according to a method known per se and using substituent and polyethylene glycol. For example, a chemically modified GIP receptor agonist peptide can be produced by introducing substituent and/or conjugatedly binding polyethylene glycol to Cys residue, Asp residue, Glu residue, Lys residue and the like of a GIP receptor agonist peptide. Additionally, there may be a linker structure between the amino acid of the GIP receptor agonist peptide and substituent and polyethylene glycol.

A GIP receptor agonist peptide modified by a substituent and/or polyethylene glycol (PEG) produces for example, one or more effects related to promoting the biological activity, prolonging the blood circulation time, reducing the immunogenicity, enhancing the solubility, and enhancing the resistance to metabolism, of a therapeutically and diagnostically important peptide.

The molecular weight of PEG is not particularly limited and is normally about 1 K to about 1000 K daltons, preferably about 10 K to about 100 K daltons, more preferably about 20 K to about 60 K daltons.

Modifying a GIP receptor agonist peptide by addition or coupling a substituent can be conducted by introducing the substituent based on known oxidation reaction and reduction reaction.

A method well known in the art can be used as a method for modifying a GIP receptor agonist peptide by PEG, and, for example, the methods described below can be used. (1) A PEGylating reagent having an active ester (e.g., SUNBRIGHT MEGC-30TS (trade name), NOF Corp.) is bound to an amino group of the GIP receptor agonist peptide. (2) A PEGylating reagent having an aldehyde (e.g., SUNBRIGHT ME-300AL (trade name), NOF Corp.) is bound to the amino group of the GIP receptor agonist peptide. (3) A divalent cross-linking reagent (e.g., GMBS (Dojindo Laboratories), EMCS (Dojindo Laboratories), KMUS (Dojindo Laboratories), SMCC (Pierce)) is bound to an amino acid, (for example, a Lys and/or a Cys), of the GIP receptor agonist peptide, to which a PEGylating reagent having a thiol group (e.g., SUNBRIGHT ME-300-SH (trade name), NOF Corp.) is then bound. (4) A thiol group is introduced to a GIP receptor agonist peptide through an SH-introducing agent (e.g., D-cysteine residue, L-cysteine residue, Traut's reagent), and this thiol group is reacted with a PEGylating reagent having a maleimide group (e.g., SUNBRIGHT ME-300MA (trade name), NOF Corp.). (5) A thiol group is introduced to GIP receptor agonist peptide through an SH-introducing agent (e.g., D-cysteine residue, L-cysteine residue, Traut's reagent), and this thiol group is reacted with a PEGylating reagent having an iodoacetamide group (e.g., SUNBRIGHT ME-300IA (trade name), NOF Corp.). (6) A ω-aminocarboxylic acid, an α-amino acid or the like is introduced as a linker to the N-terminal amino group of a GIP receptor agonist peptide, and an amino group derived from this linker is reacted with a PEGylating reagent having an active ester (e.g., SUNBRIGHT MEGC-30TS (trade name), NOF Corp.). (7) A co-aminocarboxylic acid, an α-amino acid or the like is introduced as a linker to the N-terminal amino group of a GIP receptor agonist peptide, and an amino group derived from this linker is reacted with a PEGylating reagent having an aldehyde group (e.g., SUNBRIGHT ME-300AL (trade name), NOF Corp.).

In addition, the GIP receptor agonist peptide may be a solvate (e.g., hydrate) or a non-solvate (e.g., non-hydrate).

The GIP receptor agonist peptide may be labeled with an isotope (e.g., ³H, ¹⁴C, ³⁵S, ¹²⁵I) or the like.

Furthermore, GIP receptor agonist peptide may be a deuterium conversion form wherein ¹H is converted to ²H(D).

In some embodiments, a GIP receptor agonist peptide labeled with or substituted with an isotope can be used as, for example, a tracer (PET tracer) for use in Positron Emission Tomography (PET), and is useful in the fields of medical diagnosis and the like.

For the GIP receptor agonist peptide mentioned herein, the left end is the N-terminal (amino terminal) and the right end is the C-terminal (carboxyl terminal) in accordance with the conventional peptide marking. The C-terminal of peptide may be any of an amide (—CONH₂), a carboxyl group (—COOH), a carboxylate (—COO⁻), an alkylamide (—CONHR^{a}), and an ester (—COOR^{a}). Particularly, amide (—CONH₂) is preferable.

A GIP receptor agonist peptide of the present disclosure may be in a salt form. Examples of such salt include metal salts, ammonium salts, salts with organic base, salts with inorganic acid, salts with organic acid, salts with basic or acidic amino acid, and the like.

Preferable examples of the metal salt include alkali metal salts such as sodium salt, potassium salt and the like; alkaline earth metal salts such as calcium salt, magnesium salt, barium salt and the like; aluminum salt and the like.

Preferable examples of the salt with organic base include salts with trimethylamine, triethylamine, pyridine, picoline, 2,6-lutidine, ethanolamine, diethanolamine, triethanolamine, cyclohexylamine, dicyclohexylamine, N,N-dibenzylethylenediamine and the like.

Preferable examples of the salt with inorganic acid include salts with hydrochloric acid, hydrobromic acid, nitric acid, sulfuric acid, phosphoric acid and the like.

Preferable examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include salts with arginine, lysine, ornithine and the like. Preferable examples of the salt with acidic amino acid include salts with aspartic acid, glutamic acid and the like.

Among the above-mentioned salts, a pharmaceutically acceptable salt is preferable. For example, when a compound has an acidic functional group, an inorganic salt such as alkali metal salt (e.g., sodium salt, potassium salt etc.), alkaline earth metal salt (e.g., calcium salt, magnesium salt, barium salt etc.) and the like, ammonium salt etc., and when a compound has a basic functional group, for example, a salt with inorganic acid such as hydrochloric acid, hydrobromic acid, nitric acid, sulfuric acid, phosphoric acid and the like, or a salt with organic acid such as acetic acid, phthalic acid, fumaric acid, oxalic acid, tartaric acid, maleic acid, citric acid, succinic acid, methanesulfonic acid, p-toluenesulfonic acid and the like are preferable.

In some embodiments, the GIP receptor agonist peptide may be synthesized and/or used in a prodrug form to treat or prevent emesis. A prodrug means a compound which is converted to a GIP receptor agonist peptide with a reaction due to an enzyme, gastric acid, etc. under the physiological condition in the living body, that is, a compound which is converted to a GIP receptor agonist peptide with oxidation, reduction, hydrolysis, etc. according to an enzyme; a polypeptide which is converted to GIP receptor agonist peptide by hydrolysis etc. due to gastric acid, etc.

Examples of a prodrug of a GIP receptor agonist peptide may include a compound wherein an amino group of a GIP receptor agonist peptide is acylated, alkylated or phosphorylated (e.g., compound wherein amino group of a GIP receptor agonist peptide is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, and the like); a compound wherein a hydroxy group of a GIP receptor agonist peptide is acylated, alkylated, phosphorylated or borated (e.g., a compound wherein a hydroxy group of a GIP receptor agonist peptide is acetylated, palmitoylated, propanoylated, pivaloylated, succinylated, fumarylated, alanylated or dimethylaminomethylcarbonylated); a compound wherein a carboxy group of a GIP receptor agonist peptide is esterified or amidated (e.g., a compound wherein a carboxy group of a GIP receptor agonist peptide is C₁₋₆ alkyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated) and the like. Among others, a compound wherein a carboxy group of a GIP receptor agonist peptide is esterified with C₁₋₆ alkyl such as methyl, ethyl, tert-butyl or the like is preferably used. These compounds, peptides and polypeptides can be produced from a GIP receptor agonist peptide by a method known per se.

A prodrug of a GIP receptor agonist peptide may also be one which is converted into a GIP receptor agonist peptide under a physiological condition, such as those described in IYAKUHIN no KAIHATSU (Development of Pharmaceuticals), Vol. 7, Design of Molecules, p. 163-198, Published by HIROKAWA SHOTEN (1990).

In the present specification, the prodrug may form a salt. Examples of such a salt include those exemplified as the salt of a GIP receptor agonist peptide.

In some embodiments, a GIP receptor agonist peptide may be synthesized and/or used as a crystal. Crystals having a singular crystal form or a mixture of plural crystal forms are also encompassed by the examples of GIP receptor agonist peptides. Crystals can be produced by crystallizing a GIP receptor agonist peptide according to a crystallization method known per se.

In addition, a GIP receptor agonist peptide may be a pharmaceutically acceptable cocrystal or cocrystal salt. Here, the cocrystal or cocrystal salt means a crystalline substance consisting of two or more particular substances which are solids at room temperature, each having different physical properties (e.g., structure, melting point, heat of melting, hygroscopicity, solubility, stability etc.). The cocrystal and cocrystal salt can be produced by cocrystallization known per se.

The crystal of a GIP receptor agonist peptide is superior in physicochemical properties (e.g., melting point, solubility, stability) and biological properties (e.g., pharmacokinetics (absorption, distribution, metabolism, excretion), efficacy expression), and thus it is extremely useful as a medicament.

In some embodiments, a GIP receptor agonist peptide and/or a prodrug thereof (hereinafter to be sometimes abbreviated as a GIP receptor agonist peptide of the present disclosure) have a GIP receptor activating action, and may have selectivity as agonists of the GIP receptor over other receptors such as the GLP1R. The compounds of the present disclosure have a high GIP receptor selective activation action in vivo.

### C. METHODS OF PROPHYLAXIS AND TREATMENT OF EMESIS

GIP is gastric inhibitory polypeptide or glucose-dependent insulinotropic peptide is an incretin hormone and has a promoting action on insulin secretion from the pancreas. Incretins are closely related to glucose metabolism and thus the compound having a GIP receptor activation action is useful for preventing and treating symptoms related to abnormal glucose metabolism including diabetes and obesity. Additionally, the compounds of the present disclosure have a GIP receptor selective activation action and suppress vomiting by activating GABAergic neurons in the area postrema.

More specifically, the GIP receptor agonist peptides of the present disclosure have a an antiemetic action, and may inhibit the occurrence of nausea, and/or vomiting when associated with various stimuli disclosed herein, for example, when a subject has cyclic vomiting syndrome or is administered a chemotherapeutic drug, for example, a chemotherapeutic drug with emetic potential, such as platinum based chemotherapeutics such as cisplatin, oxaliplatin, and carboplatin; irinotecan and other topo isomerase inhibitors used in the treatment of cancer. The GIP receptor agonist peptides of the present disclosure have a high chemical stability and excellent persistence of the effects in vivo.

The GIP receptor agonist peptides of the present disclosure may be used as a GIP receptor activator. In the present disclosure, the GIP receptor activator (GIP receptor agonist) means an agent having a GIP receptor activation action. Additionally, the GIP receptor selective activator (GIP receptor selective agonist) specifically means an agent having an EC₅₀ for the GIP receptor of 1/1000 or less, and preferably 1/10000 or less, times the EC50 for the GLP-1 receptor, or in other words the ratio of EC₅₀ GLP1R/EC₅₀ GIPR is greater than 10, greater than 100 or greater than 1,000, or greater than 10,000.

The GIP receptor agonist peptides of the present disclosure have low toxicity (e.g., acute toxicity, chronic toxicity, genetic toxicity, reproductive toxicity, cardiac toxicity, carcinogenicity), shows a few side effects, and can be safely administered to a mammal (e.g., human, bovine, horse, dog, cat, monkey, mouse, rat) as an agent for the prophylaxis or treatment of emesis.

“Treatment,” in the context of treating emesis by administering at least one of the GIP receptor agonist peptides disclosed herein, includes both prophylactic treatment and the treatment of emesis after a subject experiences emesis. Prophylactic treatment includes administration of a GIP receptor agonist peptide before a subject experiences emesis, such as when the subject experiences nausea, as well as administration of the GIP receptor agonist peptide before the subject is exposed to a substance, agent, or event, or before the subject contracts a condition, which results in or is likely to result in the subject experiencing emesis. As used herein, “therapeutically effective amount” refers to an amount sufficient to elicit the desired biological response. In the present invention, the desired biological response is treating or preventing emesis.

The GIP receptor agonist peptides of the present disclosure can be used to treat or prevent emesis in a subject, for example, a mammal, for example, humans, non-human primates, apes, monkeys, laboratory mammals for example, mice, rats, rabbits, guinea-pigs, ferrets, domesticated mammals, such as companion mammals, dogs, cats and horses, and farm mammals, such as cattle, pigs, sheep and goats purely as examples, but not intended to be an exhaustive list. In each of these cases, the methods of the present disclosure are provided to treat or prevent emesis in a subject in need thereof, to reduce or inhibit emesis, to reduce or inhibit a symptom associated with emesis, or to reduce or inhibit nausea and/or vomiting.

In order to prevent or treat emesis, an effective amount of one or more of the present compounds in a pharmaceutical composition is administered to a subject/patient (used interchangeably herein) in need thereof. A subject is determined to be in need of treatment with the present GIP receptor agonist peptide either through observation of vomiting by the subject, or through a subject's self-reporting of emesis (in the case of a human subject). A patient is determined to be in need of preventative therapy by assessing that the patient is at risk of experiencing emesis due to another medical condition or due to exposure to an agent known to be associated with emesis, such as an infection by a virus or bacteria or chemical agent or radiation.

The GIP receptor agonist peptides of the present disclosure can be used to treat or prevent emesis in a subject for a variety of emetogenic, causes, conditions, treatments or stimuli, for example, (1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome, chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness); (2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;

(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium; and
(6) Vomiting and nausea caused by a vestibular disorder such as motion sickness or dizziness
(7) Opioid withdrawal;
(8) Pregnancy including hyperemesis gravidarium;
(9) A vestibular disorder such as motion sickness or dizziness; and
(10) A physical injury causing local, systemic, acute or chronic pain.

The present GIP receptor agonist peptides are beneficial in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, pain, opioid use and withdrawal, migraine, and variations in intracranial pressure. The uses of this invention are of particular benefit in the therapy of emesis induced by radiation, for example during the treatment of cancer, or radiation sickness, and in the treatment of post-operative nausea and vomiting. Most especially, use of the invention is beneficial in the therapy of emesis induced by antineoplastic (cytotoxic) agents including those routinely used in cancer chemotherapy, emesis induced by other pharmacological agents, for example, alpha-2 adrenoceptor antagonists, such as yohimbine, MK-912 and MK-467, and type IV cyclic nucleotide phosphodiesterase (PDE4) inhibitors, such as RS14203, CT-2450 and rolipram.

Particular examples of chemotherapeutic agents are described, for example, by D. J. Stewart in Nausea and Vomiting: Recent Research and Clinical Advances, ed. J. Kucharczyk et al., CRC Press Inc., Boca Raton, Fla., USA, 1991, pages 177-203, especially page 188. Commonly used chemotherapeutic agents include cisplatin, carboplatin, oxaliplatin, cyclophosphamide, dacarbazine (DTIC), dactinomycin, mechlorethamine (nitrogen mustard), streptozocin, cyclophosphamide, carmustine (BCNU), irinotecan, and other topoisomerase inhibitors, lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil (R. J. Gralle et al. in Cancer Treatment Reports, 1984, 68, 163-172). Emesis due to other chemical agents, such as the toxins soman or sarin, or opioid drug usage and/or withdrawal, e.g. morphine, heroin, oxycodone, and the like can also be prevented and/or treated.

The present compounds are administered to a patient in a quantity sufficient to treat or prevent the symptoms and/or underlying etiology associated with emesis in the patient. In a preferred embodiment, the GIP receptor agonist peptides are administered prior to administration of an agent which is likely to cause emesis, such as one or more of the chemotherapeutic agents described above. The present GIP receptor agonist peptides can also be administered in combination with such agents, either in physical combination or in combined therapy through the administration of the present compounds and agents in succession (in any order). Although the present invention is useful in any mammal suffering from emesis, a preferred subject is a human.

In some embodiments, the selective GIP receptor agonists of the present disclosure may be administered to treat emesis when a subject is concomitantly being treated for diabetes and/or obesity. Several known anti-diabetic medicaments are known for causing emesis, for example, Metformin (Glucophage, Glumetza, others), sulfonylureas, meglitinides, thiazolidinediones, DPP-4 inhibitors, SGLT2 inhibitors, and GLP-1 receptor agonists. In some embodiments, methods for treating emesis in a subject, for example in a subject in need thereof, may include administering an effective amount of a GIP receptor agonist peptide to a subject that does not have type-2 diabetes mellitus or a subject that is not taking a medicament to treat type-2 diabetes mellitus while experiencing emesis.

Nausea is a subjective unpleasant feeling in the back of one's throat and stomach that may lead to vomiting. There are many words that describe nausea including, but not limited to: sick to my stomach, queasy, or upset stomach. Nausea can have other symptoms that happen at the same time, such as increased saliva (spit), dizziness, light-headedness, trouble swallowing, skin temperature changes, and a fast heart rate. Vomiting is also described as “throwing up.” When one vomits, one's abdominal muscles contract (squeeze) and push the contents of one's stomach out through their mouth. One might or might not feel nauseated. Retching is when one tries to vomit without bringing anything up from one's stomach. Other words used to describe retching are gagging or dry heaves. Nausea and vomiting often happen at the same time, but they can be two different conditions that may be mutually exclusive or mutually associated. Some chemotherapy drugs are more likely to cause nausea and vomiting than others. Doctors classify chemotherapy drugs according to their emetogenic potential (how likely the drug will cause nausea or vomiting) as high, moderate, low, or minimal risk.

The GIP receptor agonist peptides of the present disclosure may be used as a preventive/therapeutic agent for vomiting and/or nausea caused, for example, by clinical pathological conditions or causes described in the following (1) to (8). Additionally, the GIP receptor agonist peptide of the present disclosure may be used as a preventive/therapeutic agent for chronic unexplained nausea and vomiting. The vomiting or nausea also includes imminent unpleasant sensations of wanting to eject the contents of the stomach through the mouth such as feeling queasy and retching, and may also be accompanied by autonomic symptoms such as facial pallor, cold sweat, salivary secretion, tachycardia, and diarrhea. The vomiting also includes acute vomiting, protracted vomiting, and anticipatory vomiting.

(1) Diseases accompanied by vomiting or nausea such as gastroparesis, functional dyspepsia, gastrointestinal hypomotility, peritonitis, abdominal tumor, constipation, gastrointestinal obstruction, chronic intestinal pseudo-obstruction, cyclic vomiting syndrome, chronic unexplained nausea and vomiting, acute pancreatitis, chronic pancreatitis and hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);

(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;

(3) Vomiting or nausea caused by radiation sickness or radiation therapy for the chest, the abdomen, or the like used to treat cancers;

(4) Vomiting or nausea caused by a poisonous substance or a toxin;

(5) Vomiting and nausea caused by pregnancy including hyperemesis gravidarium; and

(6) Vomiting and nausea caused by a vestibular disorder such as motion sickness or dizziness

(7) Opioid withdrawal;

(8) Pregnancy including hyperemesis gravidarium;

(9) A vestibular disorder such as motion sickness or dizziness; and

(10) A physical injury causing local, systemic, acute or chronic pain.

These causes of emesis, or nausea, or vomiting are not meant to be exhaustive. Other conditions, activities, side effects may cause emesis, for example, nausea and/or vomiting. Nausea can be measured in ways known to the art, such as through the use of a visual analog scale (VAS).

### D. FORMULATIONS

A medicament containing a GIP receptor agonist peptide of the present disclosure shows low toxicity and is obtained using the compound of the present disclosure alone or in admixture with a pharmacologically acceptable carrier according to a method known per se (e.g., the method described in the Japanese Pharmacopoeia) generally used as production methods of pharmaceutical preparations, and safely administered orally or parenterally (e.g., topically, rectally, subcutaneously, intravenously administered) as a pharmaceutical preparation, for example, tablets (inclusive of sugar-coated tablets, film-coated tablets, sublingual tablets, orally disintegrating tablets), powders, granules, capsules (inclusive of soft capsules, microcapsules), liquids, troches, syrups, emulsions, suspensions, injections (e.g., subcutaneous injections, intravenous injections, intramuscular injections, intraperitoneal injections etc.), external preparations (e.g., transnasal preparations, dermal preparations, ointments), suppository (e.g., rectal suppositories, vaginal suppositories), pellets, nasal preparations, pulmonary preparations (inhalants), transfusions and the like.

These preparations may be controlled release preparations such as a rapid release preparation, a sustained release preparation and the like (e.g., a sustained release microcapsule). The content of the compound of the present disclosure in a pharmaceutical preparation is about 0.01-about 100 wt % of the whole preparation.

The above-mentioned pharmaceutically acceptable carrier may be exemplified by various organic or inorganic carrier materials that are conventionally used as preparation materials, for example, excipient, lubricant, binding agent and disintegrant for solid preparations; or solvent, solubilizing agent, suspending agent, isotonic agent, buffering agent, soothing agent and the like for liquid preparations. Further, if necessary, general additives such as preservative, antioxidant, colorant, sweetening agent, adsorbing agent, wetting agent and the like can be also used appropriately in a suitable amount.

Examples of the excipient include lactose, sucrose, D-mannitol, starch, corn starch, crystalline cellulose, light anhydrous silicic acid and the like.

Examples of the lubricant include magnesium stearate, calcium stearate, talc, colloidal silica and the like.

Examples of the binding agent include crystalline cellulose, sucrose, D-mannitol, dextrin, hydroxypropylcellulose, hydroxypropylmethylcellulose, polyvinylpyrrolidone, starch, sucrose, gelatin, methylcellulose, carboxymethylcellulose sodium and the like.

Examples of the disintegrant include starch, carboxymethylcellulose, carboxymethylcellulose calcium, carboxymethylstarch sodium, L-hydroxypropylcellulose and the like.

Examples of the solvent include water for injection, alcohol, propylene glycol, Macrogol, sesame oil, corn oil, olive oil and the like.

Examples of the solubilizing agent include polyethylene glycol, propylene glycol, D-mannitol, benzyl benzoate, ethanol, trisaminomethane, cholesterol, triethanolamine, sodium carbonate, sodium citrate and the like.

Examples of the suspending agent include surfactants such as stearyl triethanolamine, sodium lauryl sulfate, laurylaminopropionic acid, lecithin, benzalkonium chloride, benzetonium chloride, glycerin monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like; and the like.

Examples of the isotonic agent include glucose, D-sorbitol, sodium chloride, glycerin, D-mannitol and the like.

Examples of the buffering agent include buffer solutions such as phosphates, acetates, carbonates, citrates and the like.

Examples of the soothing agent include benzyl alcohol and the like.

Examples of the preservative include parahydroxybenzoic acid esters, chlorobutanol, benzyl alcohol, phenethyl alcohol, dehydroacetic acid, sorbic acid and the like.

Examples of the antioxidant include sulfites, ascorbic acid, α-tocopherol and the like.

Examples of the colorant include water-soluble food coal tar dyes (e.g., food dyes such as Food Red No. 2 and No. 3, Food Yellow No. 4 and No. 5, Food Blue No. 1 and No. 2, and the like), water-insoluble lake dyes (e.g., aluminum salts of the aforementioned water-soluble Food coal tar dyes), natural dyes (e.g., β-carotene, chlorophyll, ferric oxide red) and the like.

Examples of the sweetening agent include saccharin sodium, dipotassium glycyrrhizinate, aspartame, stevia and the like.

Examples of the adsorbing include porous starch, calcium silicate (trade name: Florite RE), magnesium alumino metasilicate (trade name: Neusilin) and light anhydrous silicic acid (trade name: Sylysia).

Examples of the wetting agent include propylene glycol monostearate, sorbitan monooleate, diethylene glycol monolaurate and polyoxyethylenelauryl ether.

During production of an oral preparation, coating may be applied as necessary for the purpose of masking of taste, enteric property or durability.

Examples of the coating base to be used for coating include sugar coating base, aqueous film coating base, enteric film coating base and sustained-release film coating base.

As the sugar coating base, sucrose is used. Moreover, one or more kinds selected from talc, precipitated calcium carbonate, gelatin, gum arabic, pullulan, carnauba wax and the like may be used in combination.

Examples of the aqueous film coating base include cellulose polymers such as hydroxypropyl cellulose, hydroxypropylmethyl cellulose, hydroxyethyl cellulose, methylhydroxyethyl cellulose etc.; synthetic polymers such as polyvinylacetal diethylaminoacetate, aminoalkyl methacrylate copolymer E [Eudragit E (trade name)], polyvinylpyrrolidone etc.; and polysaccharides such as pullulan etc.

Examples of the enteric film coating base include cellulose polymers such as hydroxypropylmethyl cellulose phthalate, hydroxypropylmethyl cellulose acetate succinate, carboxymethylethyl cellulose, cellulose acetate phthalate etc.; acrylic polymers such as methacrylic acid copolymer L [Eudragit L (trade name)], methacrylic acid copolymer LD [Eudragit L-30D55 (trade name)], methacrylic acid copolymer S [Eudragit S (trade name)] etc.; and naturally occurring substances such as shellac etc.

Examples of the sustained-release film coating base include cellulose polymers such as ethyl cellulose etc.; and acrylic polymers such as aminoalkyl methacrylate copolymer RS [Eudragit RS (trade name)], ethyl acrylate-methyl methacrylate copolymer suspension [Eudragit NE (trade name)] etc.

The above-mentioned coating bases may be used after mixing with two or more kinds thereof at appropriate ratios. For coating, for example, a light shielding agent such as titanium oxide, red ferric oxide and the like can be used.

### E. ADMINISTRATION

The therapeutically effective amount or dose of a composition or medicament containing a GIP receptor agonist peptide to be administered to a subject will depend on the age, sex and weight of the patient, and the current medical condition of the patient. The skilled artisan will be able to determine appropriate dosages depending on these and other factors to achieve the desired biological response.

The dosage of a GIP receptor agonist peptide of the present disclosure is appropriately determined according to the subject of administration, symptom, administration method and the like. For example, when the GIP receptor agonist peptide of the present disclosure is administered orally to a subject prior to engaging in an act that will likely cause emesis or after the onset of emesis in a human subject (body weight of approximately 60 kg), the daily dose of the compound of the present disclosure is about 0.1 to 100 mg, preferably about 1.0 to 50 mg, more preferably about 1.0 to 20 mg. When the compound of the present disclosure is administered parenterally to an obesity or diabetes patient or a gastroparesis (body weight 60 kg), the daily dose of the compound of the present disclosure is about 0.001 to 30 mg, preferably about 0.01 to 20 mg, more preferably about 0.1 to 10 mg. These amounts can be administered in about 1 to several portions a day. In some embodiments, a therapeutically effective amount of a GIP receptor agonist peptide to prevent and/or treat emesis in a subject in need thereof may range from about 0.01 to 0.5 mg/kg/day, 0.1 to 5 mg/kg/day, 5 to 10 mg/kg/day, 10 to 20 mg/kg/day, 20 to 50 mg/kg/day, 10 to 100 mg/kg/day, 10 to 120 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.

The GIP receptor agonist peptide of the present disclosure can be administered, for example, every 2 days, every 3 days, every 4 days, every 5 days, every 6 days, every week, twice per week, every other week, every 3 weeks, every month, every 2 months, every 3 months, every 4 months, every 5 months or every 6 months. In some embodiments, the GIP receptor agonist peptide of the present disclosure can be administered to the subject 1-3 times per day or 1-7 times per week, for 1-5 days, 1-5 weeks, 1-5 months, or 1-5 years.

The GIP receptor agonist peptide of the present disclosure can be used in combination with another drug that does not adversely influence the GIP receptor agonist peptide of the present disclosure, for the purpose of, for example, promoting the action (antiemetic action) of the GIP receptor agonist peptide of the present disclosure, reducing the dose of the GIP receptor agonist peptide of the present disclosure, and the like.

Examples of a drug that can be used in combination with the GIP receptor agonist peptide of the present disclosure (hereinafter sometimes to be abbreviated as a concomitant drug) include anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, therapeutic agents for osteoporosis, vitamins, antidementia drugs, erectile dysfunction drugs, therapeutic drugs for urinary frequency or urinary incontinence, therapeutic agents for dysuria, central D2 receptor antagonists, prokinetic agents, antihistamines, muscarine receptor antagonists, serotonin 5HT₃ receptor antagonists, somatostatin analogues, corticosteroids, benzodiazepine anxiolytics, NK-1 receptor antagonists, hypercalcemia therapeutic drug and the like. Specific examples of the concomitant drug include those mentioned below.

Examples of the anti-obesity agent include monoamine uptake inhibitors (e.g., phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), neuropeptide Y antagonists (e.g., velneperit), cannabinoid receptor antagonists (e.g., rimonabant, taranabant), ghrelin antagonists, ghrelin receptor antagonists, ghrelinacylation enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), Na-glucose cotransporter inhibitors (e.g., JNJ-28431754, remogliflozin), NFK inhibitory (e.g., HE-3286), PPAR agonists (e.g., GFT-505, DRF-11605), phosphotyrosine phosphatase inhibitors (e.g., sodium vanadate, Trodusquemin), GPR119 agonists (e.g., PSN-821, MBX-2982, APD597), glucokinase activators (e.g., AZD-1656), leptin, leptin derivatives (e.g., metreleptin), CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin preparations: FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), GLP-1 receptor agonist, GLP-1 receptor/GIP receptor coagonist, glucagon receptor/GLP-1 receptor/GIP receptor triagonist, and the like.

Here, as the therapeutic agent for diabetes, for example, insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation), insulin sensitizers (e.g., pioglitazone or a salt thereof (preferably, hydrochloride), rosiglitazone or a salt thereof (preferably, maleate), Metaglidasen, AMG-131, Balaglitazone, MBX-2044, Rivoglitazone, Aleglitazar, Chiglitazar, Lobeglitazone, PLX-204, PN-2034, GFT-505, THR-0921, compound described in WO007/013694, WO2007/018314, WO2008/093639 or WO2008/099794), α-glucosidase inhibitors (e.g., voglibose, acarbose, miglitol, emiglitate), biguanides (e.g., metformin, buformin or a salt thereof (e.g., hydrochloride, fumarate, succinate)), insulin secretagogues (e.g., sulfonylurea (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or calcium salt hydrate thereof), dipeptidyl peptidase IV inhibitors (e.g., Alogliptin or a salt thereof (preferably, benzoate), Vildagliptin, Sitagliptin, Saxagliptin, BI1356, GRC8200, MP-513, PF-00734200, PHX1149, SK-0403, ALS2-0426, TA-6666, TS-021, KRP-104, Trelagliptin or a salt thereof (preferably succinate)), β3 agonists (e.g., N-5984), GPR40 agonists (e.g., Fasiglifam or a hydrate thereof, compound described in WO2004/041266, WO2004/106276, WO2005/063729, WO2005/063725, WO2005/087710, WO2005/095338, WO2007/013689 or WO2008/001931), SGLT2 (sodium-glucose cotransporter 2) inhibitors (e.g., Dapagliflozin, AVE2268, TS-033, YM543, TA-7284, Remogliflozin, ASP1941), SGLT1 inhibitors, 11β-hydroxysteroid dehydrogenase inhibitors (e.g., BVT-3498, INCB-13739), adiponectin or agonist thereof, IKK inhibitors (e.g., AS-2868), leptin resistance improving drugs, somatostatin receptor agonists, glucokinase activators (e.g., Piragliatin, AZD1656, AZD6370, TTP-355, compound described in WO006/112549, WO007/028135, WO008/047821, WO008/050821, WO008/136428 or WO008/156757), GPR119 agonists (e.g., PSN821, MBX-2982, APD597), FGF21, FGF analogue, ACC2 inhibitors, GLP-1 receptor agonist, GLP-1 receptor/GIP receptor coagonist, glucagon receptor/GLP-1 receptor/GIP receptor triagonist, and the like can be mentioned.

As the therapeutic agent for diabetic complications may include, aldose reductase inhibitors (e.g., tolrestat, epalrestat, zopolrestat, fidarestat, CT-112, ranirestat (AS-3201), lidorestat), neurotrophic factor and increasing agents thereof (e.g., NGF, NT-3, BDNF, neurotrophic production/secretion promoting agent described in WO01/14372 (e.g., 4-(4-chlorophenyl)-2-(2-methyl-1-imidazolyl)-5-[3-(2-methylphenoxy)propyl]oxazole), compound described in WO2004/039365), PKC inhibitors (e.g., ruboxistaurin mesylate), AGE inhibitors (e.g., ALT946, N-phenacylthiazolium bromide (ALT766), EXO-226, Pyridorin, pyridoxamine), GABA receptor agonists (e.g., gabapentin, pregabalin), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), apoptosis signal regulating kinase-1 (ASK-1) inhibitors, GLP-1 receptor agonist, GLP-1 receptor/GIP receptor coagonist, glucagon receptor/GLP-1 receptor/GIP receptor triagonist, and the like can be mentioned.

As the therapeutic agent for hyperlipidemia, HMG-CoA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compound described in WO97/10224, for example, N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid), fibrate compounds (e.g., bezafibrate, clofibrate, simfibrate, clinofibrate), anion exchange resin (e.g., colestyramine), probucol, nicotinic acid drugs (e.g., nicomol, niceritrol, niaspan), ethyl icosapentate, phytosterol (e.g., soysterol, gamma oryzanol (γ-oryzanol)), cholesterol absorption inhibitors (e.g., zechia), CETP inhibitors (e.g., dalcetrapib, anacetrapib), ω-3 fatty acid preparations (e.g., ω-3-fatty acid ethyl esters 90 (ω-3-acid ethyl esters 90)) and the like can be mentioned.

Examples of the antihypertensive agent include angiotensin converting enzyme inhibitors (e.g., captopril, enalapril, delapril, etc.), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil, etc.), calcium antagonists (e.g., manidipine, nifedipine, amlodipine, efonidipine, nicardipine, cilnidipine, etc.), β blockers (e.g., metoprolol, atenolol, propranolol, carvedilol, pindolol, etc.), clonidine and the like.

As the diuretic, for example, xanthine derivatives (e.g., theobromine sodium salicylate, theobromine calcium salicylate and the like), thiazide preparations (e.g., ethiazide, cyclopenthiazide, trichloromethiazide, hydrochlorothiazide, hydroflumethiazide, benzylhydrochlorothiazide, penfluthiazide, poly5thiazide, methyclothiazide and the like), antialdosterone preparations (e.g., spironolactone, triamterene and the like), carbonic anhydrase inhibitors (e.g., acetazolamide and the like), chlorobenzenesulfonamide agents (e.g., chlortalidone, mefruside, indapamide and the like), azosemide, isosorbide, ethacrynic acid, piretanide, bumetanide, furosemide and the like can be mentioned.

Examples of the chemotherapeutic include alkylating agents (e.g., cyclophosphamide, ifosfamide), antimetabolites (e.g., methotrexate, 5-fluorouracil), anticancer antibiotics (e.g., mitomycin, adriamycin), plant-derived anticancer agents (e.g., vincristine, vindesine, Taxol), cisplatin, carboplatin, etoposide and the like. Among others, a 5-fluorouracil derivative Furtulon or Neofurtulon or the like is preferable. Also a composition comprising a GIP receptor agonist peptide of the disclosure can be administered before, after or during the administration of the following anti-cancer agents: cisplatin, carboplatin. Oxaliplatin, cyclophosphamide, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil.

Examples of the immunotherapeutic include microbial or bacterial components (e.g., muramyl dipeptide derivative, Picibanil), polysaccharides having immunoenhancing activity (e.g., lentinan, sizofiran, Krestin), cytokines obtained by genetic engineering approaches (e.g., interferon, interleukin (IL)), colony-stimulating factors (e.g., granulocyte colony-stimulating factor, erythropoietin) and the like. Among others, interleukins such as IL-1, IL-2, IL-12 and the like are preferable.

Examples of the anti-inflammatory drug include nonsteroidal anti-inflammatory drugs such as aspirin, acetaminophen, indomethacin and the like.

As the antithrombotic agent, for example, heparin (e.g., heparin sodium, heparin calcium, enoxaparin sodium, dalteparin sodium), warfarin (e.g., warfarin potassium), anti-thrombin drugs (e.g., aragatroban, dabigatran), FXa inhibitors (e.g., rivaroxaban, apixaban, edoxaban, YM150, compound described in WO02/06234, WO2004/048363, WO2005/030740, WO2005/058823 or WO2005/113504), thrombolytic agents (e.g., urokinase, tisokinase, alteplase, nateplase, monteplase, pamiteplase), platelet aggregation inhibitors (e.g., ticlopidine hydrochloride, clopidogrel, prasugrel, E5555, SHC530348, cilostazol, ethyl icosapentate, beraprost sodium, sarpogrelate hydrochloride) and the like can be mentioned.

Examples of the therapeutic agent for osteoporosis include alfacalcidol, calcitriol, elcatonin, calcitonin salmon, estriol, ipriflavone, pamidronate disodium, alendronate sodium hydrate, incadronate disodium, risedronate disodium and the like.

Examples of the vitamin include vitamin B₁, vitamin B₁₂ and the like.

Examples of the antidementia drug include tacrine, donepezil, rivastigmine, galanthamine and the like.

Examples of the erectile dysfunction drug include apomorphine, sildenafil citrate and the like.

Examples of the therapeutic drug for urinary frequency or urinary incontinence include flavoxate hydrochloride, oxybutynin hydrochloride, propiverine hydrochloride and the like.

Examples of the therapeutic agent for dysuria include acetylcholine esterase inhibitors (e.g., distigmine) and the like.

Examples of the central D2 receptor antagonist include typical psychotropic drugs (prochlorperazine, haloperidol, chlorpromazine, and the like), serotonin dopamine antagonists (perospirone, risperidone, and the like), and multi-acting receptor targeted antipsychotic drugs (olanzapine and the like).

Examples of the prokinetic agent include peripheral D2 receptor antagonists (metoclopramide, domperidone, and the like) and 5HT4 receptor agonists (mosapride and the like).

Examples of the antihistamine include hydroxyzine, diphenhydramine, and chlorpheniramine.

Examples of the muscarinic receptor antagonist include central muscarinic receptor antagonists (scopolamine and the like) and peripheral muscarinic receptor antagonists (butylscopolamine and the like).

Examples of the serotonin 5HT3 receptor antagonist include granisetron, ondansetron, azasetron, indisetron, palonosetron, and ramosetron.

Examples of the somatostatin analogue include octreotide.

Examples of the corticosteroid include dexamethasone, betamethasone, and methylprednisolone.

Examples of the benzodiazepine anxiolytic include lorazepam and alprazolam, examples of the NK-1 receptor antagonist include aprepitant and fosaprepitant, and examples of the hypercalcemia therapeutic drug include bisphosphonate.

Moreover, a drug confirmed to have a cachexia-ameliorating action either in animal models or clinically, i.e., a cyclooxygenase inhibitor (e.g., indomethacin), a progesterone derivative (e.g., megestrol acetate), glucocorticoid (e.g., dexamethasone), a metoclopramide drug, a tetrahydrocannabinol drug, an agent for improving fat metabolism (e.g., eicosapentaenoic acid), growth hormone, IGF-1, or an antibody against a cachexia-inducing factor TNF-α, LIF, IL-6 or oncostatin M or the like can also be used in combination with the compound of the present disclosure.

Alternatively, a glycation inhibitor (e.g., ALT-711), a nerve regeneration-promoting drug (e.g., Y-128, VX853, prosaptide), an antidepressant (e.g., desipramine, amitriptyline, imipramine), an antiepileptic drug (e.g., lamotrigine, Trileptal, Keppra, Zonegran, Pregabalin, Harkoseride, carbamazepine), an antiarrhythmic drug (e.g., mexiletine), an acetylcholine receptor ligand (e.g., ABT-594), an endothelin receptor antagonist (e.g., ABT-627), a monoamine uptake inhibitor (e.g., tramadol), a narcotic analgesic (e.g., morphine), a GABA receptor agonist (e.g., gabapentin, MR preparation of gabapentin), an α2 receptor agonist (e.g., clonidine), a local analgesic (e.g., capsaicin), an antianxiety drug (e.g., benzothiazepine), a phosphodiesterase inhibitor (e.g., sildenafil), a dopamine receptor agonist (e.g., apomorphine), midazolam, ketoconazole or the like may be used in combination with the compound of the present disclosure.

The time of administration of the GIP receptor agonist peptide of the present disclosure and that of the concomitant drug are not limited, and they may be administered simultaneously or in a staggered manner to the administration subject.

Examples of such administration mode include the following:

(1) administration of a single preparation obtained by simultaneously processing the GIP receptor agonist peptide of the present disclosure and the concomitant drug, (2) simultaneous administration of two kinds of preparations of the GIP receptor agonist peptide of the present disclosure and the concomitant drug, which have been separately produced, by the same administration route, (3) administration of two kinds of preparations of the GIP receptor agonist peptide of the present disclosure and the concomitant drug, which have been separately produced, by the same administration route in a staggered manner, (4) simultaneous administration of two kinds of preparations of the GIP receptor agonist peptide of the present disclosure and the concomitant drug, which have been separately produced, by different administration routes, (5) administration of two kinds of preparations of the compound of the present disclosure and the concomitant drug, which have been separately produced, by different administration routes in a staggered manner (e.g., administration in the order of the GIP receptor agonist peptide of the present disclosure and the concomitant drug, or in the reverse order) and the like.

The dose of the concomitant drug can be appropriately determined based on the dose employed in clinical situations. The mixing ratio of the GIP receptor agonist peptide of the present disclosure and a concomitant drug can be appropriately determined depending on the administration subject, symptom, administration method, target disease, combination and the like. When the subject of administration is human, for example, a concomitant drug can be used in 0.01-100 parts by weight relative to 1 part by weight of the GIP receptor agonist peptide of the present disclosure.

By combining the GIP receptor agonist peptide of the present disclosure and concomitant drug: (1) the dose of the compound of the present disclosure or a concomitant drug can be reduced as compared to single administration of the compound of the present disclosure or a concomitant drug,

(2) the drug to be used in combination with the compound of the present disclosure can be selected depending on the condition of patients (mild, severe and the like),
(3) the period of treatment can be set longer by selecting a concomitant drug having different action and mechanism from those of the compound of the present disclosure,
(4) a sustained treatment effect can be designed by selecting a concomitant drug having different action and mechanism from those of the compound of the present disclosure, and
(5) a synergistic effect can be afforded by a combined use of the compound of the present disclosure and a concomitant drug, and the like, can be achieved.

### F. EXAMPLES

The abbreviations used in the present specification mean the following (Table 1). A hyphen in terms such as α-MePhe and the like as described herein may be omitted, and the event of omission also represents the same meaning.

In the amino acid sequences used in the present specification, the left terminal represents N terminal and the right terminal represents C terminal.

TABLE 1
List of common abbreviations used throughout the specification.

| | |
|---|---|
| Ac | acetyl |
| Aib | α-aminoisobutyric acid |
| Ambz (4) | 4-aminomethylbenzoyl |
| GABA | γ-aminobutyric acid |
| Iva | isovaline |
| Lys (Ac) | Nϵ-acetyllysine |
| α-MePhe | α-methylphenylalanine |
| MeTyr | N-Methyltyrosine |
| Hda | 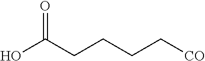 |
| Doda | 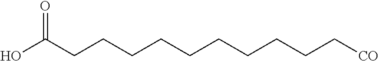 |
| Trda | 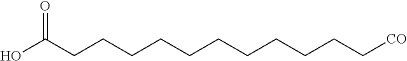 |
| Teda | 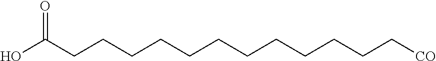 |
| Peda | 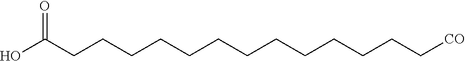 |
| Heda | 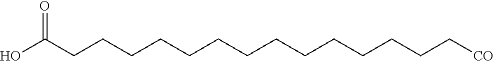 |
| Hepda | 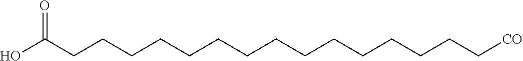 |
| Oda | 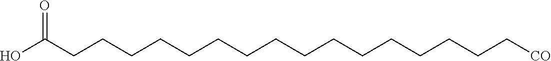 |
| Eda | 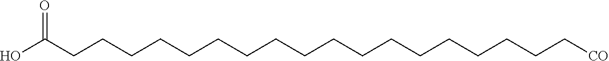 |
| Dda | 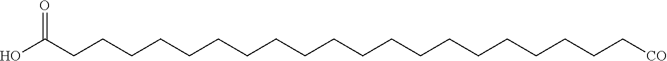 |
| Pal | 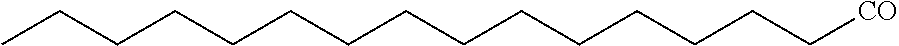 |
| PEG (2) | 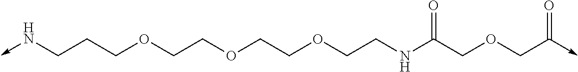 |
| PEG3 | 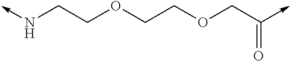 |
| (PEG3) 2 | 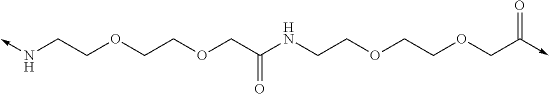 |
| (PEG3) 3 | 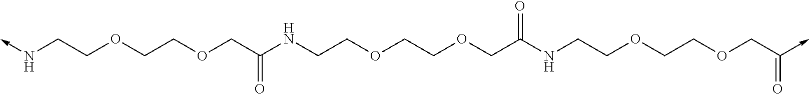 |
| (PEG3) 4 | 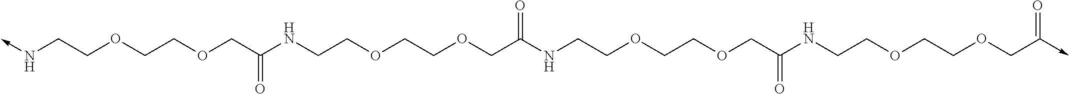 |
| (PEG3) 5 | 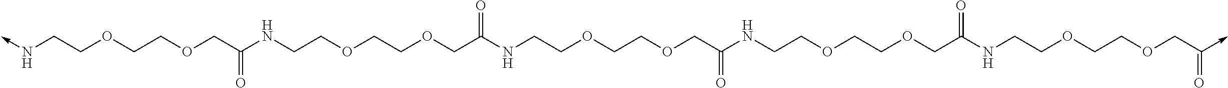 |
| PEG (4) | 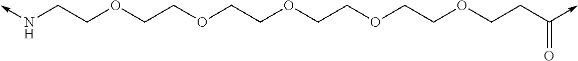 |
| γGlu | 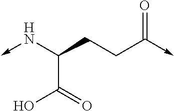 |
| (γGlu) 2 | 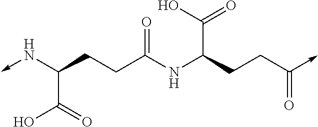 |
| (γGlu) 3 | 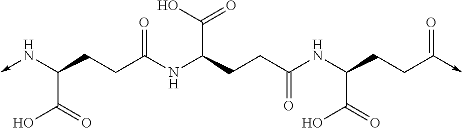 |
| (γGlu) 2- PEG3 | 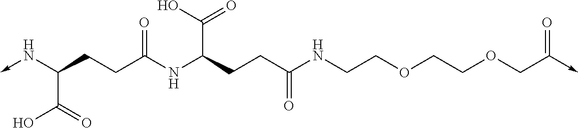 |
| AMBZ-PEG3 | 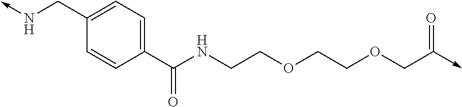 |
| GABA- (PEG3) 2 | 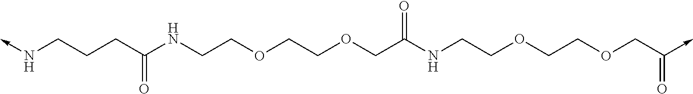 |
| GABA-GGG | 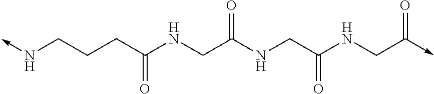 |
| GG | 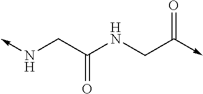 |
| GGG | 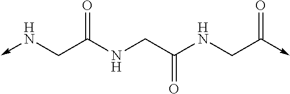 |
| GGGG | 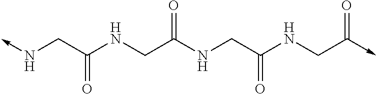 |
| GGGGG | 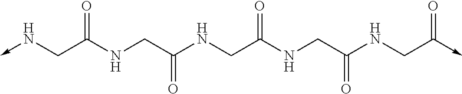 |
| GGGGGG | 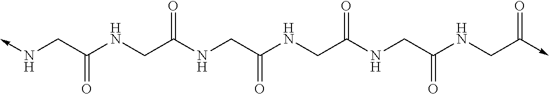 |
| G9 | 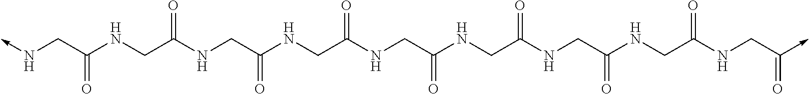 |
| NpipAc | 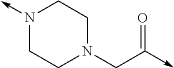 |
| NpipAc- PEG3 | 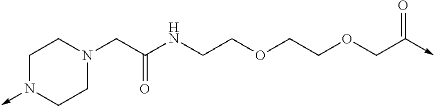 |
| Tra | 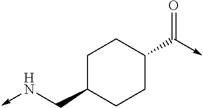 |
| Tra-GGG | 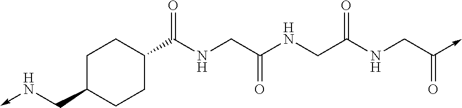 |
| Tra-PEG3 | 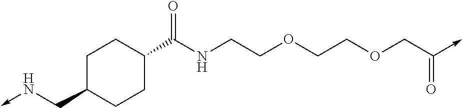 |
| γGlu-PEG3 | 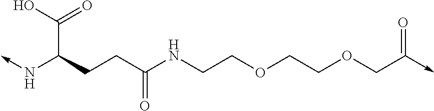 |
| γGlu- (PEG3) 2 | 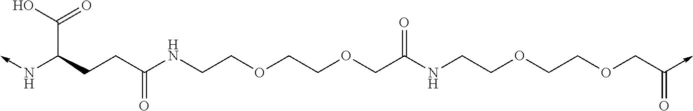 |
| γGlu-AMBZ- PEG3 | 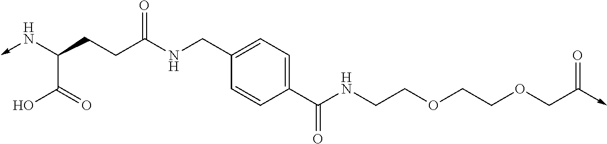 |
| γGlu-GGG | 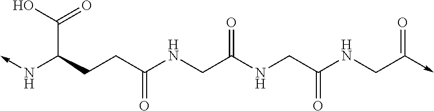 |
| ϵLys | 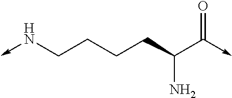 |
| ϵLys-GGG | 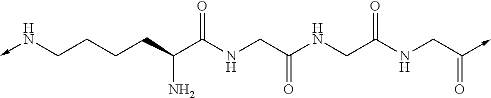 |
| ϵLys-PEG3 | 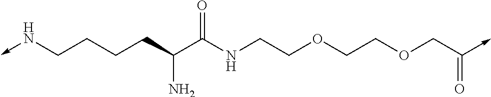 |

In the specification, where bases, amino acids, etc. are denoted by their codes, they are based on conventional codes in accordance with the IUPAC-IUB Commission on Biochemical Nomenclature or by the common codes in the art, examples of which are shown below. For amino acids that may have an optical isomer, L-form is presented unless otherwise indicated (e.g., “Ala” is L-form of Ala). In addition, “D-” means a D-form (e.g., “D-Ala” is D-form of Ala), and “DL-” means a racemate of a D-form and an L-form (e.g., “DL-Ala” is DL racemate of Ala).

The present disclosure is explained in detail in the following by referring to the following Reference Examples, Examples, Test Examples and Formulation Examples, which are mere embodiments and not to be construed as limitative. In addition, the present disclosure may be modified without departing from the scope of invention.

The term “room temperature” in the following Examples indicates the range of generally from about 10° C. to about 35° C. As for “%”, the yield is in mol/mol %, the solvent used for chromatography is in % by volume and other “%” is in % by weight.

NMP: methylpyrrolidone
THF: tetrahydrofuran

### DMF: N,N-dimethylformamide

WSC: 1-(3-dimethylaminopropyl)-3-ethylcarbodiimide hydrochloride

### DCC: N,N′-Dicyclohexylcarbodiimide

DIPCDI: N,N′-diisopropylcarbodiimide
HOBt: 1-hydroxybenzotriazole monohydrate
Oxyma: ethyl 2-cyano-2-(hydroxyimino)acetate

### Example 1

Synthesis of YAibEGTFISDYSIAMDKK(g-Glu-C₁₆)HQQDFVNWLLAQKGKKNDWKHNIIQ (SEQ ID NO: 8, Compound 5)

The peptide was synthesized using standard Fmoc chemistry.

To the 1-chloro-2-[chloro(diphenyl)methyl]benzene (0.400 mmol, 1.00 eq, 0.500 mmol/g) was added FMOC-GLN(TRT)-OH (244.28 mg, 400.00 umol, 1 eq) and DIEA (310 mg, 2.40 mmol, 418 uL, 6.00 eq) in DCM (10.0 mL). The mixture was agitated with N2 for 2 h at 25° C., then added MeOH (0.800 mL) and agitated with N2 for another 30 min. The resin was washed with DMF (30.0 mL*5).

Then 20% piperidine in DMF (30.0 mL) was added and the mixture was agitated with N₂ for 20 min at 25° C. Then the mixture was filtered to get the resin. The resin was washed with DMF (30.0 mL*5) and filtered to get the resin.

Add Fmoc-amino acid solution and mix 30 seconds, then add activation buffer, N2 bubbling for about 0.5 hour.

Repeat step 2 to 3 for next amino acid coupling.

Deprotection: Dde was treated with Hydrazine hydrate/DMF (3/97) 30 mL for 20 min twice, the resin was then washed with DMF (30.0 mL repeated 5 times).

Coupling: Boc was coupling with Boc2O/DIEA/DMF (10:5:85) 15.0 mL for 20 min twice, the resin was then washed with DMF (30.0 mL*5).

Repeat Step 2 and 3 for all other amino acids: (S)-4-((((9H-fluoren-9-yl)methoxy)carbonyl)amino)-5-(tert-butoxy)-5-oxopentanoic acid, 16-(tert-butoxy)-16-oxohexadecanoic acid).

TABLE 2
Reagents and Coupling Reagents Used

| | Materials | Coupling reagents |
|---|---|---|
| 1 | FMOC-GLN(Trt)-OH (1.0 eq) | DIEA(6.0 eq) |
| 2 | FMOC-THR(TBU)-OH (3.0 eq) | HBTU(2.85 eq) and DIEA(6.0 eq) |
| 3 | FMOC-ILE-OH (3.0 eq) | HBTU(2.85 eq) and DIEA(6.0 eq) |
| 4 | FMOC-ASN(Trt)-OH (3.0 eq) | HBTU(2.85 eq) and DIEA(6.0 eq) |
| 5 | FMOC-HIS(Trt)-OH (3.0 eq) | HBTU(2.85 eq) and DIEA(6.0 eq) |
| 6 | FMOC-LYS(BOC)—OH (3.0 eq) | HBTU(2.85 eq) and DIEA(6.0 eq) |
| 7 | FMOC-TRP(BOC)—OH (3.0 eq) | HBTU(2.85 eq) and DIEA(6.0 eq) |
| 8 | FMOC-ASP(OTBU)-OH (3.0 eq) | HBTU(2.85 eq) and DIEA(6.0 eq) |
| 9 | FMOC-ASN(Trt)-OH (3.0 eq) | HBTU(2.85 eq) and DIEA(6.0 eq) |
| 10 | FMOC-LYS(BOC)—OH (3.0 eq) | HBTU(2.85 eq) and DIEA(6.0 eq) |
| 11 | FMOC-LYS(BOC)—OH (3.0 eq) | HBTU(2.85 eq) and DIEA(6.0 eq) |
| 12 | FMOC-GLY-OH (3.0 eq) | HBTU(2.85 eq) and DIEA(6.0 eq) |
| 13 | FMOC-LYS(BOC)—OH (3.0 eq) | HBTU(2.85 eq) and DIEA(6.0 eq) |
| 14 | FMOC-GLN(Trt)-OH (3.0 eq) | HBTU(2.85 eq) and DIEA(6.0 eq) |
| 15 | FMOC-ALA-OH (3.0 eq) | HBTU(2.85 eq) and DIEA(6.0 eq) |
| 16 | FMOC-LEU-OH (3.0 eq) | HATU(2.85 eq) and DIEA(6.0 eq) |
| 17 | FMOC-LEU-OH (3.0 eq) | HATU(2.85 eq) and DIEA(6.0 eq) |
| 18 | FMOC-TRP(BOC)—OH (3.0 eq) | HATU(2.85 eq) and DIEA(6.0 eq) |
| 19 | FMOC-ASN(Trt)-OH (3.0 eq) | HATU(2.85 eq) and DIEA(6.0 eq) |
| 20 | FMOC-VAL-OH (3.0 eq) | HATU(2.85 eq) and DIEA(6.0 eq) |
| 21 | FMOC-PHE-OH (3.0 eq) | HATU(2.85 eq) and DIEA(6.0 eq) |
| 22 | FMOC-ASP(OTBU)-OH (3.0 eq) | HATU(2.85 eq) and DIEA(6.0 eq) |
| 23 | FMOC-GLN(Trt)-OH (3.0 eq) | HATU(2.85 eq) and DIEA(6.0 eq) |
| 24 | FMOC-GLN(Trt)-OH (3.0 eq) | HATU(2.85 eq) and DIEA(6.0 eq) |
| 25 | FMOC-HIS(Trt)-OH (3.0 eq) | HATU(2.85 eq) and DIEA(6.0 eq) |
| 26 | FMOC-LYS(DDE)-OH (3.0 eq) | HATU(2.85 eq) and DIEA(6.0 eq) |
| 27 | FMOC-LYS(BOC)—OH (3.0 eq) | HATU(2.85 eq) and DIEA(6.0 eq) |
| 28 | FMOC-ASP(OTBU)-OH (3.0 eq) | HATU(2.85 eq) and DIEA(6.0 eq) |
| 29 | FMOC-MET-OH (3.0 eq) | HATU(2.85 eq) and DIEA(6.0 eq) |
| 30 | FMOC-ALA-OH (3.0 eq) | HATU(2.85 eq) and DIEA(6.0 eq) |
| 31 | FMOC-ILE-OH (3.0 eq) | HATU(2.85 eq) and DIEA(6.0 eq) |
| 32 | FMOC-SER(TBU)-OH (3.0 eq) | HATU(2.85 eq) and DIEA(6.0 eq) |
| 33 | FMOC-TYR(TBU)-OH (3.0 eq) | HATU(2.85 eq) and DIEA(6.0 eq) |
| 34 | FMOC-ASP(OTBU)-OH (3.0 eq) | HATU(2.85 eq) and DIEA(6.0 eq) |
| 35 | FMOC-SER(TBU)-OH (3.0 eq) | HATU(2.85 eq) and DIEA(6.0 eq) |
| 36 | FMOC-ILE-OH (3.0 eq) | HATU(2.85 eq) and DIEA(6.0 eq) |
| 37 | FMOC-PHE-OH (3.0 eq) | HATU(2.85 eq) and DIEA(6.0 eq) |
| 38 | FMOC-THR(TBU)-OH (3.0 eq) | HATU(2.85 eq) and DIEA(6.0 eq) |
| 39 | FMOC-GLY-OH (3.0 eq) | HATU(2.85 eq) and DIEA(6.0 eq) |
| 40 | FMOC-GLU(OTBU)-OH (3.0 eq) | HATU(2.85 eq) and DIEA(6.0 eq) |
| 41 | FMOC-AIB-OH (3.0 eq) | HATU(2.85 eq) and DIEA(6.0 eq) |
| 42 | FMOC-TYR(TBU)-OH (3.0 eq) | HATU(2.85 eq) and DIEA(6.0 eq) |
| 43 | Boc2O/DIEA/DMF(10:5:85) | Boc2O/DIEA/DMF(10:5:85) |
| 44 | FMOC-GLU-OtBu (3.0 eq) | HATU(2.85 eq) and DIEA(6.0 eq) |
| 45 | C16DA (3.0 eq) | HATU(2.85 eq) and DIEA(6.0 eq) |

TABLE 3
Purification conditions:
Purification condition

| | |
|---|---|
| Dissolution condition | Dissolve in 15% ACN-H₂O |
| Instrument | Gilson GX-281 |
| Mobile Phase | A: H₂O (0.075% TFA in H₂O) |
| | B: CH₃CN |
| Gradient | Condition A: 30-60% - 20 mins |
| | Condition B: 35-65% - 20 mins |
| | Condition C: 25-55% - 20 mins |
| | Condition D: 25-50% - 20 mins |
| | Condition E: 50-80% - 20 mins |
| Column | luna, c18, 10 um, 100 A + |
| | Gemini, 5 um, c18, 110 A |
| Flow Rate | 20 mL/Min |
| Wavelength | 214/ |
| | 254 nm |
| Oven Temp. | Room temperature |

Other GIP receptor agonist peptides (SEQ ID NOs: 4-7 and 9-37) are prepared in a similar manner as disclosed above with reference to SEQ ID NO: 8.

TABLE 4
HPLC Retention time and Mass Spectrum observed mass

| | HPLC | | | Calc. | | | SEQ |
|---|---|---|---|---|---|---|---|
| Compound | RT | Mass | MW | M+/3 | Observed | HPLC | ID |
| No. | (min) | Obs | [Calc] | or 4 | Ion | Method | NO: |
| 1 | 7.0 | 1260.4 | 5037.6 | 1259.4 | M+/4 | C | 4 |
| 2 | 10.0 | 1309.2 | 5233.0 | 1308.3 | M+/4 | A | 5 |
| 3 | 7.9 | 1309.2 | 5233.0 | 1308.3 | M+/4 | B | 6 |
| 4 | 8.8 | 1178.6 | 3532.9 | 1177.6 | M+/3 | C | 7 |
| 5 | 9.2 | 1353.5 | 5410.1 | 1352.5 | M+/4 | C | 8 |
| 6 | 7.5 | 1209.7 | 3629.2 | 1209.7 | M+/3 | E | 9 |
| 8 | 9.2 | 1247.9 | 4987.6 | 1246.9 | M+/4 | C | 11 |
| 9 | 7.5 | 1288.9 | 5151.3 | 1287.8 | M+/4 | C | 12 |
| 10 | 10.0 | 1418.2 | 4254.7 | 1418.2 | M+/3 | B | 13 |
| 11 | 7.9 | 1437.4 | 4309.8 | 1436.6 | M+/3 | C | 14 |
| 12 | 7.9 | 1426.7 | 4277.8 | 1425.9 | M+/3 | C | 15 |
| 13 | 11.4 | 1428.7 | 5710.5 | 1427.6 | M+/4 | C | 16 |
| 14 | 10.3 | 1249.7 | 4994.6 | 1248.7 | M+/4 | D | 17 |
| 15 | 9.1 | 1475.6 | 4422.9 | 1474.3 | M+/3 | A | 18 |
| 16 | 10.1 | 1271.5 | 5081.8 | 1270.4 | M+/4 | C | 19 |
| 17 | 10.1 | 1426.7 | 4276.7 | 1425.6 | M+/3 | A | 20 |
| 18 | 11.5 | 1605.6 | 4813.5 | 1604.5 | M+/3 | B | 21 |
| 19 | 9.0 | 1439.1 | 5751.5 | 1437.9 | M+/4 | A | 22 |
| 20 | 10.5 | 1615.4 | 4843.4 | 1614.5 | M+/3 | B | 23 |
| 21 | 9.9 | 1420.8 | 4259.8 | 1419.9 | M+/3 | A | 34 |
| 21 | 11.5 | 1625.1 | 4872.4 | 1624.1 | M+/3 | B | 24 |
| 22 | 9.2 | 1643.8 | 4928.5 | 1642.8 | M+/3 | B | 25 |
| 23 | 8.8 | 1644.0 | 4928.5 | 1642.8 | M+/3 | B | 26 |
| 24 | 9.1 | 1434.7 | 4300.9 | 1433.6 | M+/3 | A | 27 |
| 25 | 10.8 | 1427.3 | 4278.8 | 1426.3 | M+/3 | A | 28 |
| 26 | 7.9 | 1435.3 | 4302.8 | 1434.3 | M+/3 | A | 29 |
| 27 | 7.8 | 1416.0 | 4244.8 | 1414.9 | M+/3 | A | 30 |
| 28 | 9.1 | 1415.3 | 4242.8 | 1414.3 | M+/3 | A | 31 |
| 29 | 8.6 | 1256.0 | 5020.6 | 1255.2 | M+/4 | A | 32 |
| 30 | 10.1 | 1667.3 | 4998.6 | 1666.2 | M+/3 | A | 33 |
| 32 | 8.4 | 1435.4 | 4302.8 | 1434.3 | M+/3 | A | 35 |
| 33 | 9.1 | 1582.1 | 4743.3 | 1581.1 | M+/4 | B | 36 |

Other exemplary methods for synthesizing GIP receptor agonist peptides are disclosed for example in Applicant's International PCT Application No. PCT/JP2018/013540, filed on Mar. 30, 2018, ranging from pages 162 to 213, the disclosure of which is specifically incorporated herein by reference in its entirety.

### Biological Examples

Methods for performing GIP and GLP receptor binding assays, assays for inhibition of emesis, vomiting and nausea, caused by various stimuli, including from drug or chemotherapy induced nemesis are specifically described in Applicant's International PCT Application No. PCT/JP2018/013540, filed on Mar. 30, 2018, ranging from pages 213 to 255, and are specifically incorporated herein by reference in their entirety.

Example 1—(A) Evaluation of agonist activity on human GIPR and human GLP-1R with increase in intracellular cAMP concentration as indicator (1) Construction of expression plasmid of human GIPR gene. The human GIPR gene having an identical sequence to Genebank Accession No. U39231 is cloned into a pMSRα-neo vector to prepare hGIPR/pMSRα-neo.

(2) Construction of Reporter Plasmid Expressing Cell

Luciferase reporter gene with a cAMP-responsive sequence located upstream is transferred to a CHO-K1 cell to construct a CRE-LUC/CHO-K1 cell.

(3) Construction of Reporter Plasmid

Four copies of a cAMP responsive sequence and a Zeocin resistance gene are transferred to pGL3(R2.2)-Basic Vector (Promega) to construct a Cre-luc(Zeo) reporter plasmid.

(4) Transfer of Human GIPR Gene to CRE-LUC/CHO-K1 Cell and Obtaining of Expressing Cell

The plasmid hGIPR/pMSRα-neo obtained in (1) is transferred to the CRE-LUC/CHO-K1 cell obtained in (2) to obtain a transformant. Subsequently, the cell line inducing the luciferase expression, hGIPR/CRE-LUC/CHO-K1 cell, is selected from the obtained transformant by adding GIP.

(5) Construction of Expression Plasmid of Human GLP-1R Gene

The human GLP-1R gene having an identical sequence to Genebank Accession No. NM_002062 is cloned into a pIRESneo3 vector to prepare hGLP-1/pIRESneo3.

(6) Transfer of Human GLP-1R Gene and Reporter Plasmid to CHO-K1 Cell and Obtaining of Expressing Cell

The Cre-luc (Zeo) obtained in (3) and the plasmid hGLP-1/pIRESneo3 obtained in (5) transferred to the CHO-K1 cell to obtain a transformant. Subsequently, the cell line inducing the luciferase expression, hGLP-1R/CRE-luc/CHO-K1 cell, is selected from the obtained transformant by adding GLP-1.

Reporter Assay

25 μL each of the hGIPR/CRE-LUC/CHO-K1 cell is inoculated in a 384-well white plate (Corning) in such a way as to provide 5×10³ cells/well and may be cultured in a HamF12 medium containing 10% fetal bovine serum, 100 U/mL penicillin, and 100 μg/mL streptomycin in a CO2 incubator at 37° C. overnight. 5 μL of a medium containing a test compound is added to the cells and incubated for 4 hours at the indicated concentration in a CO2 incubator at 37° C. Steady-Glo (Promega) is added in amounts of 30 μL and shaken under light shielding. Twenty minutes later, luciferase activity is measured using a plate reader Envision (PerkinElmer). The GIPR agonist activity is calculated with increase in intracellular cAMP concentration as the indicator when the luciferase activity in the presence of 10 nM of GIP was 100% and the luciferase activity in the case of adding DMSO instead of the test compound was 0%.

The GLP-1R agonist activity is assayed in the same manner as above using the hGLP-1R/CRE-luc/CHO-K1 cell. The GLP-1R agonist activity is calculated with increase in intracellular cAMP concentration as the indicator when the luciferase activity in the presence of 10 nM of GLP-1 was 100% and the luciferase activity in the case of adding DMSO instead of the test compound was 0%.

As shown in Table 6, the GIP receptor agonist peptides of the present disclosure have an excellent GIP receptor selective activation action.

TABLE 5
GIP Receptor Selective Activation by Various GIP
receptor agonist peptides of the Disclosure

| | | | GIPR | GLP1R | Selectivity | SEQ |
|---|---|---|---|---|---|---|
| Compound | GIPR | GLP1R | EC₅₀ nM | EC₅₀ nM | Ratio | ID |
| No. | pEC₅₀ | pEC₅₀ | (A) | (B) | (B/A) | NO: |
| 1 | 11.0 | 6.5 | 0.010 | 302.0 | 30,200 | 4 |
| 2 | 10.9 | 8.3 | 0.012 | 5.0 | 417 | 5 |
| 3 | 9.5 | 6.8 | 0.331 | 147.9 | 447 | 6 |
| 4 | 12.4 | 6.5 | 0.001 | 354.8 | >100,000 | 7 |
| 5 | 11.9 | 7.0 | 0.001 | 93.3 | 93,300 | 8 |
| 8 | 10.7 | 6.9 | 0.021 | 117.5 | 5,595 | 11 |
| 9 | 11.4 | <6 | 0.004 | >1000 | >100,000 | 12 |
| 11 | 12.6 | 6.1 | 0.001 | 831.8 | >100,000 | 14 |
| 12 | 10.4 | <6 | 0.042 | >1000 | >23,810 | 15 |
| 13 | 10.62 | 6.29 | 0.024 | 512.9 | 21,369 | 16 |
| 14 | 12.30 | 6.03 | 0.001 | 933.3 | >100,000 | 17 |
| 15 | 10.12 | 10.77 | 0.076 | <0.02 | 0.26 | 18 |
| 16 | 12.00 | 10.77 | 0.001 | <0.02 | 20 | 19 |
| 17 | 12.05 | 10.77 | 0.001 | <0.02 | 20 | 20 |
| 18 | 12.70 | 11.08 | <0.001 | <0.02 | 20 | 21 |
| 19 | 12.15 | 8.26 | 0.001 | 5.495 | 7851 | 22 |
| 20 | 11.82 | 6.37 | 0.002 | 426.6 | >100,000 | 23 |
| 21 | 11.46 | 6.39 | 0.004 | 407.4 | >100,000 | 24 |
| 22 | 12.00 | 8.14 | 0.001 | 7.244 | 7244 | 25 |
| 23 | 12.10 | 8.57 | 0.001 | 2.692 | 3364 | 26 |
| 24 | 12.40 | 7.48 | <0.001 | 33.1 | 82783 | 27 |
| 25 | 12.40 | <8 | <0.001 | >1000 | >100,000 | 28 |
| 26 | 12.15 | <9 | 0.001 | >1000 | >100,000 | 29 |
| 27 | 12.15 | 6.02 | 0.001 | 955.0 | >100,000 | 30 |
| 28 | 12.52 | 7.13 | <0.001 | 74.1 | >100,000 | 31 |
| 29 | 12.70 | 7.01 | <0.001 | 97.7 | >100,000 | 32 |
| 30 | 12.70 | 6.40 | <0.001 | 398.1 | >100,000 | 33 |
| 31 | 11.77 | 6.85 | 0.002 | 141.3 | 83090 | 34 |
| 32 | 11.77 | 6.85 | 0.0017 | 141.3 | 83090 | 35 |
| 33 | 12.52 | 10.77 | <0.001 | <0.02 | 20 | 36 |
| 34 | 12.00 | 10.77 | 0.001 | <0.02 | 20 | 37 |

B) Evaluation of Binding Activity to Human GIPR Using [¹²⁵I]-GIP

(1) Construction of Expression Plasmid of Human GIPR Gene

The human GIPR gene having an identical sequence to Genebank Accession No. U39231 is cloned into a pcDNA3.3 vector to prepare hGIPR/pcDNA3.3.

### Example 2—Preparation of Human GIPR Virus-Like Particle (VLP) Using Expi293F Cell

On the day before transfection, 850 mL of Expi293F cells are inoculated in a concentration of 1.8×10⁶ cells/mL in a 3-L flask (Corning Incorporated) and cultured under conditions of 37° C., 8% CO₂, 85 rpm for 24 hours. The transfection is s carried out using Expi293 Expression System Kit (Thermo Fisher Scientific). More specifically, 0.67 mg of pcDNA3.3/hGIPR and 0.33 mg of pcDNA3.3/GAG plasmid for VLP preparation are added to 50 mL of opti-MEM (Thermo Fisher Scientific) to prepare a DNA mixture. Subsequently, 2.7 mL of Expifectamine is added to 50 mL of opti-MEM and allowed to stand for 5 minutes, then the DNA mixture is mixed thereinto and the resulting mixture is allowed to stand for 20 minutes and then further added to the culture medium. 20 hours after transfection, 5 mL of Enhancers 1 and 50 mL of Enhancers 2 are added thereto. 96 hours after transfection, the culture medium is centrifuged at 850×g for 15 minutes to thereby obtain a supernatant. The obtained supernatant is ultra-centrifuged at 54000×g for 1 hour to thereby obtain a GIPR-VLP fraction. The precipitate is washed once with PBS and then suspended in a small amount of PBS to thereby obtain GIPR-VLP. The obtained GIPR-VLP is stored at −80° C. until used. Protein quantification is carried out using GelCode Blue Safe Protein Stain (Thermo Fisher Scientific) with BSA as the standard.

### Example 3—Measurement of Binding Activity of Test Compounds to Human GIPR

For the measurement of the binding activity to GIPR, [¹²⁵I]GIP (PerkinElmer, Inc.) in a final concentration of 100 pM and a test compound in a specified concentration are mixed to GIPR-VLP in an assay buffer (50 mM HEPES (pH 7.4, WAKO 342-01375), 5 mM EGTA (WAKO 346-01312), 5 mM MgCl₂ (WAKO 136-03995), 0.1% BSA (Merckmillipore 81-066-04) and 0.005% Tween 20 (BioRad 170-6531)) and reacted at room temperature for 2 hours. VLP to which [¹²⁵I]GIP is bound is trapped in a GF/C glass fiber filter 96-well plate (PerkinElmer 6005274) using a cell harvester and washed with an assay buffer. The GF/C glass fiber filter 96-well plate in which VLP is trapped is dried at 42° C. overnight. Thereafter, MicroScint-O (PerkinElmer 6013611) is added to the GF/C glass fiber filter 96-well plate sealed using a backseal and the plate is sealed using a topseal. The radioactivity of each well is eventually measured using Topcount (PerkinElmer) and the binding activity of the test compound to GIPR is calculated when the [¹²⁵I]GIP binding activity in the presence of GIP in a final concentration of 1 μm was 100% and the [¹²⁵I]GIP binding activity of the wells to which DMSO was added was 0%.

### Example 4—Evaluation of Peptide Agonist Activity on Human GIPR and Human GLP1R by Measuring Intracellular cAMP Accumulation

GIPR Assay

HEK-293T cells overexpressing full-length human GIPR with a sequence identical to GenBank accession number NM_000164 with an N-terminal FLAG tag are purchased from Multispan, Inc (Hayward, Calif.). Cells are cultured per the manufacturer's protocol in DMEM with 10% fetal bovine serum and 1 μg/mL puromycin, and stored in frozen aliquots to be used as assay ready cells. On the day of the assay, cells are removed from frozen storage, washed two times in 1× Kreb's Ringer Buffer (Zenbio, Research Triangle Park, N.C.), and re-suspended to a concentration of 4×10⁵ cells/mL in 1× Kreb's Ringer Buffer. 50 nL of test compound in 100% DMSO spanning a final concentration range of 3×10⁻¹⁰-5.08×10⁻¹⁵ M are acoustically dispensed in low volume, white, 384-well polypropylene plates (Corning, Tewksbury, Mass.), followed by the addition of 4×10³ cells per well in total volume of 10 μL. Cells are incubated with test compound for 1 hr at room temperature in the dark, and cAMP accumulation is measured using the Cisbio HiRange cAMP assay kit (Bedford, Mass.) per the manufacturer's protocol. Anti-cAMP antibody and d2-cAMP tracer reagents diluted in lysis/detection buffer are incubated in the dark for 1 hr, and results are measured on an Envision plate reader (Perkin Elmer, Waltham, Mass.). Data is normalized using 1 nM GIP as 100% activity, and DMSO alone as 0% activity.

### Example 6—In-Vivo Effect of GIP Receptor Agonist Peptides on Emesis in Morphine Induced Emesis Model in Live Male Ferrets

Effect of subcutaneously administered GIP receptor agonist peptide in morphine-induced acute emetic model.

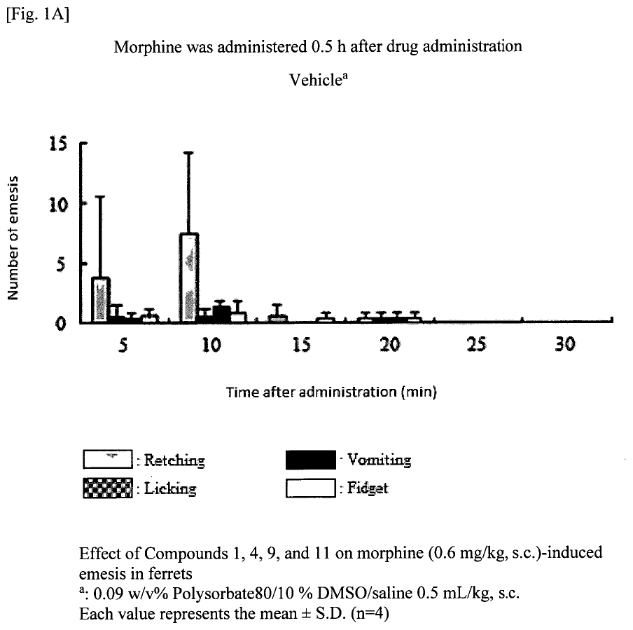

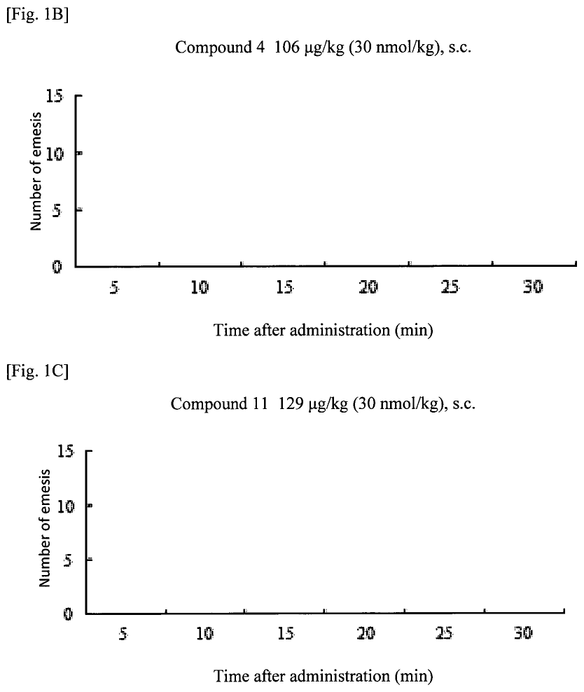

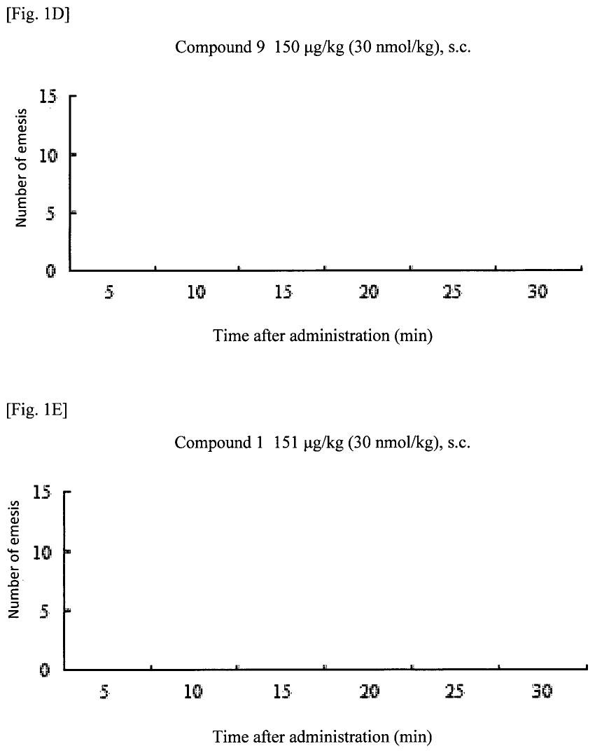

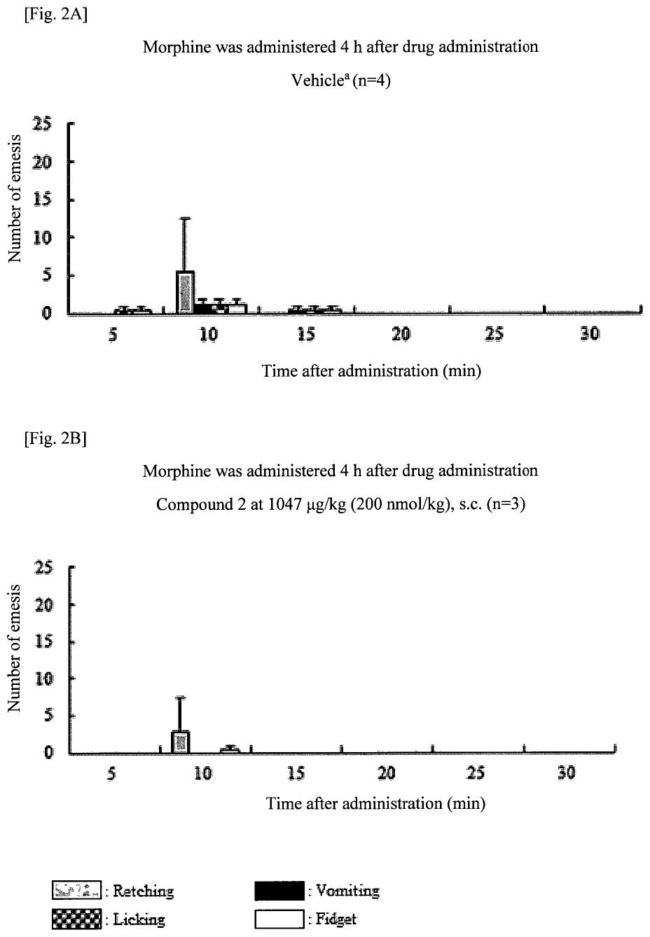

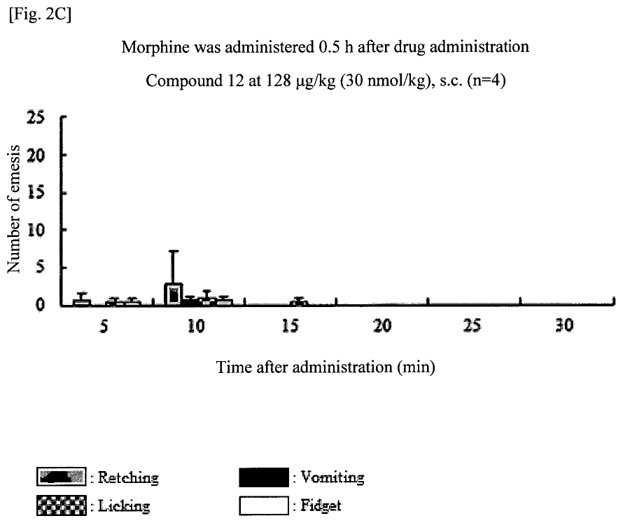

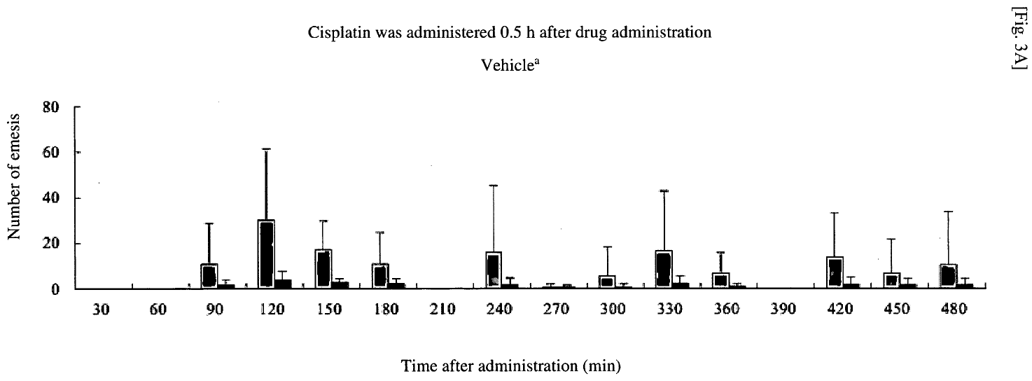

To evaluate the antiemetic effect, the GIP receptor agonist peptides other than natural human GIP were subcutaneously administered into male ferrets 30 minutes before morphine administration. Compounds 1, 4, 9 and 11 in a dose of 30 nmol/kg completely attenuated the morphine (0.6 mg/kg, s.c.)-induced emesis in the ferrets (FIGS. 1A-1E) and compounds 2, and 12 (FIGS. 2A-2C). Up to 60 minutes after morphine administration, the condition of the ferrets was monitored to record the frequencies and time points of abdominal contraction motions, vomiting behaviors, licking with the tongue, and fidgety behavior occurring

Effects of Subcutaneously Administered GIP Receptor Agonist Peptides in Morphine-Induced Acute Emetic Model

30 nmol/kg of Compounds 3 and 5 were dissolved with a vehicle (0.09 w/v % tween 80/10% DMSO/saline), respectively, to prepare test solutions. 0.5 mg/kg of the test solutions and the vehicle were subcutaneously administered to ferrets (4 in each group), respectively. At the time of each of 4 hours, after administration, 0.6 mg/kg of morphine was subcutaneously administered. Up to 60 minutes after morphine administration, the condition of the ferrets was monitored to record the number of animals that did not vomit, the number of emetic episodes, the latency period in minutes to observe the emetic episodes, the duration of the observed emesis if any. See results in Tables 6, 7 and 8.

TABLE 6
Inhibitory effects of 2 GIP receptor agonist peptides on morphine-induced emesis in
male ferrets dosed 4 hours after administration of the GIP receptor agonist peptides.

| | | | No. of | Emetic | | |
|---|---|---|---|---|---|---|
| | | | animals | episodes | Latency | Duration |
| Compound | Dose | | that did not | [% Inhibition | period ^{a)} | of emesis |
| No. | (nmol/kg) | N | vomit | of mean value] | (min) | (min) |
| Vehicle | 0 | 4 | 1 | 7.5 ± 5.1 | 11.5 ± 12.4 | 3.0 ± 3.2 |
| | | | | (Individual value: | | |
| | | | | 0, 10, 11, 9) | | |
| 3 | 30 | 4 | 0 | 21.8 ± 19.2 | 7.0 ± 2.9 | 5.5 ± 7.1 |
| | | | | [−143%] | | |
| 5 | 30 | 3 | 3 | 0.0 ± 0.0 | 30.0 ± 0.0 | 0.0 ± 0.0 |
| | | | | [100%] | | |

TABLE 7
Effect of Compounds 1, 4, 9, 11 on morphine (0.6 mg/kg, s.c.)-induced emesis in ferrets

| Morphine was | | | | No. of animals | Latency^{a} | Duration^{b} | |
|---|---|---|---|---|---|---|---|
| administered | Signs | Drugs | Doses | (responder/used) | (min) | (min) | Frequency |
| 0.5 h | Retching | Vehicle^{c} | — | 4/4 | 5.0 ± 1.4 | 6.3 ± 4.1 | 12.0 ± 11.3 |
| after | | Compound 4 | 106 μg/kg, s.c. | 0/4 # | 30.0 ± 0.0 ** | 0.0 ± 0.0 * | 0.0 ± 0.0 |
| | | Compound 9 | 150 μg/kg, s.c. | 0/4 # | 30.0 ± 0.0 ** | 0.0 ± 0.0 * | 0.0 ± 0.0 |
| | | Compound 11 | 129 μg/kg, s.c. | 0/4 # | 30.0 ± 0.0 ** | 0.0 ± 0.0 * | 0.0 ± 0.0 |
| | | Compound 1 | 151 μg/kg, s.c. | 0/4 # | 30.0 ± 0.0 ** | 0.0 ± 0.0 * | 0.0 ± 0.0 |
| | Vomiting | Vehicle^{c} | — | 2/4 | 17.3 ± 14.8 | 4.0 ± 5.2 | 1.3 ± 1.5 |
| | | Compound 4 | 106 μg/kg, s.c. | 0/4 | 30.0 ± 0.0 | 0.0 ± 0.0 | 0.0 ± 0.0 |
| | | Compound 9 | 150 μg/kg, s.c. | 0/4 | 30.0 ± 0.0 | 0.0 ± 0.0 | 0.0 ± 0.0 |
| | | Compound 11 | 129 μg/kg, s.c. | 0/4 | 30.0 ± 0.0 | 0.0 ± 0.0 | 0.0 ± 0.0 |
| | | Compound 1 | 151 μg/kg, s.c. | 0/4 | 30.0 ± 0.0 | 0.0 ± 0.0 | 0.0 ± 0.0 |
| | Emesis | Vehicle^{c} | — | 4/4 | 5.0 ± 1.4 | 6.3 ± 4.1 | 13.3 ± 12.4 |
| | | Compound 4 | 106 μg/kg, s.c. | 0/4 # | 30.0 ± 0.0 ** | 0.0 ± 0.0 * | 0.0 ± 0.0 |
| | | Compound 9 | 150 μg/kg, s.c. | 0/4 # | 30.0 ± 0.0 ** | 0.0 ± 0.0 * | 0.0 ± 0.0 |
| | | Compound 11 | 129 μg/kg, s.c. | 0/4 # | 30.0 ± 0.0 ** | 0.0 ± 0.0 * | 0.0 ± 0.0 |
| | | Compound 1 | 151 μg/kg, s.c. | 0/4 # | 30.0 ± 0.0 ** | 0.0 ± 0.0 * | 0.0 ± 0.0 |
| | Licking | Vehicle^{c} | — | 4/4 | 6.3 ± 2.9 | 3.5 ± 3.0 | 1.8 ± 0.5 |
| | | Compound 4 | 106 μg/kg, s.c. | 0/4 # | 30.0 ± 0.0 ** | 0.0 ± 0.0 | 0.0 ± 0.0 ** |
| | | Compound 9 | 150 μg/kg, s.c. | 0/4 # | 30.0 ± 0.0 ** | 0.0 ± 0.0 | 0.0 ± 0.0 ** |
| | | Compound 11 | 129 μg/kg, s.c. | 0/4 # | 30.0 ± 0.0 ** | 0.0 ± 0.0 | 0.0 ± 0.0 ** |
| | | Compound 1 | 151 μg/kg, s.c. | 0/4 # | 30.0 ± 0.0 ** | 0.0 ± 0.0 | 0.0 ± 0.0 ** |
| | Fidget | Vehicle^{c} | — | 3/4 | 11.0 ± 12.7 | 6.0 ± 4.5 | 1.8 ± 1.3 |
| | | Compound 4 | 106 μg/kg, s.c. | 0/4 | 30.0 ± 0.0 * | 0.0 ± 0.0 * | 0.0 ± 0.0 * |
| | | Compound 9 | 150 μg/kg, s.c. | 0/4 | 30.0 ± 0.0 * | 0.0 ± 0.0 * | 0.0 ± 0.0 * |
| | | Compound 11 | 129 μg/kg, s.c. | 0/4 | 30.0 ± 0.0 * | 0.0 ± 0.0 * | 0.0 ± 0.0 * |
| | | Compound 1 | 151 μg/kg, s.c. | 0/4 | 30.0 ± 0.0 * | 0.0 ± 0.0 * | 0.0 ± 0.0 * |

Each value represents the mean ± S.D.
^{a}The latency of ferret that did not show emetic response was considered as 30 min.
^{b}The cumulative total duration of emetic response.
^{c}0.09 w/v % Polysorbate 80/10% DMSO/saline 0.5 mL/kg, s.c.
#: Significant difference from vehicle treatment p < 0.05 (Fisher's exact test)
* and **: Significant difference from vehicle treatment p < 0.05 and p < 0.01, respectively (Aspin-Welch's t -test, One-side)

TABLE 8
Effect of Compounds 2 and 12 on morphine (0.6 mg/kg, s.c.)-induced emesis in ferret

| | | | | No. of animals | | | |
|---|---|---|---|---|---|---|---|
| | | | Morphine was | (responder/ | Latency^{a} | Duration^{b} | |
| Signs | Drugs | Doses | administered | used) | (min) | (min) | Frequency |
| Retching | Vehicle^{c} | — | 4 h after | 4/4 | 7.5 ± 1.3 | 1.0 ± 0.0 | 5.5 ± 7.0 |
| | Compound 2 | 1047 μg/kg, s.c. | 4 h after | 1/3 | 22.7 ± 12.7 | 0.3 ± 0.6 | 2.7 ± 4.6 |
| | Compound 12 | 128 μg/kg, s.c. | 0.5 h after | 2/4 | 18.3 ± 13.7 | 1.5 ± 2.4 | 3.3 ± 5.3 |
| Vomiting | Vehicle^{c} | — | 4 h after | 3/4 | 13.0 ± 11.4 | 2.3 ± 3.2 | 1.3 ± 1.0 |
| | Compound 2 | 1047 μg/kg, s.c. | 4 h after | 0/3 | 30.0 ± 0.0 * | 0.0 ± 0.0 | 0.0 ± 0.0 * |
| | Compound 12 | 128 μg/kg, s.c. | 0.5 h after | 2/4 | 19.5 ± 12.1 | 0.5 ± 0.6 | 0.5 ± 0.6 |
| Emesis | Vehicle^{c} | — | 4 h after | 4/4 | 7.5 ± 1.3 | 2.5 ± 3.0 | 6.8 ± 6.9 |
| | Compound 2 | 1047 μg/kg, s.c. | 4 h after | 1/3 | 22.7 ± 12.7 | 0.3 ± 0.6 | 2.7 ± 4.6 |
| | Compound 12 | 128 μg/kg, s.c. | 0.5 h after | 2/4 | 18.3 ± 13.7 | 1.5 ± 2.4 | 3.8 ± 5.7 |
| Licking | Vehicle^{c} | — | 4 h after | 4/4 | 6.3 ± 2.5 | 2.8 ± 2.9 | 1.5 ± 0.6 |
| | Compound 2 | 1047 μg/kg, s.c. | 4 h after | 0/3 # | 30.0 ± 0.0 ** | 0.0 ± 0.0 | 0.0 ± 0.0 ** |
| | Compound 12 | 128 μg/kg, s.c. | 0.5 h after | 3/4 | 13.8 ± 11.3 | 1.5 ± 1.7 | 1.3 ± 1.0 |
| Fidget | Vehicle^{c} | — | 4 h after | 3/4 | 12.3 ± 12.0 | 3.3 ± 3.3 | 1.5 ± 1.3 |
| | Compound 2 | 1047 μg/kg, s.c. | 4 h after | 1/3 | 22.7 ± 12.7 | 0.3 ± 0.6 | 0.3 ± 0.6 |
| | Compound 12 | 128 μg/kg, s.c. | 0.5 h after | 2/4 | 18.3 ± 13.7 | 1.3 ± 1.9 | 0.8 ± 1.0 |

Each value represents the mean ± S.D.
^{a}The latency of ferret that did not show emetic response was considered as 30 min.
^{b}The cumulative total duration of emetic response.
^{c}0.09 w/v % Polysorbate 80/10% DMSO/saline 0.5 mL/kg, s.c.
#: Significant difference from vehicle treatment p < 0.05 (Fisher's exact test)
* and **: Significant difference from vehicle treatment p < 0.05 and p < 0.01, respectively (Aspin-Welch's t -test, One-side)

As a result, in the experiment wherein morphine was administered 4 hours after administration, the vomiting behavior was noted in all of the 4 ferrets in the vehicle group but the vomiting symptom was observed in all of the 4 ferrets in each of compounds 3 and 7, but none of the three ferrets of the four in compound 5.

Effects of Subcutaneously Administered Compound 33, Compound 25 and 22 in Morphine-Induced Acute Emetic Model

10 nmol/kg of each compound dissolved with a vehicle (0.09 w/v % tween 80/10% DMSO/saline) respectively, to prepare test solutions. 0.5 mg/kg of the test solutions and the vehicle were subcutaneously administered to ferrets (4 in each group), respectively. At the time of each of 4 hours, or 120 hours after administration, 0.6 mg/kg of morphine was subcutaneously administered. Up to 360 minutes after morphine administration, the condition of the ferrets was monitored to record the frequencies and time points of abdominal contraction motions, vomiting behaviors, licking with the tongue, and fidgety behavior occurring.

Tables 9 and 10. Summary of the effects of Compound 33, Compound 25 and Compound 20 on morphine (0.6 mg/kg, s.c.)-induced emesis in ferrets (Summary Table 9, Individual Results Table 10)

TABLE 9
Summary of the effects of Compound 33, Compound 25 and Compound
20 on morphine (0.6 mg/kg, s.c.)-induced emesis in ferrets

| Morphine was | | | Dose | No. of animals | Latency^{a} | Duration^{b} | |
|---|---|---|---|---|---|---|---|
| administered | Signs | Drugs | (nmol/kg, s.c.) | (responder/used) | (min) | (min) | Frequency |
| 4 h | Retching | Vehicle^{c} | — | 4/4 | 3.8 ± 1.7 | 12.3 ± 9.3 | 38.0 ± 12.6 |
| after | | Compound 33 | 10 | 0/4 # | 30.0 ± 0.0 ** | 0.0 ± 0.0 * | 0.0 ± 0.0 ** |
| | | Compound 25 | 10 | 0/4 # | 30.0 ± 0.0 ** | 0.0 ± 0.0 * | 0.0 ± 0.0 ** |
| | | Compound 20 | 10 | 0/4 # | 30.0 ± 0.0 ** | 0.0 ± 0.0 * | 0.0 ± 0.0 ** |
| | Vomiting | Vehicle^{c} | — | 4/4 | 6.3 ± 2.4 | 5.0 ± 3.6 | 3.0 ± 2.2 |
| | | Compound 33 | 10 | 0/4 # | 30.0 ± 0.0 ** | 0.0 ± 0.0 * | 0.0 ± 0.0 * |
| | | Compound 25 | 10 | 0/4 # | 30.0 ± 0.0 ** | 0.0 ± 0.0 * | 0.0 ± 0.0 * |
| | | Compound 20 | 10 | 0/4 # | 30.0 ± 0.0 ** | 0.0 ± 0.0 * | 0.0 ± 0.0 * |
| | Emesis | Vehicle^{c} | — | 4/4 | 3.8 ± 1.7 | 12.3 ± 9.3 | 41.0 ± 14.0 |
| | | Compound 33 | 10 | 0/4 # | 30.0 ± 0.0 ** | 0.0 ± 0.0 * | 0.0 ± 0.0 ** |
| | | Compound 25 | 10 | 0/4 # | 30.0 ± 0.0 ** | 0.0 ± 0.0 * | 0.0 ± 0.0 ** |
| | | Compound 20 | 10 | 0/4 # | 30.0 ± 0.0 ** | 0.0 ± 0.0 * | 0.0 ± 0.0 ** |
| | Licking | Vehicle^{c} | — | 4/4 | 4.3 ± 2.1 | 10.5 ± 10.4 | 3.0 ± 0.8 |
| | | Compound 33 | 10 | 0/4 # | 30.0 ± 0.0 ** | 0.0 ± 0.0 | 0.0 ± 0.0 ** |
| | | Compound 25 | 10 | 0/4 # | 30.0 ± 0.0 ** | 0.0 ± 0.0 | 0.0 ± 0.0 ** |
| | | Compound 20 | 10 | 0/4 # | 30.0 ± 0.0 ** | 0.0 ± 0.0 | 0.0 ± 0.0 ** |
| | Fidget | Vehicle^{c} | — | 4/4 | 5.8 ± 3.1 | 9.8 ± 11.1 | 3.5 ± 1.7 |
| | | Compound 33 | 10 | 0/4 # | 30.0 ± 0.0 ** | 0.0 ± 0.0 | 0.0 ± 0.0 * |
| | | Compound 25 | 10 | 0/4 # | 30.0 ± 0.0 ** | 0.0 ± 0.0 | 0.0 ± 0.0 * |
| | | Compound 20 | 10 | 0/4 # | 30.0 ± 0.0 ** | 0.0 ± 0.0 | 0.0 ± 0.0 * |

Each value represents the mean ± S.D.
^{a}The latency of ferret that did not show emetic response was considered as 30 min.
^{b}The cumulative total duration of emetic response.
^{c}0.09 w/v % Polysorbate 80/10% DMSO/PBS 0.5 mL/kg, s.c.
#: Significant difference from vehicle treatment p < 0.05 (Fisher's exact test)
* and **: Significant difference from vehicle treatment p < 0.05 and p < 0.01, respectively (Aspin-Welch's t -test, One-side)

TABLE 10
Effect of Compound 33, Compound 25 and Compound 20 on morphine (0.6 mg/kg, s.c.)-induced emesis in ferrets

| | | | | | Retching | | | Vomiting | | | Emesis (Retching + |
|---|---|---|---|---|---|---|---|---|---|---|---|
| | Drugs | | | Body | | Dura- | | | Dura- | | Vomiting) |
| Morphine was | and | Animal | Animal | weight | Latency^{a} | tion^{b} | Fre- | Latency^{a} | tion^{b} | Fre- | Latency^{a} |
| administered | dose | No. | ID No. | (kg) | (min) | (min) | quency | (min) | (min) | quency | (min) |
| 4 h | Vehicle^{c} | 77 | 4401 | 1.50 | 6 | 8 | 54 | 6 | 8 | 6 | 6 |
| after | | 74 | 4402 | 1.54 | 4 | 26 | 29 | 8 | 3 | 2 | 4 |
| | | 62 | 4403 | 1.63 | 3 | 6 | 42 | 8 | 1 | 1 | 3 |
| | | 65 | 4404 | 1.54 | 2 | 9 | 27 | 3 | 8 | 3 | 2 |
| | | | Mean ± | 1.55 | 3.8 | 12.3 | 38.0 | 6.3 | 5.0 | 3.0 | 3.8 |
| | | | S.D. | 0.06 | 1.7 | 9.3 | 12.6 | 2.4 | 3.6 | 2.2 | 1.7 |
| | Compound 33 | 81 | 4501 | 1.63 | 30 | 0 | 0 | 30 | 0 | 0 | 30 |
| | 10 nmol/kg, s.c. | 93 | 4502 | 1.52 | 30 | 0 | 0 | 30 | 0 | 0 | 30 |
| | | 88 | 4503 | 1.82 | 30 | 0 | 0 | 30 | 0 | 0 | 30 |
| | | 69 | 4504 | 1.73 | 30 | 0 | 0 | 30 | 0 | 0 | 30 |
| | | | Mean ± | 1.68 | 30.0 | 0.0 | 0.0 | 30.0 | 0.0 | 0.0 | 30.0 |
| | | | S.D. | 0.13 | 0.0 | 0.0 | 0.0 | 0.0 | 0.0 | 0.0 | 0.0 |
| | Compound 25 | 61 | 4601 | 1.52 | 30 | 0 | 0 | 30 | 0 | 0 | 30 |
| | 10 nmol/kg, s.c. | 63 | 4602 | 1.50 | 30 | 0 | 0 | 30 | 0 | 0 | 30 |
| | | 99 | 4603 | 1.79 | 30 | 0 | 0 | 30 | 0 | 0 | 30 |
| | | 79 | 4604 | 1.48 | 30 | 0 | 0 | 30 | 0 | 0 | 30 |
| | | | Mean ± | 1.57 | 30.0 | 0.0 | 0.0 | 30.0 | 0.0 | 0.0 | 30.0 |
| | | | S.D. | 0.15 | 0.0 | 0.0 | 0.0 | 0.0 | 0.0 | 0.0 | 0.0 |
| | Compound 20 | 78 | 4701 | 1.46 | 30 | 0 | 0 | 30 | 0 | 0 | 30 |
| | 10 nmol/kg, s.c. | 94 | 4702 | 1.80 | 30 | 0 | 0 | 30 | 0 | 0 | 30 |
| | | 91 | 4703 | 1.73 | 30 | 0 | 0 | 30 | 0 | 0 | 30 |
| | | 95 | 4704 | 1.72 | 30 | 0 | 0 | 30 | 0 | 0 | 30 |
| | | | Mean ± | 1.68 | 30.0 | 0.0 | 0.0 | 30.0 | 0.0 | 0.0 | 30.0 |
| | | | S.D. | 0.15 | 0.0 | 0.0 | 0.0 | 0.0 | 0.0 | 0.0 | 0.0 |

| | | | | Emesis (Retching + | | | | | | | |
|---|---|---|---|---|---|---|---|---|---|---|---|
| | | | | Vomiting) | | Licking | | | Fidget | | |
| | Drugs | | | Dura- | | | Dura- | | | Dura- | |
| Morphine was | and | Animal | Animal | tion^{b} | Fre- | Latency^{a} | tion^{b} | Fre- | Latency^{a} | tion^{b} | Fre- |
| administered | dose | No. | ID No. | (min) | quency | (min) | (min) | quency | (min) | (min) | quency |
| 4 h | Vehicle^{c} | 77 | 4401 | 8 | 60 | 7 | 7 | 3 | 6 | 7 | 4 |
| after | | 74 | 4402 | 26 | 31 | 4 | 26 | 2 | 4 | 26 | 5 |
| | | 62 | 4403 | 6 | 43 | 4 | 5 | 4 | 3 | 5 | 4 |
| | | 65 | 4404 | 9 | 30 | 2 | 4 | 3 | 10 | 1 | 1 |
| | | | Mean ± | 12.3 | 41.0 | 4.3 | 10.5 | 3.0 | 5.8 | 9.8 | 3.5 |
| | | | S.D. | 9.3 | 14.0 | 2.1 | 10.4 | 0.8 | 3.1 | 11.1 | 1.7 |
| | Compound 33 | 81 | 4501 | 0 | 0 | 30 | 0 | 0 | 30 | 0 | 0 |
| | 10 nmol/kg, s.c. | 93 | 4502 | 0 | 0 | 30 | 0 | 0 | 30 | 0 | 0 |
| | | 88 | 4503 | 0 | 0 | 30 | 0 | 0 | 30 | 0 | 0 |
| | | 69 | 4504 | 0 | 0 | 30 | 0 | 0 | 30 | 0 | 0 |
| | | | Mean ± | 0.0 | 0.0 | 30.0 | 0.0 | 0.0 | 30.0 | 0.0 | 0.0 |
| | | | S.D. | 0.0 | 0.0 | 0.0 | 0.0 | 0.0 | 0.0 | 0.0 | 0.0 |
| | Compound 25 | 61 | 4601 | 0 | 0 | 30 | 0 | 0 | 30 | 0 | 0 |
| | 10 nmol/kg, s.c. | 63 | 4602 | 0 | 0 | 30 | 0 | 0 | 30 | 0 | 0 |
| | | 99 | 4603 | 0 | 0 | 30 | 0 | 0 | 30 | 0 | 0 |
| | | 79 | 4604 | 0 | 0 | 30 | 0 | 0 | 30 | 0 | 0 |
| | | | Mean ± | 0.0 | 0.0 | 30.0 | 0.0 | 0.0 | 30.0 | 0.0 | 0.0 |
| | | | S.D. | 0.0 | 0.0 | 0.0 | 0.0 | 0.0 | 0.0 | 0.0 | 0.0 |
| | Compound 20 | 78 | 4701 | 0 | 0 | 30 | 0 | 0 | 30 | 0 | 0 |
| | 10 nmol/kg, s.c. | 94 | 4702 | 0 | 0 | 30 | 0 | 0 | 30 | 0 | 0 |
| | | 91 | 4703 | 0 | 0 | 30 | 0 | 0 | 30 | 0 | 0 |
| | | 95 | 4704 | 0 | 0 | 30 | 0 | 0 | 30 | 0 | 0 |
| | | | Mean ± | 0.0 | 0.0 | 30.0 | 0.0 | 0.0 | 30.0 | 0.0 | 0.0 |
| | | | S.D. | 0.0 | 0.0 | 0.0 | 0.0 | 0.0 | 0.0 | 0.0 | 0.0 |

^{a}The latency of ferret that did not show emetic response was considered as 30 min.
^{b}The cumulative total duration of emetic response.
^{c}0.09 w/v % Polysorbate 80/10% DMSO/PBS 0.5 mL/kg, s.c.

Effects of Subcutaneously Administered Compound 15, Compound 16 and Compound 17 in Morphine-Induced Acute Emetic Model

30 nmol/kg of each compound dissolved with a vehicle (0.09 w/v % tween 80/10% DMSO/saline),respectively, to prepare test solutions. 0.5 mg/kg of the test solutions and the vehicle were subcutaneously administered to ferrets (4 in each group), respectively. At the time of each of 4 hours, or 120 hours after administration, 0.6 mg/kg of morphine was subcutaneously administered. Up to 360 minutes after morphine administration, the condition of the ferrets was monitored to record the frequencies and time points of abdominal contraction motions, vomiting behaviors, licking with the tongue, and fidgety behavior occurring.

Tables 11 and 12. Summary of the effects of Compound 15, Compound 16 and Compound 17 on morphine (0.6 mg/kg, s.c.)-induced emesis in ferrets (Summary Table 11, Individual Results Table 12)

TABLE 11
Summary of the effects of Compound 15, Compound 16 and Compound
17 on morphine (0.6 mg/kg, s.c.)-induced emesis in ferrets

| Morphine was | | | Dose | No. of animals | Latency^{a} | Duration^{b} | |
|---|---|---|---|---|---|---|---|
| administered | Signs | Drugs | (nmol/kg, s.c.) | (responder/used) | (min) | (min) | Frequency |
| 4 h | Retching | Vehicle^{c} | — | 4/4 | 4.8 ± 2.1 | 8.0 ± 1.4 | 28.5 ± 3.5 |
| after | | Compound 15 | 30 | 4/4 | 7.3 ± 2.9 | 4.5 ± 4.0 | 17.5 ± 6.6 * |
| | | Compound 16 | 30 | 0/4 # | 30.0 ± 0.0 ** | 0.0 ± 0.0 ** | 0.0 ± 0.0 ** |
| | | Compound 17 | 30 | 0/4 # | 30.0 ± 0.0 ** | 0.0 ± 0.0 ** | 0.0 ± 0.0 ** |
| | Vomiting | Vehicle^{c} | — | 4/4 | 5.5 ± 1.0 | 6.5 ± 1.7 | 4.8 ± 1.0 |
| | | Compound 15 | 30 | 3/4 | 12.0 ± 12.1 | 4.3 ± 4.3 | 3.0 ± 2.6 |
| | | Compound 16 | 30 | 0/4 # | 30.0 ± 0.0 ** | 0.0 ± 0.0 ** | 0.0 ± 0.0 ** |
| | | Compound 17 | 30 | 0/4 # | 30.0 ± 0.0 ** | 0.0 ± 0.0 ** | 0.0 ± 0.0 ** |
| | Emesis | Vehicle^{c} | — | 4/4 | 4.8 ± 2.1 | 8.0 ± 1.4 | 33.3 ± 3.8 |
| | | Compound 15 | 30 | 4/4 | 7.3 ± 2.9 | 4.5 ± 4.0 | 20.5 ± 8.3 * |
| | | Compound 16 | 30 | 0/4 # | 30.0 ± 0.0 ** | 0.0 ± 0.0 ** | 0.0 ± 0.0 ** |
| | | Compound 17 | 30 | 0/4 # | 30.0 ± 0.0 ** | 0.0 ± 0.0 ** | 0.0 ± 0.0 ** |
| | Licking | Vehicle^{c} | — | 4/4 | 8.3 ± 4.0 | 4.0 ± 3.8 | 3.3 ± 2.6 |
| | | Compound 15 | 30 | 3/4 | 12.3 ± 11.9 | 2.5 ± 3.7 | 1.5 ± 1.7 |
| | | Compound 16 | 30 | 0/4 # | 30.0 ± 0.0 ** | 0.0 ± 0.0 | 0.0 ± 0.0 * |
| | | Compound 17 | 30 | 0/4 # | 30.0 ± 0.0 ** | 0.0 ± 0.0 | 0.0 ± 0.0 * |
| | Fidget | Vehicle^{c} | — | 4/4 | 5.5 ± 2.6 | 6.3 ± 1.5 | 3.3 ± 0.5 |
| | | Compound 15 | 30 | 4/4 | 9.0 ± 3.2 | 1.0 ± 0.0 ** | 1.3 ± 0.5 ** |
| | | Compound 16 | 30 | 0/4 # | 30.0 ± 0.0 ** | 0.0 ± 0.0 ** | 0.0 ± 0.0 ** |
| | | Compound 17 | 30 | 0/4 # | 30.0 ± 0.0 ** | 0.0 ± 0.0 ** | 0.0 ± 0.0 ** |

Each value represents the mean ± S.D.
^{a}The latency of ferret that did not show emetic response was considered as 30 min.
^{b}The cumulative total duration of emetic response.
^{c}0.09 w/v % Polysorbate 80/10% DMSO/PBS 0.5 mL/kg, s.c.
#: Significant difference from vehicle treatment p < 0.05 (Fisher's exact test)
* and **: Significant difference from vehicle treatment p < 0.05 and p < 0.01, respectively (Student's t -test or Aspin-Welch's t -test, One-side)

TABLE 12
Effect of Compound 15, Compound 16 and Compound 17 on morphine (0.6 mg/kg, s.c.)-induced emesis in ferrets

| | | | | | | | | | | Emesis (Retching + |
|---|---|---|---|---|---|---|---|---|---|---|
| Drugs | | Aninal | Body | Retching | | | Vomiting | | | Vomiting) |
| and | Aninal | ID | weight | Latency^{a} | Duration^{b} | Fre- | Latency^{a} | Duration^{b} | Fre- | Latency^{a} |
| dose | No. | No. | (kg) | (min) | (min) | quency | (min) | (min) | quency | (min) |
| Vehicle^{c} | 2901 | 60 | 1.30 | 2 | 8 | 32 | 5 | 5 | 4 | 2 |
| | 2902 | 36 | 1.39 | 5 | 9 | 31 | 5 | 8 | 6 | 5 |
| | 2903 | 49 | 1.76 | 7 | 6 | 26 | 7 | 5 | 4 | 7 |
| | 2904 | 59 | 1.44 | 5 | 9 | 25 | 5 | 8 | 5 | 5 |
| | | Mean ± | 1.47 | 4.8 | 8.0 | 28.5 | 5.5 | 6.5 | 4.8 | 4.8 |
| | | S.D. | 0.20 | 2.1 | 1.4 | 3.5 | 1.0 | 1.7 | 1.0 | 2.1 |
| Compound 15 | 3001 | 39 | 1.46 | 5 | 8 | 21 | 5 | 8 | 6 | 5 |
| 30 nmol/kg, s.c. | 3002 | 51 | 1.55 | 11 | 1 | 9 | 30 | 0 | 0 | 11 |
| | 3003 | 55 | 1.40 | 5 | 8 | 24 | 5 | 8 | 2 | 5 |
| | 3004 | 53 | 1.46 | 8 | 1 | 16 | 8 | 1 | 4 | 8 |
| | | Mean ± | 1.47 | 7.3 | 4.5 | 17.5 | 12.0 | 4.3 | 3.0 | 7.3 |
| | | S.D. | 0.06 | 2.9 | 4.0 | 6.6 | 12.1 | 4.3 | 2.6 | 2.9 |
| Compound 16 | 3101 | 46 | 1.70 | 30 | 0 | 0 | 30 | 0 | 0 | 30 |
| 30 nmol/kg, s.c. | 3102 | 40 | 1.56 | 30 | 0 | 0 | 30 | 0 | 0 | 30 |
| | 3103 | 31 | 1.56 | 30 | 0 | 0 | 30 | 0 | 0 | 30 |
| | 3104 | 27 | 1.58 | 30 | 0 | 0 | 30 | 0 | 0 | 30 |
| | | Mean ± | 1.60 | 30.0 | 0.0 | 0.0 | 30.0 | 0.0 | 0.0 | 30.0 |
| | | S.D. | 0.07 | 0.0 | 0.0 | 0.0 | 0.0 | 0.0 | 0.0 | 0.0 |
| Compound 17 | 3201 | 56 | 1.26 | 30 | 0 | 0 | 30 | 0 | 0 | 30 |
| 30 nmol/kg, s.c. | 3202 | 41 | 1.54 | 30 | 0 | 0 | 30 | 0 | 0 | 30 |
| | 3203 | 44 | 1.24 | 30 | 0 | 0 | 30 | 0 | 0 | 30 |
| | 3204 | 54 | 1.50 | 30 | 0 | 0 | 30 | 0 | 0 | 30 |
| | | Mean ± | 1.39 | 30.0 | 0.0 | 0.0 | 30.0 | 0.0 | 0.0 | 30.0 |
| | | S.D. | 0.16 | 0.0 | 0.0 | 0.0 | 0.0 | 0.0 | 0.0 | 0.0 |

| | | | Emesis (Retching + | | | | | | | |
|---|---|---|---|---|---|---|---|---|---|---|
| Drugs | | Aninal | Vomiting) | | Licking | | | Fidget | | |
| and | Aninal | ID | Duration^{b} | Fre- | Latency^{a} | Duration^{b} | Fre- | Latency^{a} | Duration^{b} | Fre- |
| dose | No. | No. | (min) | quency | (min) | (min) | quency | (min) | (min) | quency |
| Vehicle^{c} | 2901 | 60 | 8 | 36 | 6 | 1 | 1 | 2 | 8 | 3 |
| | 2902 | 36 | 9 | 37 | 14 | 1 | 1 | 5 | 7 | 3 |
| | 2903 | 49 | 6 | 30 | 8 | 5 | 6 | 7 | 5 | 4 |
| | 2904 | 59 | 9 | 30 | 5 | 9 | 5 | 8 | 5 | 3 |
| | | Mean ± | 8.0 | 33.3 | 8.3 | 4.0 | 3.3 | 5.5 | 6.3 | 3.3 |
| | | S.D. | 1.4 | 3.8 | 4.0 | 3.8 | 2.6 | 2.6 | 1.5 | 0.5 |
| Compound 15 | 3001 | 39 | 8 | 27 | 6 | 1 | 1 | 12 | 1 | 1 |
| 30 nmol/kg, s.c. | 3002 | 51 | 1 | 9 | 30 | 0 | 0 | 11 | 1 | 1 |
| | 3003 | 55 | 8 | 26 | 5 | 8 | 4 | 5 | 1 | 2 |
| | 3004 | 53 | 1 | 20 | 8 | 1 | 1 | 8 | 1 | 1 |
| | | Mean ± | 4.5 | 20.5 | 12.3 | 2.5 | 1.5 | 9.0 | 1.0 | 1.3 |
| | | S.D. | 4.0 | 8.3 | 11.9 | 3.7 | 1.7 | 3.2 | 0.0 | 0.5 |
| Compound 16 | 3101 | 46 | 0 | 0 | 30 | 0 | 0 | 30 | 0 | 0 |
| 30 nmol/kg, s.c. | 3102 | 40 | 0 | 0 | 30 | 0 | 0 | 30 | 0 | 0 |
| | 3103 | 31 | 0 | 0 | 30 | 0 | 0 | 30 | 0 | 0 |
| | 3104 | 27 | 0 | 0 | 30 | 0 | 0 | 30 | 0 | 0 |
| | | Mean ± | 0.0 | 0.0 | 30.0 | 0.0 | 0.0 | 30.0 | 0.0 | 0.0 |
| | | S.D. | 0.0 | 0.0 | 0.0 | 0.0 | 0.0 | 0.0 | 0.0 | 0.0 |
| Compound 17 | 3201 | 56 | 0 | 0 | 30 | 0 | 0 | 30 | 0 | 0 |
| 30 nmol/kg, s.c. | 3202 | 41 | 0 | 0 | 30 | 0 | 0 | 30 | 0 | 0 |
| | 3203 | 44 | 0 | 0 | 30 | 0 | 0 | 30 | 0 | 0 |
| | 3204 | 54 | 0 | 0 | 30 | 0 | 0 | 30 | 0 | 0 |
| | | Mean ± | 0.0 | 0.0 | 30.0 | 0.0 | 0.0 | 30.0 | 0.0 | 0.0 |
| | | S.D. | 0.0 | 0.0 | 0.0 | 0.0 | 0.0 | 0.0 | 0.0 | 0.0 |

A summary of all results and experiments testing the various GIP receptor agonist peptides of the two experiments above are provided in summary form in Table 10.

TABLE 13
Summary of Anti-Emetic Activity of GIP receptor agonist
peptides on morphine-induced emesis in male ferrets

| Compound No. | SEQ ID NO: | MS [Calc] | Comment |
|---|---|---|---|
| 1 | 4 | 5037.6 | 100% inhibition |
| 2 | 5 | 5233.0 | partial inhibition |
| 3 | 6 | 5233.0 | partial inhibition |
| 4 | 7 | 3532.9 | 100% inhibition |
| 5 | 8 | 5410.1 | 100% inhibition |
| 9 | 12 | 4987.6 | 100% inhibition |
| 11 | 14 | 4277.8 | 100% inhibition |
| 12 | 15 | 4277.8 | partial inhibition |
| 15 | 18 | 4422.9 | partial inhibition |
| 16 | 19 | 5081.8 | 100% inhibition |
| 17 | 20 | 4276.7 | 100% inhibition |
| 20 | 23 | 4843.4 | 100% inhibition |
| 25 | 28 | 4278.8 | 100% inhibition |
| 33 | 36 | 4743.3 | 100% inhibition |

### Example 7 Effect of Subcutaneously Injected Compound 17 and 19 in Cisplatin-Induced Acute Vomiting Model

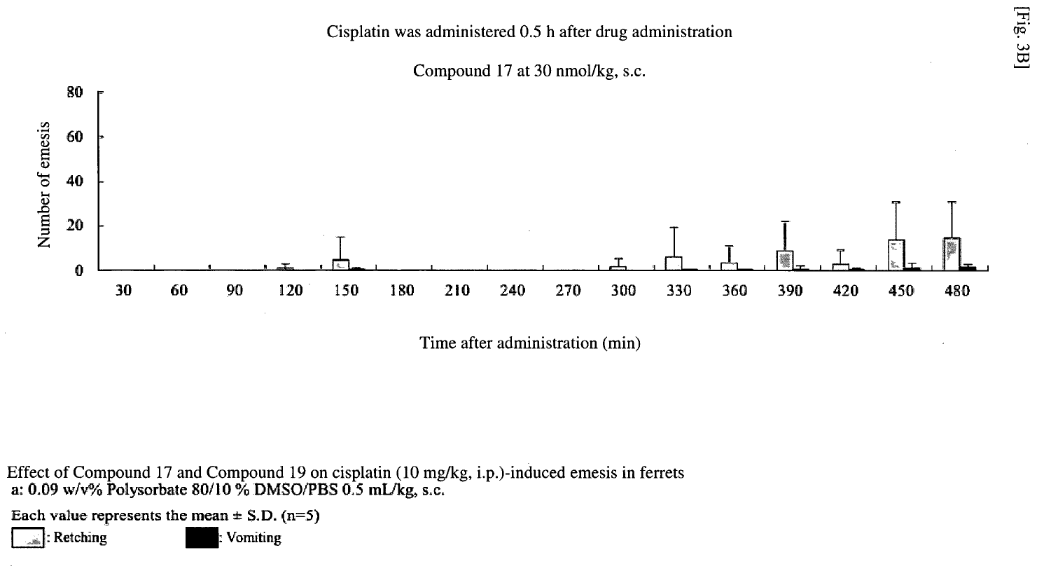

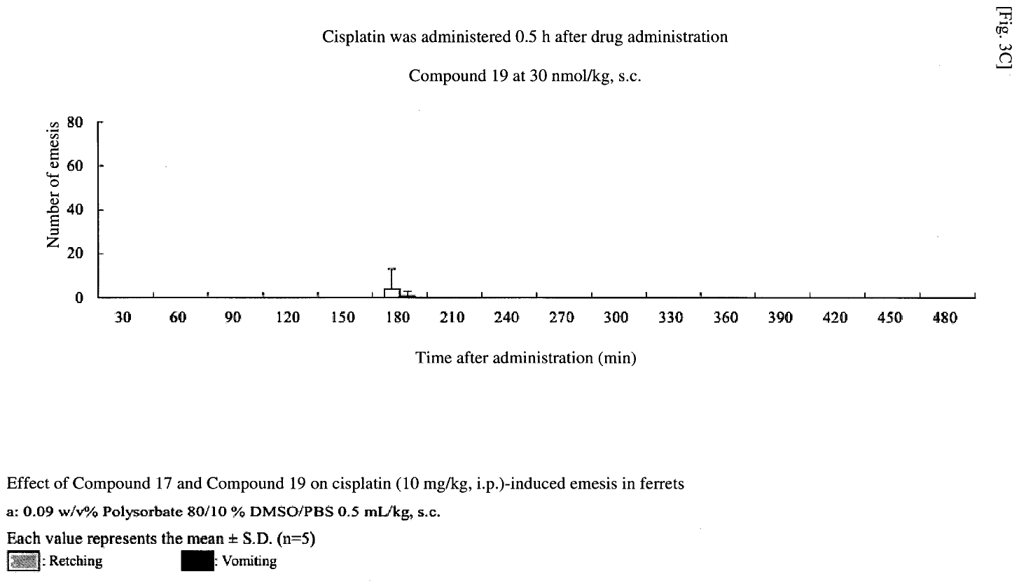

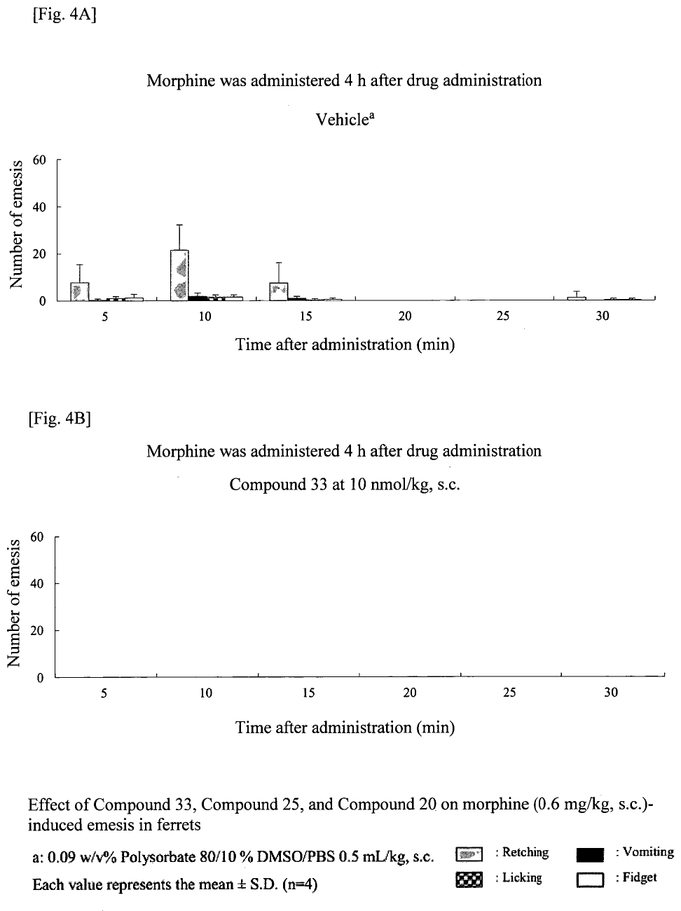

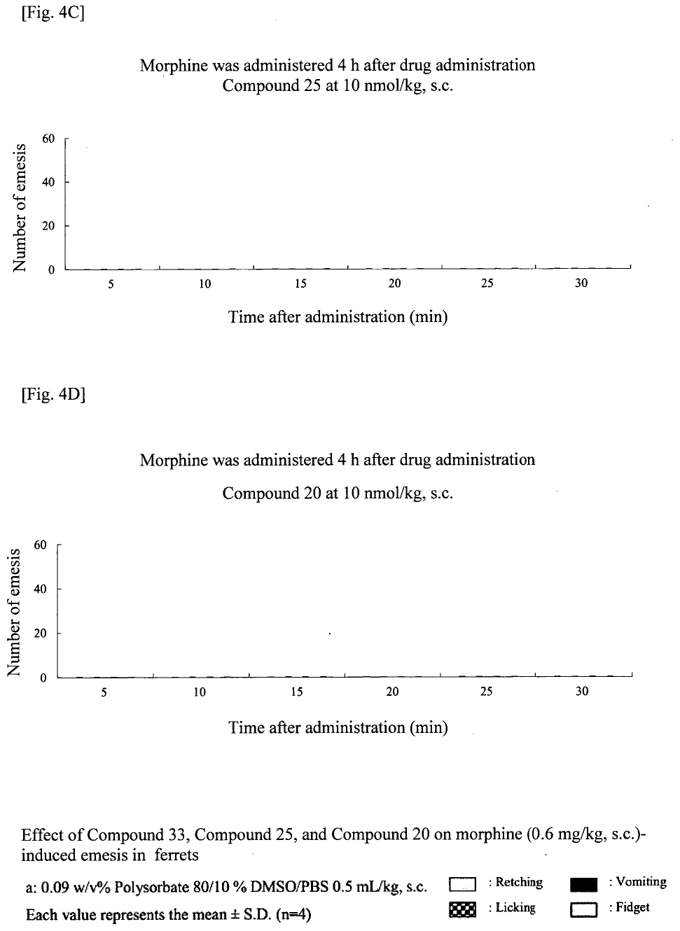

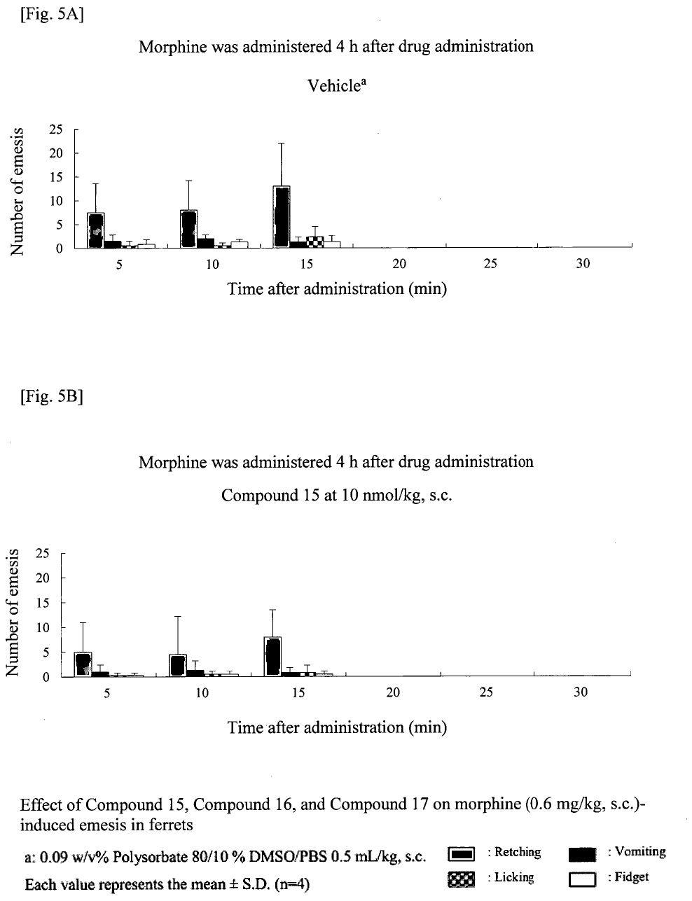

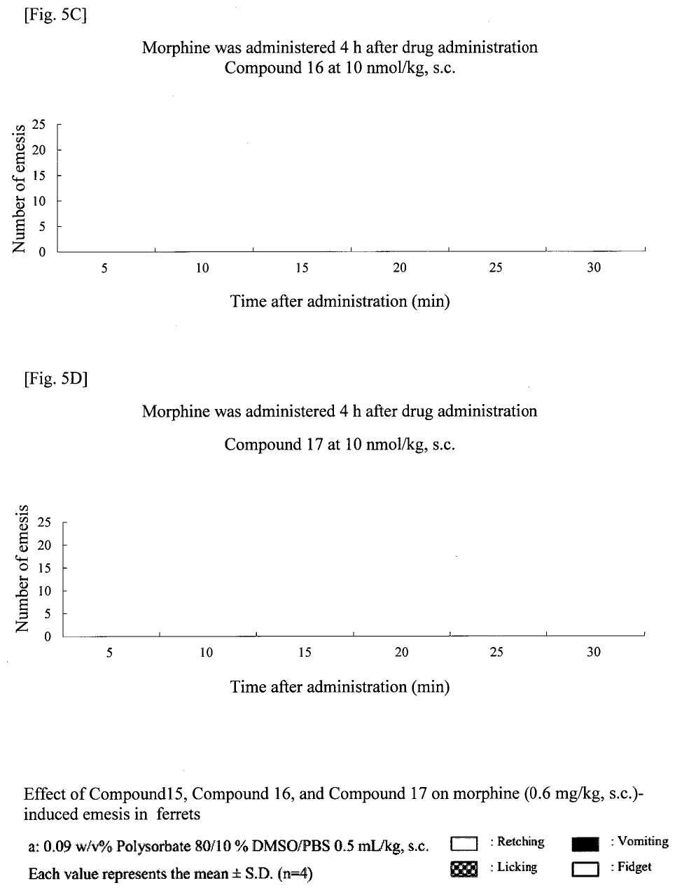

Cisplatin (10 mg/kg i.p.) was administered to male ferrets, n=5) to induce acute vomiting. A vehicle (0.09 w/v % Polysorbate 80/10% DMSO/PBS, was administered subcutaneously.), Compound 17 (30 nmol/kg), and Compound 19 (30 nmol/kg) were subcutaneously injected at 0.5 hour before cisplatin administration. Emetic events (dry vomiting plus actual vomiting) were evaluated using the above ferrets. The frequency, latency and duration of emesis were monitored over a period of 8 hours after cisplatin administration. The condition of the ferrets was monitored to record the number of animals that did not vomit, the number of emetic episodes, the latency period in minutes to observe the emetic episodes, the duration of the observed emesis if any. See results in Table 14 and FIGS. 3A-3C.

Results shown in Table 14 and FIGS. 3A-3C, illustrate the anti-emetic effects of Compound 17, and Compound 19. Compound 19 showed a more durable response than Compound 17. Both Compounds 17 and 19 were statistically significant in various tested anti-emetic properties compared to the vehicle control.

Tables 14 and 15. Effect of Compounds 17 and Compound 19 on cisplatin (10 mg/kg, i.p.)-induced emesis in ferrets (Summary Table 14, Individual Results Table 15).

TABLE 14
Effect of Compounds 17 and Compound 19 on cisplatin (10 mg/kg, i.p.)-induced emesis in ferrets

| Cisplatin was | | | Dose | No. of animals | Latency^{a} | Duration^{b} | |
|---|---|---|---|---|---|---|---|
| administered | Signs | Drugs | (nmol/kg, s.c.) | (responder/used) | (min) | (min) | Frequency |
| 0.5 h | Retching | Vehicle^{c} | — | 5/5 | 124.2 ± 79.6 | 262.8 ± 152.4 | 145.2 ± 102.6 |
| after | | Compound 17 | 30 | 5/5 | 360.2 ± 152.1 ** | 97.4 ± 163.0 | 56.4 ± 61.8 |
| | | Compound 19 | 30 | 1/5 # | 417.4 ± 140.0 ** | 0.2 ± 0.4 ** | 4.0 ± 8.9 * |
| | Vomiting | Vehicle^{c} | — | 5/5 | 124.8 ± 79.4 | 262.6 ± 152.1 | 20.4 ± 12.3 |
| | | Compound 17 | 30 | 4/5 | 371.8 ± 148.0 ** | 92.2 ± 153.4 | 4.4 ± 3.4 * |
| | | Compound 19 | 30 | 1/5 # | 417.4 ± 140.0 ** | 0.2 ± 0.4 ** | 0.8 ± 1.8 * |
| | Emesis | Vehicle^{c} | — | 5/5 | 124.2 ± 79.6 | 263.2 ± 152.7 | 165.6 ± 114.4 |
| | | Compound 17 | 30 | 5/5 | 360.2 ± 152.1 ** | 97.4 ± 163.0 | 60.8 ± 64.8 |
| | | Compound 19 | 30 | 1/5 # | 417.4 ± 140.0 ** | 0.2 ± 0.4 ** | 4.8 ± 10.7 * |

Each value represents the mean ± S.D.
^{a}The latency of ferret that did not show emetic response was considered as 480 min.
^{b}The cumulative total duration of emetic response.
^{c}0.09 w/v % Polysorbate 80/10% DMSO/PBS 0.5 mL/kg, s.c.
#: Significant difference from vehicle treatment p < 0.05 (Fisher's exact test)
* and **: Significant difference from vehicle treatment p < 0.05 and p < 0.01, respectively (Student's t -test or Aspin-Welch's t -test, One-side)

TABLE 15
Effect of Compounds 17 and Compound 19 on cisplatin (10 mg/kg, i.p.)-induced emesis in ferrets

| | | | | | | | | | | Emesis (Retching + | | |
|---|---|---|---|---|---|---|---|---|---|---|---|---|
| | | | | Retching | | | Vomiting | | | Vomiting) | | |
| | Drugs | | Animal | | Dura- | | | Dura- | | | Dura- | |
| Cisplatin was | and | Animal | ID | Latency^{a} | tion^{b} | Fre- | Latency^{a} | tion^{b} | Fre- | Latency^{a} | tion^{b} | Fre- |
| administered | dose | No. | No. | (min) | (min) | quency | (min) | (min) | quency | (min) | (min) | quency |
| 0.5 h | Vehicle^{c} | 3301 | 18 | 265 | 1 | 3 | 265 | 1 | 2 | 265 | 1 | 5 |
| after | | 3302 | 21 | 80 | 339 | 254 | 80 | 339 | 31 | 80 | 339 | 285 |
| | | 3303 | 10 | 93 | 379 | 234 | 96 | 376 | 32 | 93 | 379 | 266 |
| | | 3304 | 19 | 76 | 261 | 135 | 76 | 261 | 16 | 76 | 261 | 151 |
| | | 3305 | 12 | 107 | 334 | 100 | 107 | 336 | 21 | 107 | 336 | 121 |
| | | | Mean ± | 124.2 | 262.8 | 145.2 | 124.8 | 262.6 | 20.4 | 124.2 | 263.2 | 165.6 |
| | | | S.D. | 79.6 | 152.4 | 102.6 | 79.4 | 152.1 | 12.3 | 79.6 | 152.7 | 114.4 |
| | Compound 17 | 3401 | 9 | 479 | 1 | 10 | 480 | 1 | 2 | 479 | 1 | 12 |
| | 30 nmol/kg, s.c. | 3402 | 14 | 424 | 3 | 33 | 425 | 2 | 5 | 424 | 3 | 38 |
| | | 3403 | 22 | 349 | 104 | 81 | 349 | 103 | 7 | 349 | 104 | 88 |
| | | 3404 | 5 | 102 | 378 | 153 | 125 | 355 | 8 | 102 | 378 | 161 |
| | | 3405 | 16 | 447 | 1 | 5 | 480 | 0 | 0 | 447 | 1 | 5 |
| | | | Mean ± | 360.2 | 97.4 | 56.4 | 371.8 | 92.2 | 4.4 | 360.2 | 97.4 | 60.8 |
| | | | S.D. | 152.1 | 163.0 | 61.8 | 148.0 | 153.4 | 3.4 | 152.1 | 163.0 | 64.8 |
| | Compound 19 | 3501 | 20 | 480 | 0 | 0 | 480 | 0 | 0 | 480 | 0 | 0 |
| | 30 nmol/kg, s.c. | 3502 | 13 | 480 | 0 | 0 | 480 | 0 | 0 | 480 | 0 | 0 |
| | | 3503 | 15 | 480 | 0 | 0 | 480 | 0 | 0 | 480 | 0 | 0 |
| | | 3504 | 25 | 167 | 1 | 20 | 167 | 1 | 4 | 167 | 1 | 24 |
| | | 3505 | 29 | 480 | 0 | 0 | 480 | 0 | 0 | 480 | 0 | 0 |
| | | | Mean ± | 417.4 | 0.2 | 4.0 | 417.4 | 0.2 | 0.8 | 417.4 | 0.2 | 4.8 |
| | | | S.D. | 140.0 | 0.4 | 8.9 | 140.0 | 0.4 | 1.8 | 140.0 | 0.4 | 10.7 |

^{a}The latency of ferret that did not show emetic response was considered as 480 min.
^{b}The cumulative total duration of emetic response.
^{c}0.09 w/v % Polysorbate 80/10% DMSO/PBS 0.5 mL/kg, s.c.

### Example 8 PYY-1119-Induced Vomiting in Dogs

Compound 33, Compound 22, and Compound 26 or vehicle (0.09% [w/v] Tween 80/10% DMSO/PBS) was administered subcutaneously (sc) to female beagle dogs (10 months old), followed by subcutaneous (sc) injections with Y2R agonist ((4-imidazolecarbonyl-Ser-D-Hyp-Iva-Pya(4)-Cha-Leu(Me)-Asn-Lys-Aib-Thr-Arg-Gln-Arg-Cha-NH₂), 10 μg/kg), 10 μg/kg) at 8 hours postdose. Emetic episodes were counted for 2 hours after administration (by blinded analysis).

Result are shown in Table 16.

TABLE 16

| | | | | No. of | No. of | Latency | |
|---|---|---|---|---|---|---|---|
| Day | | | | animals that | emetic | period of | Duration |
| [after | Test | Dose | | did not | episodes | emesis | of emesis |
| dosing] | article | (nmol/kg) | N | vomit | [% Inhibition ^{a)}] | (min) | (min) |
| 1 | Vehicle | 0 | 4 | 0 | 7.3 ± 2.6 | 5.8 ± 1.0 | 19.5 ± 8.4 |
| [8 hr] | Compound 33 | 30 | 4 | 2 | 0.8 ± 1.0^{#} | 66.8 ± 61.5 | 0.3 ± 0.5^{#} |
| | | | | | [89.7%] | | |
| | Compound 22 | 30 | 4 | 2 | 1.3 ± 1.5** | 66.8 ± 61.5 | 0.5 ± 0.6^{#} |
| | | | | | [82.8%] | | |
| | Compound 26 | 30 | 4 | 3 | 0.3 ± 0.5^{#} | 92.3 ± 55.5 | 0.0 ± 0.0^{#} |
| | | | | | [96.6%] | | |

Significantly different from the vehicle group;
*: p ≤ 0.05,
**: p ≤ 0.01 (Student’s t test),
^{#}p ≤ 0.05,
^{##}: p ≤ 0.01 (Aspin & Welch t test), Data is expressed as mean ± S.D.,
ND: Not determined
^{a)} Decreased ratio of the mean emetic episodes compared to those in the vehicle group

Effect of Compound 19 on Y2R Agonist-Induced Emesis in Dogs

Compound 19 or vehicle (0.09% [w/v] Tween 80/10% DMSO/PBS) was administered subcutaneously (sc) to female beagle dogs (16 to 26 months old), followed by sc injections with Y2R agonist (4-imidazolecarbonyl-Ser-D-Hyp-Iva-Pya(4)-Cha-Leu(Me)-Asn-Lys-Aib-Thr-Arg-Gln-Arg-Cha-NH₂, 10 μg/kg) at 1 hour postdose. Emetic episodes were counted for 2 hours after Y2R agonist administration (by blinded analysis).

Results are shown in Table 17

TABLE 17
Effect of Compound 19 on Y2R agonist-induced emesis in dogs

| | | | | No. of | No. of | Latency | |
|---|---|---|---|---|---|---|---|
| Day | | | | animals that | emetic | period of | Duration |
| [after | Test | Dose | | did not | episodes | emesis | of emesis |
| dosing] | article | (nmol/kg) | N | vomit | [% Inhibition ^{a)}] | (min) | (min) |
| 1 | Vehicle | 0 | 4 | 0 | 7.3 ± 3.0 | 5.3 ± 1.0 | 32.5 ± 34.8 |
| [1 hr] | Compound 19 | 30 | 4 | 2 | 1.0 ± 1.2 | 67.5 ± 60.7 | 0.5 ± 0.6 |
| | | | | | [86.2%] | | |

Significantly different from the vehicle group;
*: p ≤ 0.05 (Student's t test),
^{#}: p ≤ 0.05 (Aspin & Welch t test), Data is expressed as mean ± S.D.,
ND: Not determined
^{a)}Decreased ratio of the mean emetic episodes compared to those in the vehicle group

As shown in Tables 16 and 17, the Compounds 33, 22, 26, and 19 were shown to have significant anti-emetic effects in animal models of emesis (dogs). Specifically, the compounds tested inhibited the number of emetic episodes by at least 80%, ranging from 83 to 97%.

### Formulation Example 1

| | | | |
|---|---|---|---|
| (1) | Compound 5 | 10.0 | mg |
| (2) | Lactose | 70.0 | mg |
| (3) | Cornstarch | 50.0 | mg |
| (4) | Soluble starch | 7.0 | mg |
| (5) | Magnesium stearate | 3.0 | mg |

Compound 5 (10.0 mg) and magnesium stearate (3.0 mg) are granulated with an aqueous soluble starch solution (0.07 mL) (7.0 mg as soluble starch), dried and mixed with lactose (70.0 mg) and cornstarch (50.0 mg). The mixture is compressed to give a tablet.

### Formulation Example 2

| | | | |
|---|---|---|---|
| (1) | Compound 11 | 5.0 | mg |
| (2) | Sodium chloride | 20.0 | mg |
| (3) | Distilled water to total amount | 2 | mL |

Compound 11 (5.0 mg) and sodium chloride (20.0 mg) are dissolved in distilled water, and water is added to a total amount of 2.0 ml. The solution is filtered, and filled in a 2 ml ampoule under aseptic conditions. The ampoule is sterilized and tightly sealed to give a solution for injection.

### INDUSTRIAL APPLICABILITY

The GIP receptor agonist peptides of the present disclosure have superior GIP receptor selective agonist activity and are useful as a drug for the prophylaxis or treatment of emesis and conditions caused by associated with GIP receptor activity, for example, emesis and diseases associated with vomiting or nausea and the like. In one embodiment, the GIP receptor agonist peptides are useful as a drug or medicament, or for use in the prophylaxis or treatment of emesis and conditions caused by associated with GIP receptor activity, for example cyclic vomiting syndrome, and nausea and/or vomiting associated with administration of a chemotherapeutic or anti-cancer agent as illustrated herein.

All the publications, patents, and the patent applications cited herein are incorporated herein by reference in their entireties.

### Free Text for Sequence Listing

SEQ ID NO: 1: Natural human GIP (1-42 peptide)
SEQ ID NO: 2: Natural human GIP (1-153; Signal peptide: 1-21; Propeptide: 22-50; Peptide: 52-93; Propeptide: 95-153.) mvatktfall llslflavgl gekkeghfsa lpslpvgsha kvsspqprgp ryaegtfisd ysiamdkihq qdfvnwllaq kgkkndwkhn itqrearale lasqanrkee eavepqsspa
knpsdedllr dlliqellac lldqtnlcrl rsr (153 a.a. Accession No. NP_004114.1 (SEQ ID NO: 2) SEQ ID NO: 3: mRNA sequence of natural human GIP of SEQ ID NO: 2 (NCBI Accession No. 004123.3)
SEQ ID NOs: 4 to 37: GIP selective agonist peptides (Compounds 1-34).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

1. A method for treating emesis in a subject, the method comprising administering a therapeutically effective amount of a GIP receptor agonist peptide, or a salt thereof, with the proviso that the GIP receptor agonist peptide does not have an amino acid sequence as set forth in SEQ ID NO: 1.

2. A method for treating emesis in a subject, the method comprising administering a therapeutically effective amount of a GIP receptor agonist peptide, or a salt thereof, with the proviso that the peptide does not have an amino acid sequence as set forth in any one of SEQ ID NOs: 4 to 569 disclosed in PCT Application No. PCT/JP2018/013540.

3. A method for treating emesis in a subject, the method comprising administering a therapeutically effective amount of a GIP receptor agonist peptide, or a salt thereof, wherein the GIP receptor agonist peptide has at least 80% sequence identity to amino acid sequence of the native human GIP peptide having an amino acid sequence as set forth in SEQ ID NO: 1, with the proviso that the peptide does not have an amino sequence as set forth in SEQ ID NO: 1.

4. A method for treating emesis in a subject, the method comprising administering a therapeutically effective amount of a GIP receptor agonist peptide having 29 or 30 amino acids, or a salt thereof.

5. A method for treating emesis in a subject, the method comprising administering a therapeutically effective amount of a GIP receptor agonist peptide, or a salt thereof, wherein the GIP receptor agonist peptide has at least 80%, at least 85%, at least 90%, at least 95%, at least 96%, or at least 97%, sequence identity to amino acid sequence of the native human GIP peptide having an amino acid sequence as set forth in SEQ ID NO: 1, with the proviso that the peptide does not have an amino sequence as set forth in SEQ ID NO: 1.

6. A method for treating emesis in a subject, the method comprising administering a therapeutically effective amount of a GIP receptor agonist peptide having 29 or 30 amino acids, or a salt thereof, wherein the GIP receptor agonist peptide has at least 80% sequence identity to amino acid sequence 1-29 or 1-30 of the native human GIP peptide having an amino acid sequence as set forth in SEQ ID NO: 1.

7. A method for treating emesis in a subject, the method comprising administering a therapeutically effective amount of a GIP receptor agonist peptide having 29 or 30 amino acids, or a salt thereof, wherein the GIP receptor agonist peptide has at least 80% sequence identity to amino acid sequence 1-29 or 1-30 of the native human GIP peptide having an amino acid sequence as set forth in SEQ ID NO: 1, with the proviso that the GIP receptor agonist peptide does not have an amino acid sequence as set forth in any 29 or 30 amino acid peptide provided in SEQ ID NOs: 4 to 569 disclosed in PCT Application No. PCT/JP2018/013540.

8. A method for treating emesis in a subject, the method comprising administering a therapeutically effective amount of a GIP receptor agonist peptide, or a salt thereof, said peptide having the formula (I): P′-A1-A2-A3-A4-A5-A6-A7-A8-A9-A10-A11-A12-A13-A14-A15-A16-A17-A 18-A19-A20-A21-A22-A23-A24-A25-A26-A27-A28-A29-A30-A31-A32-A33-A34-A35-A36-A37-A38-A39-A40-A41-A42-A43-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or
is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, or des-amino-Phe, or des-amino-Tyr;
A2 represents Ala, Aib, D-Ala, Gly, Ser, or Sar;
A3 represents Glu or Pro;
A4 represents Gly, or Ser;
A5 represents Thr, D-Iva, Glu, Iva, or Ser;
A6 represents Phe, Iva, Val, Ala, Aib, Cha, or α-methyl-Leu;
A7 represents Ile, Lys, Ala, Aib, Cha, D-Leu, Ile, Thr, Arg, or Val;
A8 represents Ser, Ala, ψ, or Aib;
A9 represents Asp, Leu, ψ, Phe, Glu, or Gln;
A10 represents Tyr, Leu, Ser, Cha, or ψ;
A11 represents Ser, Aib, A5c, A6c, D-Iva, or Iva;
A12 represents Ile, Lys, Glu, Asp, Ala, Aib, Lys-Ac, Ser, α-methyl-Phe, or ψ;
A13 represents Ala, Aib, Tyr, D-Iva, ψ, Gln, Leu, Glu, or Iva;
A14 represents Met, Nle, α-methyl-Leu, Leu, or ψ;
A15 represents Asp, Glu, Lys, Ser, Tyr, ψ, or Asn;
A16 represents Lys, Ala, Ser, Glu, Arg, Aib, Lys-Ac, or ψ;
A17 represents Ile, Lys, Arg, Aib, Gln, Glu, Lys-Ac, or ψ;
A18 represents His, Arg, Ala, Aib, D-Iva, Phe, Iva, Leu, Ser, Trp, or ψ;
A19 represents Gln, Lys, Glu, Ala, Val, Ser, Aib, Arg, or ψ;
A20 represents Gln, Lys, Ala, His, Arg, Aib, Asp, Gly, or ψ;
A21 represents Asp, Leu, Asn, Asp, Glu, Ala, Leu, Ser, Aib, or ψ;
A22 represents Phe, α-methyl-Phe, Naphthyl-Ala, Asn, Ala, Trp, or ψ;
A23 represents Val, Ile, or ψ;
A24 represents Asn, Asp, Glu, Ala, Aib, Gln, Glu, Lys, Lys-Ac, Leu, Nle, Arg, Ser, or ψ;
A25 represents Trp, Tyr, Glu, Phe, Arg, α-methyl-Phe, or ψ;
A26 represents Leu, Aib, Iva, Leu, Nle, or ψ;
A27 represents Leu, Glu, Ser, Lys, Val, Ile, Nle, or ψ;
A28 represents Ala, Ser, Arg, Leu, Met, Lys, Lys-Ac, Aib, or ψ;
A29 represents Gln, Gly, Ala, Thr, Ser, Lys, Aib, ψ, or deletion;
A30 represents Lys, Arg, Gly, Pro, Glu, Lys-Ac, ψ, or deletion;
A31 represents Phe, Pro, Gly, ψ, or deletion;
A32 represents Lys, Ser, Gly, ψ, or deletion;
A33 represents Lys, Ser, Gly, Ile, Ser, ψ, or deletion;
A34 represents Asn, Ala, Gly, Gln, ψ, or deletion;
A35 represents Asp, Ala, Ser, Pro, Glu, ψ, or deletion;
A36 represents Trp, Pro, Gly, ψ, or deletion;
A37 represents Lys, Pro, Gly, ψ, or deletion;
A38 represents His, Pro, Gly, Ser, ψ, or deletion;
A39 represents Asn, Ser, Gly, Asn, Lys, Gln, ψ, or deletion;
A40 represents Ile, Arg, Glu, Lys, Ser, Lys-Ac, Arg, ψ, or deletion;
A41 represents Ile, Thr, Gly, ψ, or deletion;
A42 represents Gln, Gly, ψ, or deletion;
A43 represents ψ, or deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

9. A method for treating emesis in a subject, the method comprising administering a therapeutically effective amount of a GIP receptor agonist peptide, or a salt thereof, said peptide having the formula (II): P¹-A1-A2-A3-Gly-Thr-A6-A7-Ser-A9-A10-A11-A12-A13-A14-A15-A16-A17-A18-A19-A20-A21-A22-A23-A24-Trp-Leu-A27-A28-A29-A30-A31-A32-A33-A34-A35-A36-A37-A38-A39-A40-A41-A42-A43-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent;
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, des-amino-Phe, or des-amino-Tyr;
A2 represents Ala, Aib, D-Ala, Gly, Ser, or Sar;
A3 represents Glu, or Pro;
A6 represents Phe, Iva, or Val;
A7 represents Ile, Lys, Thr, or Val;
A9 represents Asp, Leu, or Phe;
A10 represents Tyr, or ψ;
A11 represents Ser, A5c, Leu, Aib, or Cha;
A12 represents Ile, Lys, Glu, Asp, or ψ;
A13 represents Ala, Aib, Tyr, D-Iva, Gln, Leu, or Glu;
A14 represents Met, Nle, Leu, or ψ;
A15 represents Asp, Glu, Lys, Ser, or Tyr;
A16 represents Lys, Ala, Ser, Glu, Arg, or ψ;
All represents Ile, Lys, Arg, Aib, Gln, Ile, Glu, or ψ;
A18 represents His, Ala, or ψ;
A19 represents Gln, Lys, Glu, Ala, Val, Ser, or ψ;
A20 represents Gln, Lys, Ala, His, Arg, Aib, or ψ;
A21 represents Asp, Leu, Asn, Glu, Ala, Leu, Ser, or ψ;
A22 represents Phe, or ψ;
A23 represents Val, Ile, or ψ;
A24 represents Asn, Asp, Glu, Ala, Gln, Arg, Asn, Asp, Lys, Lys-Ac, or ψ;
A25 represents Trp, or ψ;
A26 represents Leu, Aib, Iva, or ψ;
A27 represents Leu, Glu, Ser, Lys, Val, Ile, or ψ;
A28 represents Ala, Ser, Arg, Leu, Met, Lys, Lys-Ac, or ψ;
A29 represents Gln, Gly, Ala, Thr, Ser, Lys, Aib, ψ, or deletion;
A30 represents Lys, Arg, Gly, Pro, ψ, or deletion;
A31 represents Phe, Pro, Gly, ψ, or deletion;
A32 represents Lys, Ser, Gly, ψ, or deletion;
A33 represents Lys, Ser, Gly, ψ, or deletion;
A34 represents Lys, Gly, Ala, Gln, ψ, or deletion;
A35 represents Asp, Ala, Ser, Pro, Glu, ψ, or deletion;
A36 represents Trp, Pro, Gly, ψ, or deletion;
A37 represents Lys, Pro, Gly, ψ, or deletion;
A38 represents His, Pro, Ser, Gly, ψ, or deletion;
A39 represents Asn, Lys, Gly, Gln, Ser, ψ, or deletion;
A40 represents Ile, Arg, Lys, Ser, ψ, or deletion;
A41 represents Ile, Thr, ψ, or deletion;
A42 represents Gln, Gly, ψ, or deletion;
A43 represents ψ, or deletion;
wherein ψ is a residue independently selected from Lys, Arg, Orn, and Cys and wherein the side chain of said residue is substituted, and with the proviso that the GIP receptor agonist peptide is not a peptide having an amino acid sequence of SEQ ID NOs: 4 to 569 disclosed in PCT Application No. PCT/JP2018/013540, and wherein, one or two amino acids selected from A8 to A42 optionally represent Lys(R), and R represents a substituent group, or a salt thereof.

10. A method for treating emesis in a subject, the method comprising administering a therapeutically effective amount of a GIP receptor agonist peptide, or a salt thereof, said peptide having at least 80% sequence identity to a peptide having the formula (III): P¹-A1-A2-A3-Gly-Thr-Phe-Ile-Ser-Asp-Tyr-A11-Ile-A13-A14-A15-A16-A17-His-Gln-A20-Asp-Phe-Val-A24-Trp-Leu-A27-A28-A29-A30-A31-A32-A33-A34-A35-A36-A37-A38-A39-A40-A41-A42-A43-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent;
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, di-Br-Tyr;
A2 represents Ala, or Aib;
A3 represents Glu, or Pro;
A11 represents Ser, A5c, or A6c;
A13 represents Ala, or Aib;
A14 represents Met, Leu or Nle;
A15 represents Asp or Glu;
A16 represents Lys, Ala, or Lys(R);
A17 represents Ile, or Lys(R);
A20 represents Gln, or Lys(R);
A24 represents Asn, or Asp;
A27 represents Leu or Lys(R);
A28 represents Ala or Lys(R);
A29 represents Gln or Lys(R);
A30 represents Lys, Pro, or Lys(R);
A31 represents Phe, Pro, or deletion;
A32 represents Lys, Ser, or deletion;
A33 represents Lys, Ser, or deletion;
A34 represents Asn, Ala, Gly, or deletion;
A35 represents Asp, Pro, Ala, or deletion;
A36 represents Trp, Pro, or deletion;
A37 represents Lys, Pro, Lys(R), or deletion;
A38 represents His, Pro, Ser, or deletion;
A39 represents Asn, Ser, or deletion;
A40 represents Ile, Lys(R), or deletion;
A41 represents Ile, Thr, or deletion;
A42 represents Gln, or deletion;
A43 represents ψ, or deletion;
wherein Lys(R) is a Lys residue and (R) represents a substitutent group, or salt thereof, with the proviso that the GIP receptor agonist peptide is not a peptide having an amino acid sequence of SEQ ID NOs: 4 to 569 disclosed in PCT Application No. PCT/JP2018/013540, and wherein, one or two amino acids selected from A8 to A42 optionally represent Lys(R), and the (R) in Lys(R) represents a substituent group, or a salt thereof.

11. The method of any one of claims 1-10, wherein the GIP receptor agonist peptide has at least 80%, or at least 85%, or at least 90%, or at least 95%, or at least 96%, or at least 97%, or at least 98%, or at least 99%, or at least 100% sequence identity to a GIP receptor agonist peptide having an amino acid sequence of SEQ ID NOs: 4-37.

12. A method for treating emesis in a subject, the method comprising administering a therapeutically effective amount of a GIP receptor agonist peptide, or a salt thereof, said peptide having at least 80% sequence identity to a peptide having the formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

13. A method for treating emesis in a subject, the method consisting of administering a therapeutically effective dose of a monotherapy, the monotherapy consisting essentially of a GIP receptor agonist peptide, or a salt thereof, said peptide having at least 80% sequence identity to a peptide having the formula (IV): P¹-A1-A2-A3-Gly-A5-A6-A7-A8-A9-A 10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

14. A method for treating emesis in a subject, the method consisting of administering a therapeutically effective dose of a monotherapy, the monotherapy consisting essentially of a GIP receptor agonist peptide, or a salt thereof, said peptide having at least 80% sequence identity to a peptide having the formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Ser, Pro or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln or a deletion;
with the proviso that if A14 is Leu or Lys(R), then the GIP receptor agonist peptide is at least 40 amino acids in length, and A40 is not Arg, Lys or Glu, and wherein any one or two amino acids selected from A8 to A42 optionally represent Lys(R), and the (R) in Lys(R) represents a substituent group.

15. A method for treating emesis in a subject, the method comprising administering a therapeutically effective amount of a GIP receptor agonist peptide, or a salt thereof, having an amino acid sequence of formula (V): P¹-Tyr-A2-A3-Gly-Thr-Phr-A7-Ser-Asp-A10-Ser-A12-Ala-A14-A15-A16-A 17-A18-A 19-A20-A21-Phe-A23-A24-A25-Leu-A27-A28-A29-A30-A31-A32-A33-A34-A35-A36-A37-A38-A39-A40-A41-A42-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib, Ala, Gly, Ser, D-Ala, or N-methyl-Ser;
A3 represents Glu, Asp, or Gln;
A7 represents Ile, or Ser, Thr;
A10 represents Tyr, Leu, or Ser;
A12 represents Ile, Lys, or ψ;
A14 represents Ile, Leu, or Met;
A15 represents Asp, or Glu;
A16 represents Lys, Ser, Glu or w;
A17 represents Ile, Lys, Gln, or ψ;
A18 represents Ala, Arg, or His;
A19 represents Gln, Ala, Lys, or Glu;
A20 represents Gln, Arg, or Lys, His or Ala;
A21 represents Asp, or Ala;
A23 represents Val, or Ile;
A24 represents Asn, Gln, or Asp;
A25 represents Trp, or Thr;
A27 represents Leu, Glu, Ser, Lys, or Val;
A28 represents Ala, Ser or Arg;
A29 represents Gln, Aib, Gly, Ala, Thr, Ser or Lys;
A30 represents Lys Gly or ψ;
A31 represents Gly, Pro Gly-OH, or a deletion;
A32 represents Lys, Ser, or a deletion;
A33 represents Lys, Ser, or a deletion;
A34 represents Asn, Gly, or a deletion;
A35 represents Asp, Ala, or a deletion;
A36 represents Trp, Pro, or a deletion;
A37 represents Lys, Pro, or a deletion;
A38 represents His, Pro, or a deletion;
A39 represents Asn, Ser, or a deletion;
A40 represents Ile, or a deletion;
A41 represents Thr, or a deletion;
A42 represents Gln, or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

16. A method for treating emesis in a subject, the method comprising administering a therapeutically effective amount of a GIP receptor agonist peptide, or a salt thereof, having an amino acid sequence of formula (VI): P¹-Tyr-A2-Glu-Gly-Thr-Phr-A7-Ser-Asp-Thr-Ser-Ile-A13-A14-Asp-Lys-Ile-A18-Gln-A20-A21-A22-Val-A24-Trp-Leu-A27-Ala-A29-A30-A31-A32-A33-A34-A35-A36-A37-A38-A39-A40-A41-A42-P²,
wherein
P¹ represents a group represented by formula
R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib or Ala;
A7 represents Ile, or s, Thr;
A13 represents Aib, or Ala;
A14 represents Leu, or Met;
A18 represents Ala, or His;
A20 represents Gln, Arg, or ψ;
A21 represents Asp, or Ala;
A22 represents Phe, Naphthyl-Ala, or αMePhe;
A24 represents Asn, or Gln;
A27 represents Leu or Ile;
A29 represents Gln, Aib, or Gly;
A30 represents Lys or Gly;
A31 represents Gly or Pro;
A32 represents Lys, or Ser;
A33 represents Lys, or Ser;
A34 represents Asn, Gly, or Gln;
A35 represents Asp, Glu, or Ala;
A36 represents Trp, or Pro;
A37 represents Lys, or Pro;
A38 represents His, or Pro;
A39 represents Asn, Gln, or Ser;
A40 represents Ile, or a deletion;
A41 represents Ile, Thr, or a deletion;
A42 represents Gln, or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

17. A method for treating emesis in a subject, the method comprising administering a therapeutically effective amount of a GIP receptor agonist peptide, or a salt thereof, having an amino acid sequence of formula (VII): P¹-Tyr-A2-Glu-Gly-Thr-Phr-A7-Ser-Asp-Thr-Ser-A12-A13-A14-Asp-A16-A17-A18-Gln-A20-A21-Phe-Val-A24-Trp-A26-A27-Ala-A29-A30-A31-A32-A33-A34-A35-A36-A37-A38-A39-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2 represents Aib or Ala;
A7 represents Ile, Leu, or Thr;
A12 represents Ile or Leu;
A 13 represents Aib, or Ala;
A14 represents Leu, Met or ψ;
A16 represents Lys or Arg;
A17 represents Ile or Aib;
A18 represents Ala, His, or ψ;
A20 represents Gln, or Aib;
A21 represents Asp, Ala, Asn or ψ;
A24 represents Asn, Glu, or Gln;
A26 represents Leu or Ile;
A27 represents Leu or Ile;
A29 represents Gln or ψ;
A30 represents Lys, Arg, ψ, Ser or Gln;
A31 represents Gly, Pro, or a deletion;
A32 represents Ser, or a deletion;
A33 represents Ser, or a deletion;
A34 represents Gly, or a deletion;
A35 represents Ala, or a deletion;
A36 represents Pro, or a deletion;
A37 represents Pro, or a deletion;
A38 represents Pro, or a deletion;
A39 represents Ser, or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.
GIP receptor agonistGIP receptor agonistGIP receptor agonistGIP receptor agonist.

18. A method for treating emesis in a non-type 2 diabetes mellitus subject, the method comprising administering a therapeutically effective amount of a GIP receptor agonist peptide, or a salt thereof, said GIP receptor agonist peptide having an amino acid sequence of formula (I): P′-A1-A2-A3-A4-A5-A6-A7-A8-A9-A10-A11-A12-A13-A14-A15-A16-A17-A 18-A19-A20-A21-A22-A23-A24-A25-A26-A27-A28-A29-A30-A31-A32-A33-A34-A35-A36-A37-A38-A39-A40-A41-A42-A43-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, Phe, D-Tyr, mono-halo-Phe, bis-halo-Phe, mono-halo-Tyr, bis-halo-Tyr, mono-halo-D-Phe, bis-halo-D-Phe, mono-halo-D-Tyr, bis-halo-D-Tyr, des-amino-Phe, or des-amino-Tyr;
A2 represents Ala, Aib, D-Ala, Gly, Ser, or Sar;
A3 represents Glu, or Pro;
A4 represents Gly, or Ser;
A5 represents Thr, D-Iva, Glu, Iva, or Ser;
A6 represents Phe, Iva, Val, Ala, Aib, Cha, or α-methyl-Leu;
A7 represents Ile, Lys, Ala, Aib, Thr, Cha, D-Leu, Ile, Arg, or Val;
A8 represents Ser, Ala, ψ, or Aib;
A9 represents Asp, Leu, ψ, Phe, Glu, or Gln;
A10 represents Tyr, Leu, Ser, Cha, or ψ;
A11 represents Ser, Aib, A5c, A6c, D-Iva, or Iva;
A12 represents Ile, Lys, Glu, Asp, Ala, Aib, Lys-Ac, Ser, α-methyl-Phe, or ψ;
A 13 represents Ala, Aib, Tyr, D-Iva, ψ, Gln, Leu, Glu, or Iva;
A14 represents Met, Nle, Leu, α-methyl-Leu, or ψ;
A15 represents Asp, Glu, Lys, Ser, Tyr, ψ, or Asn;
A16 represents Lys, Ala, Ser, Glu, Arg, Aib, Lys-Ac, or ψ;
A17 represents Ile, Lys, Arg, Aib, Gln, Glu, Lys-Ac, or ψ;
A18 represents His, Arg, Ala, Aib, D-Iva, Phe, Iva, Leu, Ser, Trp, or ψ;
A19 represents Gln, Lys, Glu, Ala, Val, Ser, Aib, Arg, or ψ;
A20 represents Gln, Lys, Ala, His, Arg, Aib, Asp, Gly, or ψ;
A21 represents Asp, Leu, Asn, Asp, Glu, Ala, Leu, Ser, Aib, or ψ;
A22 represents Phe, α-methyl-Phe, Naphthyl-Ala, Asn, Ala, Trp, or ψ;
A23 represents Val, Ile, or ψ;
A24 represents Asn, Asp, Glu, Ala, Aib, Gln, Glu, Lys, Lys-Ac, Leu, Nle, Arg, Ser, or ψ;
A25 represents Trp, Tyr, Glu, Phe, Arg, α-methyl-Phe, or ψ;
A26 represents Leu, Aib, Iva, Leu, Nle, or ψ;
A27 represents Leu, Glu, Ser, Lys, Val, Ile, Nle, or ψ;
A28 represents Ala, Ser, Arg, Leu, Met, Lys, Lys-Ac, Aib, or ψ;
A29 represents Gln, Gly, Ala, Thr, Ser, Lys, Aib, ψ, or a deletion;
A30 represents Lys, Arg, Pro, Gly, Glu, Lys-Ac, ψ, or a deletion;
A31 represents Phe, Pro, Gly, ψ, or a deletion;
A32 represents Lys, Ser, Gly, ψ, or a deletion;
A33 represents Lys, Ser, Gly, Ile, Ser, ψ, or a deletion;
A34 represents Asn, Gly, Ala, Gln, ψ, or a deletion;
A35 represents Asp, Ala, Pro, Ser, Glu, ψ, or a deletion;
A36 represents Trp, Pro, Gly, ψ, or a deletion;
A37 represents Lys, Pro, Gly, ψ, or a deletion;
A38 represents His, Pro, Ser, Gly, ψ, or a deletion;
A39 represents Asn, Ser, Gly, Asn, Lys, Gln, ψ, or a deletion;
A40 represents Ile, Arg, Glu, Lys, Ser, Lys-Ac, Arg, ψ, or a deletion;
A41 represents Ile, Thr, Gly, ψ, or a deletion;
A42 represents Gln, Gly, ψ, or a deletion;
A43 represents ψ, or a deletion;
wherein ψ is a residue independently selected from Lys, Arg, Orn, and Cys and wherein the side chain of said residue is substituted, with the proviso that the GIP receptor agonist peptide is not native human GIP having an amino acid sequence as set forth in SEQ ID NO: 1.

19. A method for treating emesis in a non-type 2 diabetes mellitus subject, the method comprising administering a therapeutically effective amount of a GIP receptor agonist peptide, or a salt thereof, said GIP receptor agonist peptide having an amino acid sequence of formula (IV): P¹-A1-A2-A3-Gly-A5-A6-A7-A8-A9-A10-A11-A12-A13-A14-A15-A16-A17-A18-A19-A20-A21-A22-A23-A24-A25-A26-A27-A28-A29-A30-A31-A32-A33-A34-A35-A36-A37-A38-A39-A40-A41-A42-A43-P²,
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A1 represents Tyr, D-Tyr, Phe, or 3,5 di-Br-Tyr;
A2 represents Aib, Ala, or D-Ala;
A3 represents Glu, or Pro;
A5 represents Thr, or Glu;
A6 represents Iva, Phe, or Val;
A7 represents Ile, Lys, Thr, or Val;
A8 represents Ser, or Lys(R);
A9 represents Asp, Leu, Lys(R), or Phe;
A10 represents Tyr, or Lys(R);
A11 represents Aib, A5c, A6c, or Ser;
A12 represents Ile, Glu, or Lys(R);
A13 represents Aib, Ala, Gln, Glu, Leu, Lys(R), Tyr, or D-Iva;
A14 represents Leu, Met, Lys(R), or Nle;
A15 represents Asp, Glu, Lys, Lys(R), Ser, or Tyr;
A16 represents Arg, Ala, Lys(R), or Lys;
A17 represents Aib, Glu, Lys(R), Gln, or Ile;
A18 represents Ala, Lys(R), or His;
A19 represents Gln, Lys(R), Glu, Val, Ala, or Ser;
A20 represents Aib, Lys(R), Arg, Ala, or Gln;
A21 represents Asn, Asp, Ala, Glu, Lys(R), Leu, Aib, or Ser;
A22 represents Phe, Lys(R), Naphthyl-Ala, or αMePhe;
A23 represents Ile, Lys(R), or Val;
A24 represents Arg, Asn, Ala, Gln, Glu, Asp, Lys, or Lys(R);
A25 represents Trp, Lys(R), or αMePhe;
A26 represents Aib, Lys(R), Iva, or Leu;
A27 represents Leu, Lys(R), Val, or Ile;
A28 represents Ala, Arg, Lys, or Lys(R);
A29 represents Gln, Aib, or Gly;
A30 represents Arg, Lys, Pro, Gly, Lys(R), or a deletion;
A31 represents Gly, Pro, or a deletion;
A32 represents Lys, Ser, or a deletion;
A33 represents Lys, Ser, or a deletion;
A34 represents Asn, Gly, Ala, Gln, or a deletion;
A35 represents Asp, Glu, Pro, Ala, or a deletion;
A36 represents Trp, Pro, or a deletion;
A37 represents Lys, Pro, Lys(R), or a deletion;
A38 represents His, Pro, Ser, or a deletion;
A39 represents Asn, Gln, Lys, Ser, or a deletion;
A40 represents Arg, Glu, Ile, Lys, Lys(R), or a deletion;
A41 represents Ile, Thr, or a deletion;
A42 represents Gln, or a deletion;
with the proviso that the GIP receptor agonist peptide is not a peptide having an amino acid sequence of SEQ ID NOs: 4 to 569 disclosed in PCT Application No. PCT/JP2018/013540, and wherein any one or two amino acids selected from A8 to A42 optionally represent Lys(R), and the (R) in Lys(R) represents a substituent group, or a salt thereof.

20. The method according to any one of claims 1 to 19, wherein the (R) group in Lys(R) represents X-L-, L represents a bivalent linker comprising PEG and/or two or more amino acids, and X represents a substituent group, or a salt thereof.

21. The method according to any one of claims 1 to 19, wherein the (R) group in Lys(R) represents X-L-, L represents a bond or a bivalent substituent group, and X represents an optionally substituted hydrocarbon group, or a salt thereof.

22. The method according to any one of claims 1 to 19, wherein the (R) group in Lys(R) represents X-L-, L represents a bond or a bivalent substituent group, and X represents a C₆-C₂₀ monoacid, a C₆-C₂₀ diacid or an acetyl group, or a salt thereof.

23. The method according to any one of claims 1 to 19, wherein the GIP receptor agonist peptide is represented by formula: Ac-YAEGTFISDYSIAMDKIHQQDFVNWLLAQKGKKNDWKHNIIQ-OH (SEQ ID NO: 4), or a salt thereof.

24. The method according to any one of claims 1 to 19, wherein the GIP receptor agonist peptide is represented by formula: Y(D-Ala)EGTFISDYSIAMDKIHQQDFVNWLLAQKGKKNDW(Lys(R))HNIIQ-NH₂ (SEQ ID NO: 5), or a salt thereof.

25. The method according to any one of claims 1 to 19, wherein the GIP receptor agonist peptide is represented by formula: YAEGTFISDYSIAMD(Lys-(R)IHQQDFVNWLLAQKGKKNDWKHNIIQ-NH₂ (SEQ ID NO: 6), or a salt thereof.

26. The method according to any one of claims 1 to 19, wherein the GIP receptor agonist peptide is represented by formula: YD-AlaEGTFISDYSIAMDKIHQQDFVNWLLAQK-OH (SEQ ID NO: 7), or a salt thereof.

27. The method according to any one of claims 1 to 19, wherein the GIP receptor agonist peptide is represented by formula: YAibEGTFISDYSIAMDKK(g-Glu-C₁₆)HQQDFVNWLLAQKGKKNDWKHNIIQ-OH (SEQ ID NO: 8), or a salt thereof.

28. The method according to any one of claims 1 to 19, wherein the GIP receptor agonist peptide is represented by formula: FAibEGTFISDYX*IA(Nle)DKIHQQDFVNWLLAQKGKKNDWKHNITQ-OH (X* is 1-amino,1-cyclopentane carboxylic acid; SEQ ID NO: 12), wherein R represents a substituent group, or a salt thereof.

29. The method according to any one of claims 1 to 19, wherein the GIP receptor agonist peptide is represented by formula: (3,5-diBr-Tyr)AibEGTFISDYSIAib(Nle)DKIHQQDFVNWLLAQKGKKNDWKHNITQ-OH (SEQ ID NO: 11), wherein R represents a substituent group, or a salt thereof.

30. The method according to any one of claims 1 to 19, wherein the GIP receptor agonist peptide is represented by formula:
(SEQ ID NO: 15)
YAPGTFISDYSIAMDKIHQQDFVNWLLAQIKPSSGAPPPS-NH₂,
wherein R represents a substituent group, or a salt thereof.

31. The method according to any one of claims 1 to 19, wherein the GIP receptor agonist peptide is represented by formula: Y(D-Ala)EGTFISDYSIAMDKIHQQDFVNWLLAQKPSSGAPPPS-NH₂ (SEQ ID NO: 14), wherein R represents a substituent group, or a salt thereof.

32. The method according to any one of claims 1 to 19, wherein the GIP receptor agonist peptide is represented by formula: YAibEGTFTSDYSIYMEKEAV(Lys(R))EFIAWLVKG-OH (SEQ ID NO: 9), wherein (R) in (Lys(R)) represents a substituent, or a salt thereof.

33. The method according to any one of claims 1 to 19, wherein the GIP receptor agonist peptide is represented by formula:
(SEQ ID NO: 10)
Ac-(D-Tyr)AEGTFISDYSIAMDAIHQQDFVNWLLAQ(LysR)-NH₂,
wherein (R) in (Lys(R)) represents a substituent, or a salt thereof.

34. The method according to any one of claims 1 to 19, wherein the GIP receptor agonist peptide is represented by formula: YAibEGTFTSDYSIYLDKQAAAibEFVNWLLAGGPSSGAPPPS(Lys(R))—NH₂ (SEQ ID NO: 13), wherein (R) in (Lys(R)) represents a substituent, or a salt thereof.

35. The method according to any one of claims 1 to 19, wherein the GIP receptor agonist peptide is represented by formula: YD-AlaEGTFISDYSIAMD(Lys(R))IHQQDFVNWLLAQKGKKNDWKHNIIQ-NH₂ (SEQ ID NO: 16), wherein (R) in (Lys(R)) represents a substituent, or a salt thereof.

36. The method according to any one of claims 1 to 19, wherein the GIP receptor agonist peptide is represented by formula:
(SEQ ID NO: 17)
YD-AlaEGTFISDYSIAMDKIHQQDFVNWLLAQKGKKNDWKHNIIQ-NH₂.

37. The method according to any one of claims 1 to 19, wherein the GIP receptor agonist peptide is represented by formula: YAibEGTFTSDYSIYMEKEAVREFIAWLVKGGPSSGAPPPS(Lys(R))—NH₂ (SEQ ID NO: 18), wherein (R) in (Lys(R)) represents a substituent, or a salt thereof.

38. The method according to any one of claims 1 to 19, wherein the GIP receptor agonist peptide is represented by formula: YAibEGTFTSDYSIAibLDKIAQRAFVQWLIAAibKGKKQEWKHQITQ(Lys(R))—NH₂ (SEQ ID NO: 19), wherein (R) in (Lys(R)) represents a substituent, or a salt thereof.

39. The method according to any one of claims 1 to 19, wherein the GIP receptor agonist peptide is represented by formula: YAibEGTFTSDYSIYLDKQAAAibEFVNWLLAGGPSSGAPPPS-(Lys(R))—NH₂ (SEQ ID NO: 20), wherein (R) in (Lys(R)) represents a substituent, or a salt thereof.

40. The method according to any one of claims 1 to 19, wherein the GIP receptor agonist peptide is represented by formula: YAibEGTFTSDYSIAibLDKIAQ(Lys(R))AFVQWLIAGGPSSGAPPPS-NH₂ (SEQ ID NO: 21), wherein (R) in (Lys(R)) represents a substituent, or a salt thereof.

41. The method according to any one of claims 1 to 19, wherein the GIP receptor agonist peptide is represented by formula: YAibEGTFISDYSIAMDKIHQQDFVNWLLAQKGKKNDWKHNITQ(Lys(R))—OH (SEQ ID NO: 22), wherein (R) in (Lys(R)) represents a substituent, or a salt thereof.

42. The method according to any one of claims 1 to 19, wherein the GIP receptor agonist peptide is represented by formula: YAibEGTFISDYSIELDK(Lys(R))AAQAFIEWLLAQGPSSGAPPPS-NH₂ (SEQ ID NO: 23), wherein (R) in (Lys(R)) represents a substituent, or a salt thereof.

43. The method according to any one of claims 1 to 19, wherein the GIP receptor agonist peptide is represented by formula: YAibEGTFISDYSIELDKIAAQDFIEWLLAGPSSGAPPPS(Lys(R))—NH₂ (SEQ ID NO: 24), wherein (R) in (Lys(R)) represents a substituent, or a salt thereof.

44. The method according to any one of claims 1 to 19, wherein the GIP receptor agonist peptide is represented by formula: YAibEGTFISDYS(Lys(R))ELDKIAQRAFIEWLLAQGPSSGAPPPS-NH₂(SEQ ID NO: 25), wherein (R) in (Lys(R)) represents a substituent, or a salt thereof.

45. The method according to any one of claims 1 to 19, wherein the GIP receptor agonist peptide is represented by formula: YAibEGTFISDYSIELEK(Lys(R))AQRAFVEWLLAQGPSSGAPPPS-NH₂ (SEQ ID NO: 26), wherein (R) in (Lys(R)) represents a substituent, or a salt thereof.

46. The method according to any one of claims 1 to 19, wherein the GIP receptor agonist peptide is represented by formula: YAibEGTFISDYSIALDKIHQQ(Lys(R))FVNWLLAQRG-OH (SEQ ID NO: 27), wherein (R) in (Lys(R)) represents a substituent, or a salt thereof.

47. The method according to any one of claims 1 to 19, wherein the GIP receptor agonist peptide is represented by formula: YAibEGTFISDYSIALDKI(Lys(R))QQDFVNWLLAQRG-OH (SEQ ID NO: 28), wherein (R) in (Lys(R)) represents a substituent, or a salt thereof.

48. The method according to any one of claims 1 to 19, wherein the GIP receptor agonist peptide is represented by formula:
(SEQ ID NO: 29)
YAibEGTFISDYSIALDK(Lys(R))HQQDFVNWLLAQRG-OH,
wherein (R) in (Lys(R)) represents a substituent, or a salt thereof.

49. The method according to any one of claims 1 to 19, wherein the GIP receptor agonist peptide is represented by formula: YAibEGTFISDYSIALDK(Lys(R))HQQDFVNWLLAQR-NH₂ (SEQ ID NO: 30), wherein (R) in (Lys(R)) represents a substituent, or a salt thereof.

50. The method according to any one of claims 1 to 19, wherein the GIP receptor agonist peptide is represented by formula: YAibEGTFISDYSIALDKIHQQ(Lys(R))FVNWLLAQR-NH₂ (SEQ ID NO: 31), wherein (R) in (Lys(R)) represents a substituent, or a salt thereof.

51. The method according to any one of claims 1 to 19, wherein the GIP receptor agonist peptide is represented by formula: YAibEGTFISDYSIALDKIHQQ(Lys(R))FVNWLLAQRPSSGAPPPS-NH₂ (SEQ ID NO: 32), wherein (R) in (Lys(R)) represents a substituent, or a salt thereof.

52. The method according to any one of claims 1 to 19, wherein the GIP receptor agonist peptide is represented by formula: YAibEGTFISDYSIALDKI(Lys(R))QQDFVNWLLAQRPSSGAPPPS-NH₂ (SEQ ID NO: 33), wherein (R) in (Lys(R)) represents a substituent, or a salt thereof.

53. The method according to any one of claims 1 to 19, wherein the GIP receptor agonist peptide is represented by formula: YAibEGTFISDYSIALDKIHQQDFVNWLLAQ(Lys(R))G-OH (SEQ ID NO: 34), wherein (R) in (Lys(R)) represents a substituent, or a salt thereof.

54. The method according to any one of claims 1 to 19, wherein the GIP receptor agonist peptide is represented by formula:
(SEQ ID NO: 35)
YAibEGTFISDYS(Lys(R))ALDKIHQQDFVNWLLAQRG-OH,
wherein (R) in (Lys(R)) represents a substituent, or a salt thereof.

55. The method according to any one of claims 1 to 19, wherein the GIP receptor agonist peptide is represented by formula: YAibEGTFTSDYSIAibLDKIAQ(Lys(R))AFVQWLIAGGPSSGAPPPS-NH₂ (SEQ ID NO: 36), wherein (R) in (Lys(R)) represents a substituent, or a salt thereof.

56. The method according to any one of claims 1 to 19, wherein the GIP receptor agonist peptide is represented by formula: YAibEGTFTSDYSIAibLDKIAQ(Lys(R))AFVQWLIAGGPSSGAPPPS-NH₂ (SEQ ID NO: 37), wherein (R) in (Lys(R)) represents a substituent, or a salt thereof.

57. The method according to any one of claims 1 to 19, wherein the GIP receptor agonist peptide, or a salt thereof, has at least 90%, or at least 95%, or at least 99%, or at least 100% sequence identity to a peptide represented by formulas (I), (II), (III), (IV), (V), (VI), or (VII).

58. The method according to any one of claims 1 to 19, wherein the GIP receptor agonist peptide, or a salt thereof, has at least 95%, or at least 96%, or at least 97%, or at least 99%, or at least 100% sequence identity to a peptide represented by formulas (I), (II), (III), (IV), (V), (VI), or (VII).

59. The method according to any one of claims 1 to 19, wherein the GIP receptor agonist peptide, or a salt thereof, has at least 100% sequence identity to a peptide represented by formulas (I), (II), (III), (IV), (V), (VI), or (VII).

60. The use of a GIP receptor agonist peptide of any one of claims 1 to 59, or a salt thereof, for the manufacture of a medicament for the treatment of emesis.

61. A pharmaceutical composition for use in the treatment of emesis, the pharmaceutical composition comprising a GIP receptor agonist peptide of any one of claims 1 to 59, or a salt thereof.

62. The method according to any one of claims 1 to 59, wherein the GIP receptor agonist peptide or a salt thereof, is formulated into medicament.

63. The method according to claim 62, wherein the GIP receptor agonist peptide has a selectivity ratio of GLPr/GIPr of greater than 10, or greater than 100, or greater than 1,000.

64. The method according to any one of claims 1 to 59, and 62-63, wherein the GIP receptor agonist peptide or medicament or pharmaceutical composition is administered to treat emesis as a monotherapy.

65. The method according to any one of claims 1 to 59, and 62-63, wherein the emesis includes nausea and/or vomiting.

66. The method according to claim 65, wherein the nausea and/or vomiting is a result of cyclic vomiting syndrome or chemotherapy.

67. The method of any one of claims 1 to 59, and 62-63, wherein the subject is a non-type 2 diabetes mellitus subject.

68. The method according to any one of claims 1 to 59, and 62-63, wherein the emesis is delayed emesis or anticipatory emesis.

69. The method of claim 1, the use of claim 60, the medicament of claim 62, or the method of claim 65, where the vomiting or the nausea is caused by one or more conditions or causes selected from the following (1) to (10):
(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome, chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium;
(6) Vomiting and nausea caused by a vestibular disorder such as motion sickness or dizziness;
(7) Opioid withdrawal;
(8) Pregnancy including hyperemesis gravidarium;
(9) A vestibular disorder such as motion sickness or dizziness; and
(10) A physical injury causing local, systemic, acute or chronic pain.

70. The method according to claim 69, wherein the emesis is caused by cyclic vomiting syndrome or administration of a chemotherapeutic agent or chemotherapy.

71. The method according to any one of claims 1 to 19, wherein the GIP receptor agonist peptide further comprises a modification by fatty acid addition at an epsilon amino group of at least one lysine residue.

72. The method according to claim 71, wherein the modification is the linking of a C-16 palmitate group to the epsilon amino group of a lysine residue.

73. The method according to claim 72, wherein the lysine residue is selected from the group consisting of: Lys₁₆, Lys₂₀, Lys₃₇, and Lys₄₀.

74. The method according to any one of claims 1 to 59, wherein the GIP receptor agonist peptide is formulated as a medicament comprising a GIP receptor agonist peptide, or a salt thereof and at least one pharmaceutically acceptable excipient.

75. The method according to claim 74, wherein the medicament selectively activates the GIP receptor over the GLP receptor.

76. The method according to claim 75, wherein the medicament has a selectivity ratio of (GLPr EC₅₀/GIPr EC₅₀) of greater than 10.

77. The method according to claim 76, wherein the medicament has a selectivity ratio of (GLPr EC₅₀/GIPr EC₅₀) of greater than 100.

78. The method according to claim 77, wherein the medicament has a selectivity ratio of (GLPr EC₅₀/GIPr EC₅₀) of greater than 1000.

79. The method according to any one of claims 1 to 59, and 62-78, wherein the emesis is delayed emesis.

80. The method according to any one of claims 1 to 59, and 62-79, wherein the emesis is anticipatory emesis.

81. The method according to any one of claims 1 to 59, and 62-80, wherein emesis is treated in the subject without inducing anxiety or sedation in the subject.

82. The method according to any one of claims 1 to 59, and 62-81, wherein emesis is treated in the subject without inducing suppression of glucagon secretion when plasma glucose levels are above fasting levels.

83. The method according to any one of claims 1 to 59, and 62-82, wherein emesis is treated in the subject without substantially activating the GLP-1 receptor.

84. The method according to any one of claims 1 to 62, wherein emesis is treated in the subject without concomitant, subsequent, or prior administration of a GLP-1 receptor agonist.

85. The method according to any one of claims 1 to 59, and 62-82, wherein emesis is treated in a subject not taking a medicament to control a metabolic syndrome disorder.

86. The method according to any one of claims 1 to 59, and 62-82, wherein emesis is treated in a subject taking a medicament to control a metabolic syndrome disorder.

87. The method according to any one of claim 85 or 86, wherein the metabolic syndrome disorder is type 2 diabetes mellitus or obesity.

88. The method according to any one of claims 1 to 59, and 62-87, wherein the GIP receptor agonist peptide is a suppressant for vomiting or nausea.

89. The method according to any one of claims 1 to 59, and 62-88, wherein the emesis is caused by or causes cyclic vomiting syndrome, or nausea or vomiting associated with administration of a chemotherapeutic agent.

90. The method according to claim 89, wherein the chemotherapeutic agent comprises: (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome.

91. The method according to any one of claims 1 to 17, wherein the subject has type 2 diabetes mellitus.

92. The method according to any one of claims 1 to 59, and 62-91, wherein the GIP receptor agonist peptide or medicament is administered subcutaneously, intravenously, intramuscularly, intraperitonealy, orally or via inhalation.

93. The method according to any one of claims 1 to 59, and 62-92, wherein the effective amount of the GIP receptor agonist peptide administered to the subject is about 0.01 to 0.5 mg/kg/day, 0.1 to 5 mg/kg/day, 5 to 10 mg/kg/day, 10 to 20 mg/kg/day, 20 to 50 mg/kg/day, 10 to 100 mg/kg/day, 10 to 120 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.

94. The method according to any one of claims 1 to 59, and 62-93, wherein the subject is human.

95. The method according to any one of claims 1 to 59, and 62-94, wherein the GIP receptor agonist peptide or medicament is administered to the subject before, during, or after the subject develops the disease-state.

96. The method according to any one of claims 1 to 59, and 62-95, wherein the GIP receptor agonist peptide or medicament is administered to the subject 1-3 times per day or 1-7 times per week.

97. The method according to any one of claims 1 to 59, and 62-96, wherein the GIP receptor agonist peptide or medicament is administered to the subject for 1-5 days, 1-5 weeks, 1-5 months, or 1-5 years.
